SEQUENCE LISTING (5 sequences)

SEQ ID NO: 1 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 2 - protein, 233 aa, homo sapiens
MGVPTQLLLLGLTVVVVRCDIQMTQSPASLSASVGETVTITCGASENIYGGLTWYQRRRGKSPQLLIYGASNLADGMSSRFTGSGSGRQFSLKISGVHPDDVATYYCQNVFTTPYTFGGGTRLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 472 aa, Homo sapiens
MRVLILLCLFTVFPGILSDVRLQESGPDLVKPSQSLSLTCTVTGYSITRAYNWHWIRQFPGNKLEWMGYILYSGTTDYNPSLKSRISFTRDTSKNQLILQLDSVTTDDTGTYYCARAGYDGGMDYWGQGVSVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 4 - protein, 234 aa, Homo sapiens
MKSQTQVFVFLLLCVSGVHGSIVMTQTPKFLLVSPGDRVTITCKASQSVGHDVSWYRQKPGQSPKVLIYYASNRYTGVPDRFTGSGYGTDFTFTVRSVRTEDLAVYFCQQDYTSPLTFGTGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 5 - protein, 473 aa, Homo sapiens
MGWNYIILFLVTTATDVHSQAQLQQPGAELVKPGASVNLSCKASGYTFTNYWIHWVSQRPGQGLEWIGEINPSDGRTYYNEKFKNKVILTVDEASTTAYLQLSSLTSEDSAVYYCTSRYYIYGVDYWGQGTTLTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQIGWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

(19) United States
(12) Patent Application Publication
(10) Pub. No.: US 2019/0137523 A1
(43) Pub. Date: May 9, 2019

(54) METHOD AND MEANS FOR DETECTING THE LEVEL OF TOTAL VEGF-A

(71) Applicant: Hoffmann-La Roche Inc., Little Falls, NJ (US)

(72) Inventors: Alfred Engel, Weilheim (DE); Johann Karl, Peissenberg (DE); Christina Rabe, Bad Heilbrunn (DE); Michael Schraeml, Penzberg (DE); Monika Soukupova, Wessobrunn (DE); Peter Stegmueller, Augsburg (DE); Norbert Wild, Wolfratshausen (DE)

(73) Assignee: Hoffmann-La Roche Inc., Little Falls, NJ (US)

(21) Appl. No.: 16/245,016

(22) Filed: Jan. 10, 2019

Related U.S. Application Data
(63) Continuation of application No. PCT/EP2017/067707, filed on Jul. 13, 2017.

(30) Foreign Application Priority Data
Jul. 15, 2016 (EP) 16179780.8

(51) Int. Cl. G01N 33/74; C07K 16/22

(52) U.S. Cl. CPC G01N 33/74; C07K 16/22; C07K 2317/40; C07K 2317/565; G01N 2333/49

(57) ABSTRACT

The present invention relates to a method for measuring the level of VEGF-A in the presence of a VEGF-A antagonist, kits comprising means for detecting VEGF-A in the presence of a VEGF-A antagonist, compositions of matter comprising a first and a second antibody suitable for detecting the level of VEGF-A in the presence of a VEGF-A antagonist, as well as methods of detecting a complex comprising human VEGF-A and a non-human or chimeric protein.

### CROSS REFERENCE TO RELATED APPLICATIONS

This application is a continuation of International Application No. PCT/EP2017/067707, filed Jul. 13, 2017, which claims the benefit of EP Application No. 16179780.8, filed Jul. 15, 2016, each of which are incorporated herein by reference in its entirety.

### SEQUENCE LISTING

This application contains a Sequence Listing which has been submitted electronically in ASCII format and is hereby incorporated by reference in its entirety. Said ASCII copy, created on Jan. 3, 2019, is named P33749-US_sequence listing and is 14,640 bytes in size.

### FIELD OF THE INVENTION

The present invention relates to a method for measuring the level of VEGF-A in the presence of a VEGF-A antagonist, kits comprising means for detecting VEGF-A in the presence of a VEGF-A antagonist, compositions of matter comprising a first and a second antibody suitable for detecting the level of VEGF-A in the presence of a VEGF-A antagonist, as well as methods of detecting a complex comprising human VEGF-A and a non-human or chimeric protein.

### BACKGROUND OF THE INVENTION

Cancer is one of the most deadly threats to human health. In the U.S. alone, cancer affects nearly 1.3 million new patients each year, and is the second leading cause of death after cardiovascular disease, accounting for approximately 1 in 4 deaths. Solid tumors are responsible for most of those deaths. Although there have been significant advances in the medical treatment of certain cancers, the overall 5-year survival rate for all cancers has improved only by about 10% in the past 20 years. Cancers, or malignant tumors, metastasize and grow rapidly in an uncontrolled manner, making timely detection and treatment extremely difficult. Depending on the cancer type, patients typically have several treatment options available to them including chemotherapy, radiation and antibody-based drugs. Diagnostic methods useful for predicting clinical outcome from the different treatment regimens would greatly benefit clinical management of these patients.

It is now well established that angiogenesis is implicated in the pathogenesis of a variety of disorders. These include solid tumors, intra-ocular neovascular syndromes such as proliferative retinopathies or age-related macular degeneration (AMD), rheumatoid arthritis, 20 and psoriasis (Folkman et al. J. Biol. Chem. 267:10931-10934 (1992); Klagsbrun et al. Annu. Rev. Physiol. 53:217-239 (1991); and Gamer A, Vascular diseases. In: Pathobiology of ocular disease. A dynamic approach. Gamer A, Klintworth G K, Eds. 2nd Edition (Marcel Dekker, N.Y., 1994), pp 1625-1710). In the case of solid tumors, the neovascularization allows the tumor cells to acquire a growth advantage and proliferative autonomy compared to the normal cells. Accordingly, a correlation has been observed between density of microvessels in tumor sections and patient survival in breast cancer as well as in several other tumors (Weidner et al. N Engl J Med 324:1-6 (1991); Horak et al. Lancet 340:1120-1124 (1992); and Macchiarini et al. Lancet 340: 145-146 (1992)).

A key role has been established for vascular endothelial growth factor (VEGF) in the regulation of normal and abnormal angiogenesis (Ferrara et al. Endocr. Rev. 18:4-25 (1997)). The finding that the loss of even a single VEGF allele results in embryonic lethality points to an irreplaceable role played by this factor in the development and differentiation of the vascular system (Ferrara et al., supra). VEGF has been also shown to be a key mediator of neovascularization associated with tumors and intra-ocular disorders (Ferrara et al., supra). The VEGF mRNA is overexpressed by the majority of human tumors examined (Berkman et al. J Clin Invest 91:153-159 (1993); Brown et al. Human Pathol. 26:86-91 (1995); Brown et al. Cancer Res. 53:4727-4735 (1993); Mattern et al. Brit. J. Cancer. 73:931-934 (1996); and Dvorak et al. Am J. Pathol. 146:1029-1039 (1995)). Also, the concentration of VEGF in eye fluids is highly correlated to the presence of active proliferation of blood vessels in patients with diabetic and other ischemia-related retinopathies (Aiello et al. N. Engl. J. Med. 331: 1480-1487 (1994)). Furthermore, studies have demonstrated the localization of VEGF in choroidal neovascular membranes in patients affected by acute macular degeneration (AMD) (Lopez et al. Invest. Ophtalmo. Vis. Sci. 37:855-868 (1996)).

The ability to measure endogenous VEGF levels depends on the availability of sensitive and specific assays. Colorimetric, chemiluminescence, and fluorometric based enzyme-linked immunosorbent assays (ELISAs) for VEGF have been reported. Houck et al., supra, (1992); Yeo et al. Clin. WO 2008/060777 PCT/US2007/080310 Chem. 38:71 (1992); Kondo et al. Biochim. Biophys. Acta 1221:211 (1994); Baker et al. Obstet. Gvnecol. 86:815 (1995); Hanatani et al. Biosci. Biotechnol. Biochem. 59:1958 (1995); Leith and Michelson Cell Prolif. 28:415 (1995); Shifren et al. J. Clin. Endocrinol. Metab. 81:3112 (1996); Takano et al. Cancer Res. 56:2185 (1996); Toi et al. Cancer 77:1101 (1996); Brekken et al. Cancer Res. 58:1952 (1998); Obermair et al. Br. J. Cancer 77:1870-1874 (1998); Webb et al. Clin. Sci. 94:395-404 (1998). For example, Houck et al., supra (1992) describe a colorimetric ELISA that appears to have ng/ml sensitivity, which may not be sensitive enough to detect endogenous VEGF levels. Yeo et al., supra (1992) describe a two-site time-resolved immunofluorometric assay, however, no VEGF was detected in normal sera (Yeo et al. Cancer Res. 53:2912 (1993)). Baker et al., supra (1995), using a modified version of this immunofluorometric assay, reported detectable levels of VEGF in plasma from pregnant women, with higher levels observed in women with preeclampsia. Similar data in pregnant women were reported by Anthony et al. Ann. Clin. Biochem. 34:276 (1997) using a radioimmunoassay. Hanatani et al., supra (1995) developed a chemiluminescent ELISA capable of measuring circulating VEGF and report VEGF levels in sera from 30 normal individuals (male and female) from 8-36 pg/ml. Brekken et al, supra (1998) described ELISA assays using antibodies having binding preference to either the VEGF alone or the VEGF:Flk-1 complex. An ELISA kit for VEGF detection is commercially available from R&D Systems (Minneapolis, Minn.). The R&D VEGF ELISA kit has been used in sandwich assays wherein a monoclonal antibody is used to capture the target VEGF antigen and a polyclonal antibody is used to detect the VEGF. Webb et al. supra (1998). See, also, e.g., Obermair et al., supra (1998). Keyt et al. J. Biol. Chem. 271:7788-7795 (1996); Keyt et al. J. Biol. Chem. 271:5638 (1996); and Shifren et al., supra (1996) also developed a colorimetric ELISA based on a dual monoclonal antibody pair. Although this ELISA was able to detect elevated VEGF levels in cancer patients, it lacked the sensitivity needed to measure endogenous levels of VEGF in normal individuals. Rodriguez et al. J. Immunol. Methods 219:45 (1998) described a two-site fluorimetric VEGF ELISA that yields a sensitivity of 10 pg/ml VEGF in neat plasma or serum. However, this fluorimetric assay only detects fully intact 165/165 and 165/110 species of VEGF (It has been reported that VEGF 165/165 can be proteolytically cleaved into three other forms: a 165/110 heterodimer, a 110/110 homodimer, and a 55-amino-acid C-terminal fragment (Keyt et al. J. Biol. Chem. 271:7788-7795 (1996); Keck et al. Arch. Biochem. Biophys. 344:103-113 (1997)).).

VEGF-A is a target for therapeutic antagonists such as e.g. AVASTIN® which bind to VEGF-A and blocks its activity. However, so far no assays described in literature or commercially available allows for the measurement of the VEGF-A level in the presence of such a VEGF-A antagonist. There is thus, an urgent need to develop assays and means to be used in these assays which allow for the detection of VEGF-A level in the presence of such an antagonist, in particular in the presence of such therapeutic antagonists.

### SUMMARY OF THE INVENTION

In a first aspect, the present invention relates to a method for measuring the level of VEGF-A in the presence of a VEGF-A antagonist, the method comprising: incubating a sample with a first and a second antibody wherein said first and said to second antibody both are capable of binding to VEGF-A in the presence of the VEGF-A antagonist, and detecting the complex formed, thereby measuring the level of VEGF-A in the presence of a VEGF-A antagonist. In embodiments, the binding of said first and of said second antibody does not interfere with each other. In embodiments, one of said antibodies is bound to or capable of binding to a solid phase and the other of said antibodies is detectably labeled. The detectably labeled complex formed comprises the first antibody, VEGF-A, and the second antibody.

In a second aspect, the present invention relates to a kit for measuring the level of VEGF-A in the presence of a VEGF-A antagonist, the kit comprising: a first and a second antibody wherein said first and said second antibody both are capable of binding to VEGF-A in the presence of the VEGF-A antagonist. In embodiments, the binding of said first and of said second antibody does not interfere with each other. In embodiments, one of said antibodies is bound to or capable of binding to a solid phase and the other of said antibodies is detectably labeled.

In a third aspect, the present invention relates to a composition of matter comprising a first and a second antibody wherein said first and said second antibody are both capable of binding to VEGF-A or a variant thereof, in the presence of an VEGF-A antagonist. In embodiments, the binding of said first and of said second antibody does not interfere with each other. In embodiments, one of said antibodies is bound to or capable of binding to a solid phase and the other of said antibodies is detectably labeled.

In a fourth aspect, the present invention relates to a method of detecting a complex comprising human VEGF-A and a non-human or chimeric protein comprising the steps of (a) incubating a sample comprising said complex with a detectably labeled antibody which is able to bind to or is binding to the human VEGF-A and/or the non-human or chimeric protein, and (b) detecting said detectably labeled antibody or antigen-binding fragment thereof.

### BRIEF DESCRIPTION OF THE SEVERAL VIEWS OF THE DRAWINGS

FIGS. 1A AND 1B: 1A: Assay Design; 1B: Assay Design Including VEGF-A Receptor.

FIG. 2: Amino Acid Sequences of the Light Chains and Heavy Chains of Antibodies M-13.2.5 and M-13.7.40; CDRs are highlighted in bold; FR are underlined.

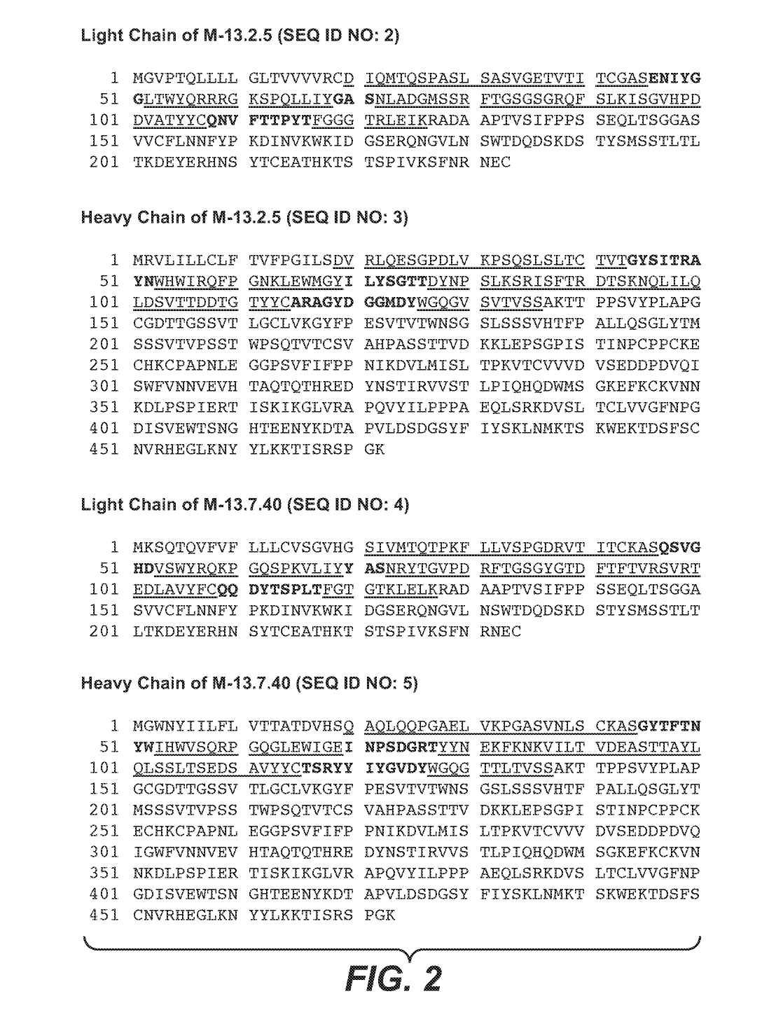

FIGS. 3A-3D: 3A-3D: Biacore Sensorgrams with and without VEGF-A receptor R1 or R2

### DETAILED DESCRIPTION OF THE INVENTION

### List of Sequences

SEQ ID NO: 1: human VEGF-A

SEQ ID NO: 2: Amino acid sequence of light chain of M-13.2.5

SEQ ID NO: 3: Amino acid sequence of heavy chain of M-13.2.5

SEQ ID NO: 4: Amino acid sequence of light chain of M-13.7.40

SEQ ID NO: 5: Amino acid sequence of heavy chain of M-13.7.40

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

The word “comprise”, and variations such as “comprises” and “comprising”, will be understood to imply the inclusion of a stated integer or step or group of integers or steps but not the exclusion of any other integer or step or group of integers or steps.

As used in this specification and the appended claims, the singular forms “a”, “an”, and “the” include plural referents, unless the content clearly dictates otherwise.

Concentrations, amounts, and other numerical data may be expressed or presented herein in a “range” format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “150 mg to 600 mg” should be interpreted to include not only the explicitly recited values of 150 mg to 600 mg, but to also include individual values and sub-ranges within the indicated range. Thus, included in this numerical range are individual values such as 150, 160, 170, 180, 190, . . . , 580, 590, 600 mg and sub-ranges such as from 150 to 200, 150 to 250, 250 to 300, 350 to 600, etc. This same principle applies to ranges reciting only one numerical value. Furthermore, such an interpretation should apply regardless of the breadth of the range or the characteristics being described.

The term “about” when used in connection with a numerical value is meant to encompass numerical values within a range having a lower limit that is 5% smaller than the indicated numerical value and having an upper limit that is 5% larger than the indicated numerical value.

The term “expression level” refers to the amount of gene product present in the body or a sample at a certain point of time. The expression level can e.g. be measured/quantified/detected by means of the protein or mRNA expressed from the gene. The expression level can for example be quantified by normalizing the amount of gene product of interest (e.g. mRNA or protein) present in a sample with the total amount of gene product of the same category (total protein or mRNA) in the same sample or a reference sample (e.g. a sample taken at the same time from the same individual or a part of identical size (weight, volume) of the same sample or by identifying the amount of gene product of interest per defined sample size (weight, volume, etc.). The expression level can be measured or detected by means of any method as known in the art, e.g. methods for the direct detection and quantification of the gene product of interest (such as mass spectrometry) or methods for the indirect detection and measurement of the gene product of interest that usually work via binding of the gene product of interest with one or more different molecules or detection means (e.g. primer(s), probes, antibodies, protein scaffolds) specific for the gene product of interest. The determination of the level of gene copies of gene product of interest comprising also the determination of the absence or presence of one or more fragments (e.g. via nucleic acid probes or primers, e.g. quantitative PCR, Multiplex ligation-dependent probe amplification (MLPA) PCR) is also within the knowledge of the skilled artisan.

In the context of the present invention, the term “peptide” refers to a short polymer of amino acids linked by peptide bonds. It has the same chemical (peptide) bonds as proteins, but is commonly shorter in length. The shortest peptide is a dipeptide, consisting of two amino acids joined by a single peptide bond. There can also be a tripeptide, tetrapeptide, pentapeptide, etc. Typically, a peptide has a length of up to 8, 10, 12, 15, 18 or 20 amino acids. A peptide has an amino end and a carboxyl end, unless it is a cyclic peptide.

In the context of the present invention, the term “polypeptide” refers to a single linear chain of amino acids bonded together by peptide bonds and typically comprises at least about 21 amino acids. A polypeptide can be one chain of a protein that is composed of more than one chain or it can be the protein itself if the protein is composed of one chain.

In the context of the different aspects of present invention, the term “protein” refers to a molecule comprising one or more polypeptides that resume a secondary and tertiary structure and additionally refers to a protein that is made up of several polypeptides, i.e. several subunits, forming quaternary structures. The protein has sometimes non-peptide groups attached, which can be called prosthetic groups or cofactors.

The term “complex” as used herein, refers to a whole that comprehends a number of individual components, parts or moieties which are in close proximity to each other and fulfil a common or interrelated function. The individual moieties of a complex may be of the same or of differing nature, i.e. they may be composed of the same, a similar or of differing chemical entity such as but not limited to nucleotides, amino acids, nucleic acids, peptides, polypeptides, proteins, carbohydrates, or lipids. Exemplified, a complex may comprise a number of associated proteins, or a mixture of one or more proteins and one or more nucleic acids or a mixture of one or more proteins and one or more lipids and/or carbohydrates. It is understood that any other combination of identical, similar or differing chemical entities is also encompassed. The individual moieties of a complex may or may not be interconnected. Typically, the individual parts of a complex are connected via covalent or non-covalent bonds. Exemplified, a complex may comprise a protein and the receptor, to which it is bound, or a complex may comprise a protein and an antibody bound to an epitope of said protein.

The term “antigen” refers to any substance that causes an immune system to produce antibodies against it. An antigen may originate from within the body (“self-antigen”) or from the external environment (“non-self”). Antigen presenting cells present antigens in the form of peptides on histocompatibility molecules. The T cells of the adaptive immune system recognize the antigens. Depending on the antigen and the type of the histocompatibility molecule, different types of T cells are activated.

An “epitope”, also known as “antigenic determinant”, is the segment of a macromolecule, in particular a segment of an antigen, which is recognized by the immune system, specifically by antibodies, B cells, or T cells. An epitope is typically part of an antigen and is capable of binding to an antibody or antigen-binding fragment thereof. In this context, the term “binding” preferably relates to a specific binding. In the context of the present invention the term “epitope” refers to the segment of protein that is recognized by an antibody. Epitopes usually consist of chemically active surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents.

As used herein, the term “variant” is to be understood as a polypeptide or polynucleotide which differs in comparison to the polypeptide or polynucleotide from which it is derived by one or more changes in its length or sequence. The polypeptide or polynucleotide from which a polypeptide or polynucleotide variant is derived is also known as the parent polypeptide or polynucleotide. The term “variant” comprises “fragments” or “derivatives” of the parent molecule. Typically, “fragments” are smaller in length or size than the parent molecule, whilst “derivatives” exhibit one or more differences in their sequence in comparison to the parent molecule. Also encompassed are modified molecules such as but not limited to post-translationally modified proteins (e.g. glycosylated, biotinylated, phosphorylated, ubiquitinated, palmitoylated, or proteolytically cleaved proteins) and modified nucleic acids such as methylated DNA. Also mixtures of different molecules such as but not limited to RNA-DNA hybrids, are encompassed by the term “variant”. Typically, a variant is constructed artificially, preferably by gene-technological means, whilst the parent protein or polynucleotide is a wild-type protein or polynucleotide, or a consensus sequence thereof. However, also naturally occurring variants are to be understood to be encompassed by the term “variant” as used herein. Further, the variants usable in the present invention may also be derived from homologs, orthologs, or paralogs of the parent molecule or from artificially constructed variant, provided that the variant exhibits at least one biological activity of the parent molecule, i.e. is functionally active.

In particular, the term “peptide variant”, “polypeptide variant”, “protein variant” is to be understood as a peptide, polypeptide, or protein which differs in comparison to the peptide, polypeptide, or protein from which it is derived by one or more changes in the amino acid sequence. The peptide, polypeptide, or protein, from which a peptide, polypeptide, or protein variant is derived, is also known as the parent peptide, polypeptide, or protein. Further, the variants usable in the present invention may also be derived from homologs, orthologs, or paralogs of the parent peptide, polypeptide, or protein or from artificially constructed variant, provided that the variant exhibits at least one biological activity of the parent peptide, polypeptide, or protein. The changes in the amino acid sequence may be amino acid exchanges, insertions, deletions, N-terminal truncations, or C-terminal truncations, or any combination of these changes, which may occur at one or several sites. A peptide, polypeptide, or protein variant may exhibit a total number of up to 50 (up to 1, 2, 3, 4, 5, 6, 7, 8, 9, 10, 15, 20, 25, 30, 35, 40, 45, or 50) changes in the amino acid sequence (i.e. exchanges, insertions, deletions, N-terminal truncations, and/or C-terminal truncations). The amino acid exchanges may be conservative, semi-conservative and/or non-conservative. Semi-conservative and especially conservative amino acid substitutions, wherein an amino acid is substituted with a chemically related amino acid are preferred. Typical substitutions are among the aliphatic amino acids, among the amino acids having aliphatic hydroxyl side chain, among the amino acids having acidic residues, among the amide derivatives, among the amino acids with basic residues, or the amino acids having aromatic residues. Typical semi-conservative and conservative substitutions are:

| Amino acid | Conservative substitution | Semi-conservative substitution |
|---|---|---|
| A | G; S; T | N; V; C |
| C | A; V; L | M; I; F; G |
| D | E; N; Q | A; S; T; K; R; H |
| E | D; Q; N | A; S; T; K; R; H |
| F | W; Y; L; M; H | I; V; A |
| G | A | S; N; T; D; E; N; Q |
| H | Y; F; K; R | L; M; A |
| I | V; L; M; A | F; Y; W; G |
| K | R; H | D; E; N; Q; S; T; A |
| L | M; I; V; A | F; Y; W; H; C |
| M | L; I; V; A | F; Y; W; C, |
| N | Q | D; E; S; T; A; G; K; R |
| P | V; I | L; A; M; W; Y; S; T; C; F |
| Q | N | D; E; A; S; T; L; M; K; R |
| R | K; H | N; Q; S; T; D; E; A |
| S | A; T; G; N | D; E; R; K |
| T | A; S; G; N; V | D; E; R; K; I |
| V | A; L; I | M; T; C; N |
| W | F; Y; H | L; M; I; V; C |
| Y | F; W; H | L; M; I; V; C |

Changing from A, F, H, I, L, M, P, V, W or Y to C is semi-conservative if the new cysteine remains as a free thiol. Furthermore, the skilled person will appreciate that glycines at sterically demanding positions should not be substituted and that P should not be introduced into parts of the protein which have an alpha-helical or a beta-sheet structure.

Alternatively or additionally, a “variant” as used herein, can be characterized by a certain degree of sequence identity to the parent peptide, polypeptide, or protein from which it is derived. More precisely, a peptide, polypeptide, or protein variant in the context of the present invention exhibits at least 80% sequence identity to its parent peptide, polypeptide, or protein. In particular, the variant may exhibit a sequence identity of at least 85%, 90%, 95%, 97%, or 99%. The sequence identity of peptide, polypeptide, or protein variants is over a continuous stretch of 20, 30, 40, 45, 50, 60, 70, 80, 90, 100 or more amino acids.

The term “biomarker” or “indicator” are used interchangeably herein. In the context of present invention, a biomarker can be defined as a substance within a biological system that is used as an indicator of a biological state of said system. In the art, the term “biomarker” is sometimes also applied to means for the detection of said endogenous substances (e.g. antibodies, nucleic acid probes etc., imaging systems). In the context of present invention, the term “biomarker” shall be only applied for the substance, not for the detection means. Thus, biomarkers can be any kind of molecule present in a living organism, such as a nucleic acid (DNA, mRNA, miRNA, rRNA etc.), a protein (cell surface receptor, cytosolic protein etc.), a metabolite or hormone (blood sugar, insulin, estrogen, etc.), a molecule characteristic of a certain modification of another molecule (e.g. sugar moieties or phosphoryl residues on proteins, methyl-residues on genomic DNA) or a substance that has been internalized by the organism or a metabolite of such a substance.

The term “VEGF” refers to VEGFs from human and non-human species such as mouse, rat or primate. Sometimes the VEGF from a specific species are indicated by terms such as hVEGF for human VEGF, mVEGF for murine VEGF, etc.

“VEGF biological activity” includes but is not limited to the binding of VEGF to any VEGF receptor, and VEGF signaling activity such as regulation of both normal and abnormal angiogenesis and vasculogenesis (Ferrara and Davis-Smyth (1997) Endocrine Rev. 18:4-25; Ferrara (1999) J. Mol. Med. 77:527-543); promoting embryonic vasculogenesis and angiogenesis (Carmeliet et al. (1996) Nature 380:435-439; Ferrara et al. (1996) Nature 380:439-442); and modulating the cyclical blood vessel proliferation in the female reproductive tract and for bone growth and cartilage formation (Ferrara et al. (1998) Nature Med. 4:336-340; Gerber et al. (1999) Nature Med. 5:623-628). In addition to being an angiogenic factor in angiogenesis and vasculogenesis, VEGF, as a pleiotropic growth factor, exhibits multiple biological effects in other physiological processes, such as endothelial cell survival, vessel permeability and vasodilation, monocyte chemotaxis and calcium influx (Ferrara and Davis-Smyth (1997), supra and Cebe-Suarez et al. Cell. Mol. Life Sci. 63:601-615 (2006)). Moreover, recent studies have reported mitogenic effects of VEGF on a few non-endothelial cell types, such as retinal pigment epithelial cells, pancreatic duct cells, and Schwann cells. Guerrin et al. (1995) J. Cell Physiol. 164:385-394; Oberg-Welsh et al. (1997) Mol. Cell. Endocrinol. 126:125-132; Sondell et al. (1999) J. Neurosci. 19:5731-5740.

In the context of the present invention, “VEGF-A” refers to vascular endothelial growth factor protein A, exemplified by SEQ ID NO: 1 (Swiss Prot Accession Number P15692, Gene ID (NCBI): 7422). VEGF-A may form disulfide linked homodimer and plays a role as glycosylated mitogen that specifically acts on endothelial cells and has various effects, including mediating increased vascular permeability, inducing angiogenesis, vasculogenesis and endothelial cell growth, promoting cell migration, and inhibiting apoptosis. The term “VEGF-A” encompasses the protein having the amino acid sequence of SEQ ID NO: 1 as well as, isoforms, fragments and variants thereof. Alternatively spliced transcripts, encoding either freely secreted or cell-associated isoforms, have been characterized, e.g. splice isoforms of VEGF-A, e.g., VEGF₁₂₁, VEGF₁₄₅, VEGF₁₆₅, VEGF₁₈₉ and VEGF₂₀₆, together with the naturally occurring allelic and processed forms thereof. Fragments include but are not limited to the 110-amino acid human vascular endothelial cell growth factor generated by plasmin cleavage of VEGF₁₆₅ as described in Ferrara Mol. Biol. Cell 21:687 (2010) and Leung et al. Science 246:1306 (1989), and Houck et al. Mol. Endocrin. 5:1806 (1991). The term “VEGF-A” thus relates to the protein having the amino acid sequence of SEQ ID NO:1 as well as to the splice isoforms VEGF₁₂₁, VEGF₁₄₅, VEGF₁₆₅, VEGF₁₈₉ and VEGF₂₀₆, to the 110-amino acid fragment thereof, as well as to variants of the amino acid sequence of SEQ ID NO:1, variants of the splice isoforms VEGF₁₂₁, VEGF₁₄₅, VEGF₁₆₅, VEGF₁₈₉ and VEGF₂₀₆, and variants of the 110-amino acid fragment thereof.

The two best characterized VEGF receptors are “VEGFR1” (also known as Flt-1) and “VEGFR2” (also known as KDR and FLK-1 for the murine homolog). The specificity of each receptor for each VEGF family member varies but VEGF-A binds to both Flt-1 and KDR. The full length Flt-1 receptor includes an extracellular domain that has seven Ig domains, a transmembrane domain, and an intracellular domain with tyrosine kinase activity. The extracellular domain is involved in the binding of VEGF and the intracellular domain is involved in signal transduction. VEGF-A receptor molecules, or fragments thereof, that specifically bind to VEGF-A can be used as VEGF-A inhibitors that bind to and sequester the VEGF-A protein, thereby preventing it from signaling. Also the soluble form of the receptor exerts an inhibitory effect on the biological activity of the VEGF-A protein by binding to VEGF-A, thereby preventing it from binding to its natural receptors present on the surface of target cells.

The term “disease” and “disorder” are used interchangeably herein, referring to an abnormal condition, especially an abnormal medical condition such as an illness or injury, wherein a tissue, an organ or an individual is not able to efficiently fulfil its function anymore. Typically, but not necessarily, a disease is associated with specific symptoms or signs indicating the presence of such disease. The presence of such symptoms or signs may thus, be indicative for a tissue, an organ or an individual suffering from a disease. An alteration of these symptoms or signs may be indicative for the progression of such a disease. A progression of a disease is typically characterised by an increase or decrease of such symptoms or signs which may indicate a “worsening” or “bettering” of the disease. The “worsening” of a disease is characterised by a decreasing ability of a tissue, organ or organism to fulfil its function efficiently, whereas the “bettering” of a disease is typically characterised by an increase in the ability of a tissue, an organ or an individual to fulfil its function efficiently. A tissue, an organ or an individual being at “risk of developing” a disease is in a healthy state but shows potential of a disease emerging. Typically, the risk of developing a disease is associated with early or weak signs or symptoms of such disease. In such case, the onset of the disease may still be prevented by treatment. Examples of a disease include but are not limited to traumatic diseases, inflammatory diseases, infectious diseases, cutaneous conditions, endocrine diseases, intestinal diseases, neurological disorders, joint diseases, genetic disorders, autoimmune diseases, and various types of cancer.

The terms “cell proliferative disorder” and “proliferative disorder” refer to disorders that are associated with some degree of abnormal cell proliferation.

The term “tumor”, as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder” and “tumor” are not mutually exclusive as referred to herein.

The terms “cancer” and “cancerous” refer to or describe the physiological condition in mammals that is typically characterized by unregulated cell proliferation. Examples of cancer include but are not limited to, carcinoma, lymphoma, blastoma, sarcoma, and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including, e.g., gastrointestinal cancer), pancreatic cancer (including, e.g., metastic pancreatic cancer), glioblastoma, cervical cancer, ovarian cancer, liver cancer, bladder cancer, hepatoma, breast cancer (including locally advanced, recurrent or metastatic HER-2 negative breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NEIL; intermediate grade/follicular NHL; intermediate grade diffuse NHL; high grade immunoblastic NHL; high grade lymphoblastic NHL; high grade small non-cleaved cell NEIL; bulky disease NEIL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

“Symptoms” of a disease are implication of the disease noticeable by the tissue, organ or organism having such disease and include but are not limited to pain, weakness, tenderness, strain, stiffness, and spasm of the tissue, an organ or an individual. “Signs” or “signals” of a disease include but are not limited to the change or alteration such as the presence, absence, increase or elevation, decrease or decline, of specific indicators such as biomarkers or molecular markers, or the development, presence, or worsening of symptoms. Symptoms of pain include, but are not limited to an unpleasant sensation that may be felt as a persistent or varying burning, throbbing, itching or stinging ache.

As used herein, a “patient” means any mammal, fish, reptile or bird that may benefit from the prognosis or treatment disclosed herein. In particular, a “patient” is selected from the group consisting of laboratory animals (e.g. mouse, rat, rabbit, or zebrafish), domestic animals (including e.g. guinea pig, rabbit, horse, donkey, cow, sheep, goat, pig, chicken, camel, cat, dog, turtle, tortoise, snake, lizard or goldfish), or primates including chimpanzees, bonobos, gorillas and human beings. In particular the “patient” is a human being.

The term “sample” or “sample of interest” are used interchangeably herein, referring to a part or piece of a tissue, organ or individual, typically being smaller than such tissue, organ or individual, intended to represent the whole of the tissue, organ or individual. Upon analysis a sample provides information about the tissue status or the health or diseased status of an organ or an individual. Examples of samples include but are not limited to fluid samples such as blood, serum, plasma, synovial fluid, urine, saliva, and lymphatic fluid, or solid samples such as tissue extracts. Analysis of a sample may be accomplished on a visual or chemical basis. Visual analysis includes but is not limited to microscopic imaging or radiographic scanning of a tissue, organ or individual allowing for morphological evaluation of a sample. Chemical analysis includes but is not limited to the detection of the presence or absence of specific indicators or alterations in their amount or level.

The term “reference sample” as used herein, refers to a sample which is analysed in a substantially identical manner as the sample of interest and whose information is compared to that of the sample of interest. A reference sample thereby provides a standard allowing for the evaluation of the information obtained from the sample of interest. A reference sample may be derived from a healthy or normal tissue, organ or individual, thereby providing a standard of a healthy status of a tissue, organ or individual. Differences between the status of the normal reference sample and the status of the sample of interest may be indicative of the risk of disease development or the presence or further progression of such disease or disorder. A reference sample may also be derived from the same tissue, organ, or individual as the sample of interest but has been taken at an earlier time point. Differences between the status of the earlier taken reference sample and the status of the sample of interest may be indicative of the progression of the disease, i.e. a bettering or worsening of the disease over time. A reference sample was taken at an earlier or later time point in case a period of time has lapsed between taking of the reference sample and taking of the sample of interest. Such period of time may represent months (1, 2, 3, 4, 5, 6, 7, 8, 9, 10, 11, 12 months), weeks (e.g. 1, 2, 3, 4, 5, 6, 7, 8 weeks), days (e.g. 1, 2, 3, 4, 5, 10, 15, 20, 25, 30, 35, 40, 45, 50, 60, 70, 80, 90, 100, 150, 200, 250, 300, 350days), or hours (1, 2, 3, 4, 5, 6, 7, 8, 9, 10, 11, 12 hours).

The terms “lowered” or “decreased” level of an indicator refer to the level of such indicator in the sample being reduced in comparison to the reference or reference sample. The terms “elevated” or “increased” level of an indicator refers to the level of such indicator in the sample being higher in comparison to the reference or reference sample.

The term “agonist” as used herein refers to a substance that causes an action in a tissue, organ or individual such as receptor-signaling, gene expression, protein synthesis, and protein degradation. Typically, agonists act by binding to the active site or to allosteric sites of a receptor molecule thereby, triggering a specific reaction. Examples for agonists include but are not limited to nucleic acid molecules, such as mRNA or miRNA, or proteins, such as hormones, cytokines, growth factors, neurotransmitters, and transcription factors.

The term “antagonist” as used herein refers to a substance blocking the action of an agonist. Typically, antagonists act by binding to the active site or to allosteric sites of a receptor molecule, or interact with unique binding sites not normally involved in the regulation of the activity of the receptor. Typically, an antagonist competes with the agonist at structurally-defined binding sites or alters the binding site of the agonist in a manner that the agonist is not able to cause the action it would normally cause due to its binding. The antagonist activity may be reversible or irreversible depending on the longevity of the interaction of the antagonist-receptor complex.

Examples for antagonists include but are not limited to nucleic acid molecules, such as siRNAs or miRNAs, or proteins such as hormones, cytokines, growth factors or neurotransmitter, antibodies, or transcription factors.

The term “antagonistic antibody” refers to an antibody which partially or fully decreases or completely prevents at least one functional activity of the molecule of interest (e.g. the peptide, polypeptide or protein of interest). Typically, an antagonistic antibody binds to the active site of a receptor and thereby prevents the binding of the agonist to the receptor, or binds to the receptor at a different site in a manner which sterically hinders binding of the agonist to the receptor.

The term “receptor” as used herein refers to a molecule such as a protein or polynucleotide, to which one or more specific signaling molecules bind. Signaling molecules may act as agonist or antagonist including without limitation nucleic acid molecules, such as siRNAs or miRNAs, or proteins such as hormones, cytokines, growth factors or neurotransmitter, antibodies or transcription factors. Receptors may be localised at the plasma membrane of a cell, within the cytoplasm and/or in intracellular compartments.

The term “immunoglobulin (Ig)” as used herein refers to immunity conferring glycoproteins of the immunoglobulin superfamily. “Surface immunoglobulins” are attached to the membrane of effector cells by their transmembrane region and encompass molecules such as but not limited to B-cell receptors, T -cell receptors, class I and II major histocompatibility complex (MHC) proteins, beta-2 microglobulin (˜2M), CD3, CD4 and CDS.

Typically, the term “antibody” as used herein refers to secreted immunoglobulins which lack the transmembrane region and can thus, be released into the bloodstream and body cavities. Human antibodies are grouped into different isotypes based on the heavy chain they possess. There are five types of human Ig heavy chains which define the class of antibody, i.e. defining IgA, IgD, IgE, IgG, and IgM antibodies, each performing different roles, and directing the appropriate immune response against different types of antigens. IgA is found in mucosal areas, such as the gut, respiratory tract and urogenital tract, as well as in saliva, tears, and breast milk and prevents colonization by pathogens (Underdown & Schiff (1986) Annu. Rev. Immunol. 4:389-417). IgD mainly functions as an antigen receptor on B cells that have not been exposed to antigens and is involved in activating basophils and mast cells to produce antimicrobial factors (Geisberger et al. (2006) Immunology 118:429-437; Chen et al. (2009) Nat. Immunol. 10:889-898). IgE is involved in allergic reactions via its binding to allergens triggering the release of histamine from mast cells and basophils. IgE is also involved in protecting against parasitic worms (Pier et al. (2004) Immunology, Infection, and Immunity, ASM Press). IgG provides the majority of antibody-based immunity against invading pathogens and is the only antibody isotype capable of crossing the placenta to give passive immunity to fetus (Pier et al. (2004) Immunology, Infection, and Immunity, ASM Press). In humans there are four different IgG subclasses (IgG1, 2, 3, and 4), named in order of their abundance in serum with IgG1 being the most abundant (˜66%), followed by IgG2 (˜23%), IgG3 (˜7%) and IgG (˜4%). The biological profile of the different IgG classes is determined by the structure of the respective hinge region. IgM is expressed on the surface of B cells in a monomeric form and in a secreted pentameric form with very high avidity. IgM is involved in eliminating pathogens in the early stages of B cell mediated (humoral) immunity before sufficient IgG is produced (Geisberger et al. (2006) Immunology 118:429-437). Antibodies are not only found as monomers but are also known to form dimers of two Ig units (e.g. IgA), tetramers of four Ig units (e.g. IgM of teleost fish), or pentamers of five Ig units (e.g. mammalian IgM).

Antibodies are typically made of four polypeptide chains comprising two identical heavy chains and identical two light chains which are connected via disulfide bonds and resemble a “Y”-shaped macro-molecule. Each of the chains comprises a number of immunoglobulin domains out of which some are constant domains and others are variable domains. Immunoglobulin domains consist of a 2-layer sandwich of between 7 and 9 antiparallel strands arranged in two β-sheets. Typically, the heavy chain of an antibody comprises four Ig domains with three of them being constant (CH domains: CH1, CH2, CH3) domains and one of the being a variable domain (VH). The light chain typically comprises one constant Ig domain (CL) and one variable Ig domain (VL). Exemplified, the human IgG heavy chain is composed of four Ig domains linked from N- to C-terminus in the order VL1-CH1-CH2-CH3, whereas the human IgG light chain is composed of two immunoglobulin domains linked from N- to C-terminus in the order VL-CL, being either of the kappa or lambda type (Vkappa-Ckappa or Vlambda.-Clambda.). Exemplified, the constant chain of human IgG comprises 447 amino acids.

The term “hypervariable region (HVR or HV)” and “complementary determining region (CDR)” are used interchangeably herein and refers to the regions of an antibody-variable domain which are hypervariable in sequence and/or form structurally defined loops. Generally, antibodies comprise six HVRs or CDRs; three in VH (H1, H2, H3), and three in VL (L1, L2, L3). In native antibodies, H3 and L3 display the most diversity of the six HVRs, and H3 in particular is believed to play a unique role in conferring fine specificity to antibodies. See, e.g., Xu et al. Immunity 13:37-45 (2000); Johnson and Wu in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993) and Sheriff et al., Nature Struct. Biol. 3:733-736 (1996). A number of HVR delineations are in use and are encompassed herein. The HVRs that are Kabat complementarity-determining regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat CDRs and Chothia structural loops, and are used by Oxford Molecular's AbM antibody-modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat |
| | | | | Numbering) |
| H1 | H31-H35 | H26-H35 | H26-H32 | H30-H35 (Chothia |
| | | | | Numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (H2), and 93-102, 94-102, or 95-102 (H3) in the VH. The variable-domain residues are numbered according to Kabat et al., supra, for each of these extended-HVR definitions.

Amino acids of the “Framework region” or “FR” are residues which are less variable than the HVR residues as herein defined. Typically, four FR separate the three HVRs and form a beta-sheet structure which serves as a scaffold to hold the HV region in position to contact antigen.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The “EU index as in Kabat” refers to the residue numbering of the human IgG lEU antibody. Accordingly, CH domains in the context of IgG are as follows: “CHI” refers to amino acid positions 118-220 according to the EU index as in Kabat; “CH2” refers to amino acid positions 237-340 according to the EU index as in Kabat; and “CH3” refers to amino acid positions 341-447 according to the EU index as in Kabat.

The term “binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including but not limited to surface plasmon resonance based assay (such as the BIAcore assay as described in PCT Application Publication No. WO2005/012359); enzyme-linked immunoabsorbent assay (ELISA); and competition assays (e.g. RIA' s). Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative and exemplary embodiments for measuring binding affinity are described in the following.

The “Kd” or “Kd-value” may be measured by using surface-plasmon resonance assays using a BIACORE®-2000 or a BIACORE®-3000 instrument (BIAcore, Inc., Piscataway, N.J.) at 25° C. with immobilized antigen CM5 chips at ˜10 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately ten response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% TWEEN 20™ surfactant (PBST) at 25° C. at a flow rate of approximately 25 μl/min. Association rates (kₒₙ) and dissociation rates (k_{off}) are calculated using a simple one-to-one Langmuir binding model (BIAcore® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹s⁻¹ by the surface-plasmon resonance assay above, then the on-rate can be determined by using a fluorescent quenching technique that measures the increase or decrease in fluorescence-emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow-equipped spectrophotometer (Aviv Instruments) or a 8000-series SLM-AMINCO™ spectrophotometer (ThermoSpectronic) with a stirred cuvette.

An “on-rate,” “rate of association,” “association rate,” or “kₒₙ” can also be determined as described above using a BIACORE®-2000 or a BIACORE®-3000 system (BIAcore, Inc., Piscataway, N.J.).

Typically, antibodies bind with a sufficient binding affinity to their target, for example, with a K_{d} value of between 500 nM-1 pM, i.e. 500 nM, 450 nM, 400 nM, 350 nM, 300 nM, 250 nM, 200 nM, 150 nM, 100 nM, 50 nM, 1 nM, 900 pM, 800 pM, 700 pM, 600 pM, 500 pM, 400 pM, 300 pM, 200 pM, 100 pM, 50 pM, 1 pM.

An “affinity-matured” antibody is one with one or more alterations in one or more HVRs thereof which result in an improvement in the affinity of the antibody for antigen, compared to a parent antibody which does not possess those alteration(s). An affinity-matured antibody has nanomolar or even picomolar affinities for the target antigen. Affinity-matured antibodies are produced by procedures known in the art. For example, Marks et al., Bio/Technology 10:779-783 (1992) describes affinity maturation by VH- and VL-domain shuffling. Random mutagenesis of HVR and/or framework residues is described by, for example: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

The term antibody also encompasses “antigen-binding fragment” thereof. The term “antigen-binding fragments” of an antibody refers to molecules which possess the ability to bind to an antigen in a similar fashion as an antibody but which is smaller in size than a complete antibody molecule. Exemplified, three “antigen binding fragments” of an antibody are obtained by papain digestion which produces three fragments, namely two identical fragments, called “Fab fragments” (also referred to as “Fab portion” or “Fab region”) each with a single antigen binding site, and a residual “Fc fragment” (also referred to as “Fc portion” or “Fc region”) whose name reflects its ability to crystallize readily. In IgG, IgA and IgD isotypes, the Fc region is composed of two identical protein fragments, derived from the CH2 and CH3 domains of the antibody's two heavy chains; in IgM and IgE isotypes, the Fc regions contain three heavy chain constant domains (CH2-4) in each polypeptide chain. In addition, further antigen binding fragments occur naturally or have been constructed artificially. The term “Fab′ fragment” refers to a Fab fragment additionally comprise the hinge region of an Ig molecule whilst “F(ab′)2 fragments” are understood to comprise two Fab′ fragments being either chemically linked or connected via a disulfide bond. Whilst “single domain antibodies (sdAb)” (Desmyter et al. (1996) Nat. Structure Biol. 3:803-811) and “Nanobodies” only comprise a single VH domain, “single chain Fv (scFv)” fragments comprise the heavy chain variable domain joined via a short linker peptide to the light chain variable domain (Huston et al. (1988) Proc. Natl. Acad. Sci. USA 85, 5879-5883). Divalent single-chain variable fragments (di-scFvs) can be engineered by linking two scFvs (scFvA-scFvB). This can be done by producing a single peptide chain with two VH and two VL regions, yielding “tandem scFvs” (VHA-VLA-VHB-VLB). Another possibility is the creation of scFvs with linkers that are too short for the two variable regions to fold together, forcing scFvs to dimerize. Usually linkers with a length of 5 residues are used to generate these dimers. This type is known as “diabodies”. Still shorter linkers (one or two amino acids) between a VH and VL domain lead to the formation of monospecific trimers, so-called “triabodies” or “tribadies”. Bispecific diabodies are formed by expressing to chains with the arrangement VHA-VLB and VHB-VLA or VLA-VHB and VLB-VHA, respectively. Singlechain diabodies (scDb) comprise a VHA-VLB and a VHB-VLA fragment which are linked by a linker peptide (P) of 12-20 amino acids, preferably 14 amino acids, (VHA-VLB-P-VHB-VLA). “Bi-specific T-cell engagers (BiTEs)” are fusion proteins consisting of two scFvs of different antibodies wherein one of the scFvs binds to T cells via the CD3 receptor, and the other to a tumor cell via a tumor specific molecule (Kufer et al. (2004) Trends Biotechnol. 22:238-244). Dual affinity retargeting molecules (“DART” molecules) are diabodies additionally stabilized through a C-terminal disulfide bridge.

The terms “antibody” and “antigen binding fragment thereof” also encompasses variants of the antibody or variants of the antigen binding fragment thereof. As for any protein variant as defined above, also the variant of an antibody or the variant of an antigen binding fragment thereof, is to be understood as an antibody or an antigen binding fragment, which differs in comparison to the antibody, or antigen binding fragment, from which it is derived by one or more changes in its length or sequence as defined in detail above with regard to protein variants. In addition, an antibody variant or the variant of an antigen-binding fragment thereof may exhibit different degrees of sequence identity in different parts of the antibody or antigen-binding fragment. With regard to the framework regions, a certain degree of variability is envisaged herein, i.e. the individual FRs can comprise or consist of the specifically recited amino acid sequence or of an amino acid sequence at least 80%, at least 85%, at least 90%, at least 92.5%, at least 95%, at least 98%, at least 99% or at least 99.5%. It will be appreciated that for different FRs, a different degree of sequence identity may be allowable, depending on the actual sequence and e.g. the length of the respective FR sequence, as well as its location within the respective variable chain domain. However, in an antibody variant or variant of an antigen-binding fragment thereof, the CDRs may either have the specifically recited sequence of said CDR, or may differ therefrom in at most one amino acid substitution. As such, one amino acid in each of the CDRs can be replaced by a different amino acid. It will be appreciated that an amino acid substitution may be present in some, but not all CDRs of one chain of one antibody.

Antibodies or antigen-binding fragments thereof, or their variants, may be “detectably labeled”. The term “detectably labeled” encompasses labels that can be directly or indirectly detected. Suitable labels include but are not limited to molecules detectable by spectroscopic, photochemical, biochemical, immunochemical, chemical, or other physical means. Suitable labels include but are not limited to fluorescent dye (e.g. GFT and its variants, FITC, TRITC, fluorescein and rhodamine, and the like), electron-dense reagent (e.g. gold), enzymes (e.g., as commonly used in an ELISA), radionuclide containing molecules (i.e., radioisotopes), chemiluminescent molecules, electrochemiluminescent molecules, biotin, digoxigenin, or hapten and other entities which are or can be made detectable. Exemplified an antibody may be biotinylated or ruthenylated. Methods for labeling of an antibody are well-known to the person skilled in the art and abundantly described e.g. in Haugland (2003) Molecular Probes Handbook of Fluorescent Probes and Research Chemicals, Molecular Probes, Inc.; Brinkley (1992) Bioconjugate Chem. 3:2; Garman, (1997) Non-Radioactive Labeling: A Practical Approach, Academic Press, London; Means (1990) Bioconjugate Chem. 1:2; Glazer et al Chemical Modification of Proteins. Laboratory Techniques in Biochemistry and Molecular Biology (T. S. Work and E. Work, Eds.) American Elsevier Publishing Co., New York; Lundblad, R. L. and Noyes, C. M. (1984) Chemical Reagents for Protein Modification, Vols. I and II, CRC Press, New York; Pfleiderer, G. (1985) “Chemical Modification of Proteins”, Modern Methods in Protein Chemistry, H. Tschesche, Ed., Walter DeGruyter, Berlin and New York; and Wong (1991) Chemistry of Protein Conjugation and Cross-linking, CRC Press, Boca Raton, Fla.); DeLeon-Rodriguez et al, Chem. Eur. J. 10 (2004) 1149-1155; Lewis et al, Bioconjugate Chem. 12 (2001) 320-324; Li et al, Bioconjugate Chem. 13 (2002) 110-115; Mier et al Bioconjugate Chem. 16 (2005) 240-237.

Antibodies target potential biomarker whose presence, absence, or level may be detected via various measurement methods, in particular immunoassay techniques. These include but are not limited to Western blotting (with or without immunoprecipitation), 2-dimensional SDS-PAGE, immunoprecipitation, fluorescence activated cell sorting (FACS), flow cytometry, and immuno assay procedures. Said methods allow for assaying a wide variety of tissues and samples, including plasma or serum. A wide range of immunoassay techniques using such an assay format are available, see, e.g., U.S. Pat. Nos. 4,016,043, 4,424,279, and 4,018,653. These include both single-site and two-site or “sandwich” assays of the non-competitive types, as well as in the traditional competitive binding assays. These assays also include direct binding of a labeled antibody to a target biomarker.

“Sandwich assays” are among the most useful and commonly used assays encompassing a number of variations of the sandwich assay technique. Briefly, in a typical assay, an unlabeled antibody is immobilized on a solid substrate, and the sample to be tested brought into contact with the bound molecule. After a suitable period of incubation, for a period of time sufficient to allow formation of an antibody-antigen complex, a second antibody labeled with a reporter molecule capable of producing a detectable signal is then added and incubated, allowing time sufficient for the formation of another complex of antibody-antigen-labeled antibody. Any unreacted material may be washed away, and the presence of the analyte is determined by observation of a signal produced by the reporter molecule. The results may either be qualitative, by simple observation of the visible signal, or may be quantitated by comparison with a control sample containing known amounts of biomarker.

Variations on the sandwich assay include a simultaneous assay, in which both, sample to be analyzed and labeled antibody are added simultaneously to an immobilized antibody. These techniques are well-known to those skilled in the art, including any minor variations as will be readily apparent. In a typical sandwich assay, a first antibody to a first epitope on the biomarker is either covalently or passively bound to a solid phase. The solid phase is typically glass or a polymer, the most commonly used polymers being cellulose, polyacrylamide, nylon, polystyrene, polyvinyl chloride, or polypropylene. The solid phase may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be analyzed is then added to the solid phase complex and incubated for a period of time sufficient (e.g. 2-40 minutes or overnight if more convenient) and under suitable conditions (e.g., from room temperature to 40° C., such as between 25° C. and 32° C.) to allow binding of any subunit present in the antibody. Following the incubation period, the antibody subunit solid phase is washed and incubated with a second antibody specific for a different epitope on the biomarker. The second antibody is linked to a reporter molecule which is used to indicate the binding of the second antibody to the analyte.

An alternative, competitive method involves immobilizing the analyte on a solid phase and then exposing the immobilized target together with the sample to be analyzed to an antibody specific to the analyte, which may or may not be labeled with a reporter molecule. Depending on the amount of target molecule in the sample and the strength of the reporter molecule signal, a competition by the target molecule may be detectable directly via such labeled antibody. Alternatively, a second labeled antibody, specific to the first antibody is exposed to the target-first antibody complex to form a target-first antibody-second antibody tertiary complex. The complex is detected by the signal emitted by the reporter molecule.

The terms “reporter molecule” or “directly detectable label”, as used in the present specification, refer to a molecule which, by its chemical nature, provides an analytically identifiable signal which allows the detection of antigen-bound antibody. The most commonly used reporter molecules in this type of assay are either enzymes, fluorophores or radionuclide containing molecules (i.e., radioisotopes) and chemiluminescent or electrochemiluminescent molecules.

In the case of an “enzyme immunoassay (EIA)”, an enzyme is conjugated to the second antibody, typically by means of glutaraldehyde or periodate. As will be readily recognized, however, a wide variety of different conjugation techniques exist, which are readily available to the skilled artisan. Commonly used enzymes include but are not limited to horseradish peroxidase, glucose oxidase, beta-galactosidase, and alkaline phosphatase, amongst others. The substrates to be used with the specific enzymes are generally chosen for the production, upon hydrolysis by the corresponding enzyme, of a detectable color change. Examples of suitable enzymes include but are not limited to alkaline phosphatase and peroxidase. It is also possible to employ fluorogenic substrates, which yield a fluorescent product rather than the chromogenic substrates noted above. In all cases, the enzyme-labeled antibody is added to the first antibody-molecular marker complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually but not necessarily spectrophotometrically, to give an indication of the amount of analyte which is present in the sample. Alternately, fluorescent compounds, such as fluorescein and rhodamine, may be chemically coupled to antibodies without altering their binding capacity. When activated by illumination with light of a particular wavelength, the fluorochrome-labeled antibody adsorbs the light energy, inducing a state to excitability in the molecule, followed by emission of the light at a characteristic color visually detectable with a light microscope. As in the EIA, the fluorescent labeled antibody is allowed to bind to the first antibody-molecular marker complex. After washing off the unbound reagent, the remaining tertiary complex is then exposed to the light of the appropriate wavelength, the fluorescence observed indicates the presence, absence, or level of the analyte. Immunofluorescence and EIA techniques are both very well established in the art. However, other reporter molecules, such as radioisotope, chemiluminescent, electrochemiluminescent, or bioluminescent molecules, may also be employed. Immunoassays for detecting VEGF are described in, e.g., U.S. Pat. Nos. 6,855,508 and 7,541,160 and U.S. Patent Publication No. 2010/0255515. Suitable platforms for detecting VEGF are described in, e.g., EP 0939319 and EP 1610129.

Further, the mRNAs or DNAs of an analyte of interest may be detected by a method selected from the group consisting of using Northern, dot-blot, or polymerase chain reaction (PCR) analysis, array hybridization, RNase protection assay, or using DNA microarrays, which are commercially available, including DNA microarray snapshots. For example, real-time PCR (RT-PCR) assays such as quantitative PCR assays are well known in the art. Methods of detecting the mRNA of an analyte of interest in a biological sample include but are not limited to producing cDNA from the sample by reverse transcription using at least one primer; amplifying the cDNA so produced; and detecting the presence of the amplified cDNA. In addition, such methods may include one or more steps which allow for the determination of the levels of mRNA in a biological sample (e.g., by simultaneously examining the levels a comparative control mRNA sequence of a “housekeeping” gene such as an actin family member). Additionally or alternatively, the sequence of the amplified cDNA may be determined. Northern blot analysis is a conventional technique well known in the art and is described, for example, in Molecular Cloning, a Laboratory Manual, second edition, 1989, Sambrook, Fritch, Maniatis, Cold Spring Harbor Press, 10 Skyline Drive, Plainview, N.Y. 11803-2500. Typical protocols for evaluating the status of genes and gene products are found, for example in Ausubel et al. eds., 1995, Current Protocols In Molecular Biology, Units 2 (Northern Blotting), 4 (Southern Blotting), 15 (Immunoblotting) and 18 (PCR Analysis).

A “particle” as used herein means a small, localized object to which can be ascribed a physical property such as volume, mass or average size. Microparticles may accordingly be of a symmetrical, globular, essentially globular or spherical shape, or be of an irregular, asymmetric shape or form. The size of a particle envisaged by the present invention may vary. Typically, microparticles are with a diameter in the nanometer and micrometer range. Microparticles may have a diameter of 50 nanometers to 50 micrometers, i,e. 50 nm, 100 nm, 200 nm, 300 nm, 400 nm, 500 nm, 600 nm, 700 nm, 800 nm, 900 nm, 1000 nm (i.e. 1 μm), 2 μm, 3 μm, 4 μm, 5 μm, 6 μm, 7 μm, 8 μm, 9 μm, 10 μm, 15 μm, 20 μm, 25 μm, 30 μm, 35 μm, 40 μm, 45 μm, and 50 μm. In particular, microparticles have a diameter of between 100 nm and 10 μm, in particular of 200 nm to 5 μm or of 750 nm to 5 μm. Microparticles comprise or consist of any suitable material known to the person skilled in the art, e.g. they comprise or consist of or essentially consist of inorganic or organic material.

Typically, they comprise or consist of or essentially consist of metal or an alloy of metals, or an organic material, or comprise or consist of or essentially consist of carbohydrate elements. Examples of material for microparticles include but are not limited to agarose, polystyrene, latex, polyvinyl alcohol, silica and ferromagnetic metals, alloys or composition materials. Microparticles may also comprise or consist of magnetic or ferromagnetic metals, alloys or compositions. The material may have specific properties such as e.g. being hydrophobic, or hydrophilic. Typically, microparticles are dispersed in aqueous solutions and retain a small negative surface charge keeping the microparticles separated and avoiding non-specific clustering. Magnetic or paramagnetic microparticles may be separated by magnetic forces. Magnetic forces are applied to pull the paramagnetic or magnetic particles out of the solution/suspension and to retain them as desired while liquid of the solution/suspension can be removed and the particles can e.g. be washed.

The term “buffer” or “buffer solution” refers to an aqueous solution comprising a mixture of a weak acid and its conjugate base, or vice versa. Its pH changes very little when a small or moderate amount of strong acid or base is added to it and thus it is used to prevent changes in the pH of a solution. Buffer solutions are used as a means of keeping pH at a nearly constant value in a wide variety of chemical applications. Common buffer compounds used include but are not limited to TAPS, Bicine, Tris, Tricine, TAPSO, HEPES, TES, MOPS, PIPES, Cacodylate, and MES.

A “kit” is any manufacture (e.g. a package or container) comprising at least one reagent, e.g., a medicament for treatment of a disorder, or a probe for specifically detecting a biomarker gene or protein of the invention. The manufacture is preferably promoted, distributed, or sold as a unit for performing the methods of the present invention. Typically, a kit may further comprise carrier means being compartmentalized to receive in close confinement one or more container means such as vials, tubes, and the like In particular, each of the container means comprises one of the separate elements to be used in the method of the first aspect. Kits may further comprise one or more other containers comprising further materials including but not limited to buffers, diluents, filters, needles, syringes, and package inserts with instructions for use. A label may be present on the container to indicate that the composition is used for a specific application, and may also indicate directions for either in vivo or in vitro use.

A “package insert” is used to refer to instructions customarily included in commercial packages of therapeutic products or medicaments, that contain information about the indications, usage, dosage, administration, contraindications, other therapeutic products to be combined with the packaged product, and/or warnings concerning the use of such therapeutic products or medicaments, etc.

### Embodiments

In a first aspect, the present invention relates to a method for measuring the level of VEGF-A in the presence of a VEGF-A antagonist. Said method comprises in a first step the incubation of a sample with a first and a second antibody, or with a fragment of a first and second antibody. Both, the first and the second antibody are capable of binding to VEGF-A or a variant thereof, in the presence of the VEGF-A antagonist. In particular, the first and the second antibody bind to VEGF-A in the presence of the VEGF-A antagonist.

In embodiments, the binding of the first and the binding of the second antibody does not interfere with each other. In particular embodiments, one of the first or the second antibody is capable of binding to a solid phase. In particular embodiments, the other of said first or second antibodies is detectably labeled. Upon incubation of said sample with said first and said second antibody, a detectably labeled complex is formed which comprises the first antibody, VEGF-A or the variant thereof, and the second antibody.

In a second step of the method for measuring the level of VEGF-A in the presence of a VEGF-A antagonist, the complex formed in the first step is detected. This detection allows for the measurement of the level of VEGF-A in the presence of a VEGF-A antagonist.

Accordingly, a first aspect relates to a method for measuring the level of VEGF-A in the presence of a VEGF-A antagonist, the method comprising: incubating a sample with a first and a second antibody, wherein said first and said second antibody are both capable of binding to VEGF-A in the presence of the VEGF-A antagonist and wherein the binding of said first and of said second antibody does not interfere with each other, wherein one of said antibodies is bound to or capable of binding to a solid phase and wherein the other of said antibodies is detectably labeled, thereby forming a detectably labeled complex comprising the first antibody, VEGF-A, and the second antibody, and detecting the complex formed, thereby measuring the level of VEGF-A in the presence of a VEGF-A antagonist.

In particular embodiments, the VEGF-A is human VEGF-A or a variant thereof. In particular embodiments, the VEGF-A comprises an amino acid sequence according to SEQ ID NO: 1 or a variant thereof. In particular embodiments, the VEGF-A consists of an amino acid sequence according to SEQ ID NO: 1 or a variant thereof. In particular embodiments, the variant of VEGF-A has the same functionality as VEGF-A, i.e. the variant is a functional variant. In particular embodiments, the variant of VEGF-A exhibits at least 80% sequence identity with the amino acid sequence of human VEGF-A, in particular according to SEQ ID NO: 1. In particular embodiments, the variant of VEGF-A exhibits at least 85%, 90%, 95%, 98% or 99% sequence identity with the amino acid sequence of human VEGF-A, in particular according to SEQ ID NO: 1. In particular embodiments, the variant of VEGF-A exhibits at least 85% or at least 95% sequence identity with the amino acid sequence of human VEGF-A, in particular according to SEQ ID NO: 1. In particular embodiments, the variant of VEGF-A exhibits 85%, 95%, or 98% sequence identity with the amino acid sequence of human VEGF-A, in particular according to SEQ ID NO: 1. In embodiments of the present invention, VEGF-A is present as monomer or as dimer, in particular as homodimer.

In particular embodiments, the VEGF-A is a human VEGF-A isoform or a variant thereof In particular embodiments, the VEGF-A isoform is the human VEGF-A isoform VEGF₁₂₁, VEGF₁₄₅, VEGF₁₆₅, VEGF₁₈₉ and/or VEGF₂₀₆, or a variant thereof In particular embodiments, the variant of the VEGF-A isoform has the same functionality as the respective VEGF-A isoform, i.e. the isoform variant is a functional isoform variant. In particular embodiments, the variant of the VEGF-A isoform exhibits at least 80% sequence identity with the amino acid sequence of the respective human VEGF-A isoform. In particular embodiments, the variant of a VEGF-A isoform exhibits at least 80% sequence identity with the amino acid sequence of human VEGF-A isoform. In particular embodiments, the variant of an VEGF-A isoform exhibits at least 85%, 90%, 95%, 98% or 99% sequence identity with the amino acid sequence of the respective human VEGF-A isoform. In particular embodiments, the variant of a VEGF-A isoform exhibits at least 85% or at least 95% sequence identity with the amino acid sequence of the respective human VEGF-A isoform. In particular embodiments, the variant of a VEGF-A isoform exhibits 85%, 95%, or 98% sequence identity with the amino acid sequence of the respective human VEGF-A isoform. Thus, in particular embodiments, the variant of the VEGF₁₂₁ isoform of VEGF-A exhibits 85%, 95%, or 98% sequence identity with the amino acid sequence of the human VEGF₁₂₁ isoform; the variant of the VEGF₁₄₅ isoform of VEGF-A exhibits 85%, 95%, or 98% sequence identity with the amino acid sequence of the human VEGF₁₄₅ isoform; the variant of the VEGF₁₆₅ isoform of VEGF-A exhibits 85%, 95%, or 98% sequence identity with the amino acid sequence of the human VEGF₁₆₅ isoform, the variant of the VEGF₁₈₉ isoform of VEGF-A exhibits 85%, 95%, or 98% sequence identity with the amino acid sequence of the human VEGF₁₈₉ isoform, and the variant of the VEGF₂₀₆ isoform of VEGF-A exhibits 85%, 95%, or 98% sequence identity with the amino acid sequence of the human VEGF₂₀₆ isoform.

In particular embodiments, the VEGF-A is a human VEGF-A fragment or a variant thereof In particular embodiments, the VEGF-A fragment is the human VEGF-A 110-amino acid fragment or a variant thereof In particular embodiments, the variant of the VEGF-A fragment has the same functionality as the respective VEGF-A fragment, i.e. the fragment variant is a functional fragment variant. In particular embodiments, the variant of the VEGF-A fragment exhibits at least 80% sequence identity with the amino acid sequence of the respective human VEGF-A isoform. In particular embodiments, the variant of a VEGF-A fragment exhibits at least 80% sequence identity with the amino acid sequence of human VEGF-A fragment. In particular embodiments, the variant of an VEGF-A fragment exhibits at least 85%, 90%, 95%, 98% or 99% sequence identity with the amino acid sequence of the respective human VEGF-A fragment. In particular embodiments, the variant of a VEGF-A fragment exhibits at least 85% or at least 95% sequence identity with the amino acid sequence of the respective human VEGF-A fragment. In particular embodiments, the variant of a VEGF-A fragment exhibits 85%, 95%, or 98% sequence identity with the amino acid sequence of the respective human VEGF-A fragment. Thus, in particular embodiments, the variant of the 110-amino acid fragment of VEGF -A exhibits 85%, 95%, or 98% sequence identity with the amino acid sequence of the human 110-amino acid VEGF-A fragment.

In embodiments, the VEGF-A antagonist prevents the interaction between VEGF-A and one or more of the VEGF receptor(s). In particular, the VEGF-A antagonist competes with VEGF-A at binding sites of the receptor or alters the binding site of VEGF-A for its receptor in a manner that it is not able to bind its receptor anymore, or is not able to trigger the functional action anymore which is normally cause by its binding. Accordingly, the VEGF-A antagonist may either bind to an epitope of VEGF-A and thereby hinder the binding of VEGF-A to its receptor, or the VEGF-A antagonist may bind to an epitope of the receptor and thereby prevent the binding of VEGF-A to the receptor. In particular embodiments, the VEGF-A antagonist binds to an epitope on VEGF-A and thereby prevents its binding to VEGF receptors. In particular embodiments, the VEGF-A receptor(s) is/are VEGFA-R1 and/or VEGFA-R2.

In further embodiments of the first aspect of the present invention, the VEGF-A antagonist is selected from the group consisting of a polypeptide, a protein, a peptibody, an immunoadhesin, a small molecule and an aptamer.

In particular embodiments, wherein the antagonist is a protein, said protein is an antibody. In a particular embodiment, the antibody is an anti-VEGF-A antibody. In particular, the anti-VEGF antibody is an antibody which binds to VEGF-A with sufficient affinity and specificity. In embodiments, the antibody has a sufficient binding affinity for VEGF-A. In particular, the antibody, or an antigen-binding fragment thereof, binds hVEGF-A with a K_{d} value of between 100 nM-1 pM, i.e. with a Kd value of 100 nM, 50 nM, 1 nM, 900 pM, 800 pM, 700 pm, 600 pM, 500 pM, 400 pM, 300 pM, 200 pM, 100 pM, 50 pM, or 1 pM. In particular embodiments, the antibody, or the antigen-binding fragment thereof, binds human VEGF-A (hVEGF-A) with a K_{d} value of between 50 nM-50 pM, 1 nM-100 pM, or 700 pM-300 pM.

In particular embodiments, the antagonistic VEGF-A antibody is monoclonal or polyclonal. In particular embodiments, the antagonistic antibody for VEGF-A is recombinantly produced. In further embodiments, the antagonistic VEGF-A antibody is a chimeric antibody, in particular a humanized anti-VEGF-A antibody. In particular embodiments, the antagonistic VEGF-A antibody comprises a mutated human IgG1 framework regions. The antagonistic VEGF-A antibody further comprises an antigen-binding complementarity-determining regions (CDR) from the murine anti-hVEGF monoclonal antibody A.4.6.1 that blocks binding of human VEGF to its receptors. In particular embodiments, 93% of the amino acid sequence of the antagonistic VEGF-A antibody, including most of the framework region, are derived from human IgG1, and about 7% of the sequence is derived from the murine antibody A4.6.1 (AVASTIN®). In particular embodiments, the antagonistic VEGF-A antibody is glycosylated. In further embodiments, the antagonistic VEGF-A antibody has a molecular mass of about 149,000 daltons. In particular embodiments, the antagonistic VEGF-A antibody is Bevacizumab (BV), also known as “rhuMAb VEGF” or “AVASTIN®”, which is a recombinant humanized anti-VEGF monoclonal antibody generated according to Presta et al. (1997) Cancer Res. 57:4593-4599.

In particular embodiments, the antagonistic VEGF-A antibody is an antibody fragment. The antibody fragment is selected from the group consisting of Fab-fragment, Fab′-fragment, F(ab′)2-fragment, single domain antibodies (sdAb), nanobodies, single chain Fv (scFv), divalent single-chain variable fragments (di-scFvs), tandem scFvs, diabodies, bispecific diabodies, single chain diabodies (scDb), Bi-specific T-cell engagers (BiTEs), and DART molecules. In particular embodiments, the antagonistic antibody fragment is a Fab-fragment or a F(ab′)₂-fragment, in particular a humanized Fab fragment or a humanized or a F(ab′)₂-fragment.

In further embodiments, the VEGF-A antagonist is selected from the group consisting of VEGF-Trap, Mucagen, PTK787, SU11248, AG-013736, Bay 439006 (sorafenib), ZD-6474, CP632, CP-547632, AZD-2171, CDP-171, SU-14813, CHIR-258, AEE-788, SB786034, BAY579352, CDP-791, EG-3306, GW-786034, RWJ-417975/CT6758 and KRN-633.

In particular embodiments of the first aspect, a first antibody against VEGF-A and a second antibody against VEGF-A, are used, wherein said first antibody and said second antibody both bind to VEGF-A at identical or at different epitopes. In particular embodiments, said first antibody and said second antibody both bind to VEGF-A at different epitopes.

In particular embodiments, the first and the second antibody do not interfere with each other. Accordingly, the binding of one of these antibodies does not prevent or diminish the binding of the respective other antibody. In embodiments of the present invention, the first and second antibody bind to two different epitopes on the same monomer and/or to two different epitopes on each monomer of a dimer. Alternatively, the first and the second antibody bind to the same, or substantially the same epitope, on different monomers of a homodimer. In particular embodiments, said first antibody and said second antibody both bind to VEGF-A at different epitopes.

In particular embodiments, the first antibody and the second antibody individually of each other, bind to an epitope which is covered by or bound by a VEGF receptor, in particular by the VEGF-A receptor VEGFA-R1 and/or VEGFA-R2. Thus, said first antibody and said second antibodies, individually of each other, bind to the identical epitope as a VEGF-A receptor, in particular as VEGFA-R1 or VEGFA-R2. Alternatively, said first antibody and said second antibodies, individually of each other, bind to an epitope which is not directly bound by the VEGF-A receptor, such as e.g. VEGFA-R1 or VEGFA-R2, but which is covered by the receptors such that the binding of the first and/or second antibody prevents the binding of the VEGF-A receptor(s). Accordingly, in particular embodiments, the first antibody and/or the second antibody compete for the binding of VEGFA-R1 and/or VEGFA-R2.

In particular embodiments, the first and the second antibody individually of each other, bind to the same or to a different epitope as the VEGF-A antagonist, in particular to a different epitope as the antagonistic antibody. In case the first or the second antibody binds to the same epitope as the antagonist, in particular the antagonistic antibody, it is envisaged that the first or second antibody binds the epitope with a lower Kd value than the antagonist. In particular embodiments, the first or second antibody binds the epitope with a Kd value of below 1.5 nM, in particular below 1 nM, below 0.75 nM, in particular below 0.5 nM.

In embodiments of the first aspect, either the first antibody or the second antibody binds to the epitope as bound by an antibody comprising CDRs selected from the group consisting of amino acids 46-51 of SEQ ID NO: 2, amino acids 69-71 of SEQ ID NO: 2, amino acids 108-116 of SEQ ID NO: 2, amino acids 44-52 of SEQ ID NO: 3, amino acids 70-76 of SEQ ID NO: 3, amino acids 115-125 of SEQ ID NO: 3, amino acids 47-52 of SEQ ID NO: 4, amino acids 70-72 of SEQ ID NO: 4, amino acids 109-117 of SEQ ID NO: 4, amino acids 45-52 of SEQ ID NO: 5, amino acids 70-77 of SEQ ID NO: 5, and amino acids 116-126 of SEQ ID NO: 5.

In embodiments of the first aspect, the first antibody and/or the second antibody binds to the epitope as bound by an antibody comprising CDRs selected from the group consisting of amino acids 46-51 of SEQ ID NO: 2, amino acids 69-71 of SEQ ID NO: 2, amino acids 108-116 of SEQ ID NO: 2, amino acids 44-52 of SEQ ID NO: 3, amino acids 70-76 of SEQ ID NO: 3, and amino acids 115-125 of SEQ ID NO: 3. In particular embodiments, the first antibody and/or the second antibody binds to the epitope as bound by an antibody comprising the CDRs consisting of amino acids 46-51 of SEQ ID NO: 2, amino acids 69-71 of SEQ ID NO: 2, amino acids 108-116 of SEQ ID NO: 2, amino acids 44-52 of SEQ ID NO: 3, amino acids 70-76 of SEQ ID NO: 3, and amino acids 115-125 of SEQ ID NO: 3.

In embodiments of the first aspect, the first antibody and/or the second antibody binds to the epitope as bound by an antibody comprising CDRs selected from the group consisting of amino acids 47-52 of SEQ ID NO: 4, amino acids 70-72 of SEQ ID NO: 4, amino acids 109-117 of SEQ ID NO: 4, amino acids 45-52 of SEQ ID NO: 5, amino acids 70-77 of SEQ ID NO: 5, and amino acids 116-126 of SEQ ID NO: 5. In particular embodiments, the first antibody and/or the second antibody binds to the epitope as bound by an antibody comprising CDRs consisting of amino acids 47-52 of SEQ ID NO: 4, amino acids 70-72 of SEQ ID NO: 4, amino acids 109-117 of SEQ ID NO: 4, amino acids 45-52 of SEQ ID NO: 5, amino acids 70-77 of SEQ ID NO: 5, and amino acids 116-126 of SEQ ID NO: 5.

In embodiments of the first aspect, the first antibody and/or the second antibody binds to the epitope as bound by an antibody comprising FR selected from the group consisting of amino acids 20-45 of SEQ ID NO: 2, amino acids 52-68 of SEQ ID NO: 2, amino acids 72-107 of SEQ ID NO: 2, amino acid 117-126 of SEQ ID NO: 2, amino acids 19-43 of SEQ ID NO: 3, amino acids 53-69 of SEQ ID NO: 3, and amino acids 77-114 of SEQ ID NO: 3, amino acids 126-136 of SEQ ID NO: 3. In particular embodiments, the first antibody and/or the second antibody binds to the epitope as bound by an antibody comprising the FRs consisting of amino acids 20-45 of SEQ ID NO: 2, amino acids 51-68 of SEQ ID NO: 2, amino acids 72-107 of SEQ ID NO: 2, amino acid 117-126 of SEQ ID NO: 2, amino acids 19-43 of SEQ ID NO: 3, amino acids 51-69 of SEQ ID NO: 3, and amino acids 77-114 of SEQ ID NO: 3, amino acids 126-136 of SEQ ID NO: 3.

In embodiments of the first aspect, the first antibody and/or the second antibody binds to the epitope as bound by an antibody comprising FRs selected from the group consisting of amino acids 21-46 of SEQ ID NO: 4, amino acids 53-69 of SEQ ID NO: 4, amino acids 73-108 of SEQ ID NO: 4, amino acids 118-127 of SEQ ID NO: 4, amino acids 20-44 of SEQ ID NO: 5, amino acids 53-69 of SEQ ID NO: 5, and amino acids 78-115 of SEQ ID NO: 5, amino acids 127-137 of SEQ ID NO: 5. In particular embodiments, the first antibody and/or the second antibody binds to the epitope as bound by an antibody comprising FRs consisting of amino acids 21-46 of SEQ ID NO: 4, amino acids 53-69 of SEQ ID NO: 4, amino acids 73-108 of SEQ ID NO: 4, amino acids 118-127 of SEQ ID NO: 4, amino acids 20-44 of SEQ ID NO: 5, amino acids 53-69 of SEQ ID NO: 5, and amino acids 78-115 of SEQ ID NO: 5, amino acids 127-137 of SEQ ID NO: 5.

In particular embodiments, the first antibody and/or the second antibody binds to the epitope as bound by an antibody comprising CDRs consisting of amino acids 46-51 of SEQ ID NO: 2, amino acids 69-71 of SEQ ID NO: 2, amino acids 108-116 of SEQ ID NO: 2, amino acids 44-52 of SEQ ID NO: 3, amino acids 70-76 of SEQ ID NO: 3, and amino acids 115-125 of SEQ ID NO: 3; and comprising FRs consisting of amino acids 20-45 of SEQ ID NO: 2, amino acids 52-68 of SEQ ID NO: 2, amino acids 72-107 of SEQ ID NO: 2, amino acid 117-126 of SEQ ID NO: 2, amino acids 19-43 of SEQ ID NO: 3, amino acids 53-69 of SEQ ID NO: 3, and amino acids 77-114 of SEQ ID NO: 3, amino acids 126-136 of SEQ ID NO: 3.

In particular embodiments, the first antibody and/or the second antibody binds to the epitope as bound by an antibody comprising CDRs consisting of amino acids 47-52 of SEQ ID NO: 4, amino acids 70-72 of SEQ ID NO: 4, amino acids 109-117 of SEQ ID NO: 4, amino acids 45-52 of SEQ ID NO: 5, amino acids 70-77 of SEQ ID NO: 5, and amino acids 116-126 of SEQ ID NO: 5, and comprising FRs consisting of amino acids 21-46 of SEQ ID NO: 4, amino acids 53-69 of SEQ ID NO: 4, amino acids 73-108 of SEQ ID NO: 4, amino acids 118-127 of SEQ ID NO: 4, amino acids 20-44 of SEQ ID NO: 5, amino acids 53-69 of SEQ ID NO: 5, and amino acids 78-115 of SEQ ID NO: 5, amino acids 127-137 of SEQ ID NO: 5.

In embodiments of the first aspect, the first and/or the second antibody comprises an amino acid sequences selected from the group consisting of SEQ ID NO: 2, 3, 4 and 5.

In embodiments of the first aspect, the first and/or the second antibody comprises a light chain having an amino acid sequences selected from the group consisting of SEQ ID NO: 2 and 4.

In embodiments of the first aspect, the first and/or the second antibody comprises a heavy chain having an amino acid sequences selected from the group consisting of SEQ ID NO: 3 and 5.

In embodiments of the first aspect, the first or the second antibody comprises a light chain having an amino acid sequence of SEQ ID NO: 2 and a heavy chain having an amino acid sequences of SEQ ID NO: 3.

In embodiments of the first aspect, the first or the second antibody comprises a light chain having an amino acid sequence of SEQ ID NO: 4 and a heavy chain having an amino acid sequences of SEQ ID NO: 5.

In embodiments of the first aspect, one of the first antibody or the second antibody is detectably labeled. Said label may be a molecule detectable by spectroscopic, photochemical, biochemical, immunochemical, chemical, or other physical means.

In particular embodiments, the first or the second antibody may be labeled with a fluorescent dye, electron-dense reagent, enzyme (e.g., as commonly used in an ELISA), biotin, digoxigenin, or hapten and other entities which are or can be made detectable. In particular embodiments, the first or second antibody is biotinylated or ruthenylated. Methods for labeling of an antibody are well-known to the person skilled in the art and abundantly described e.g. in Haugland (2003) Molecular Probes Handbook of Fluorescent Probes and Research Chemicals, Molecular Probes, Inc.; Brinkley (1992) Bioconjugate Chem. 3:2; Garman, (1997) Non-Radioactive Labeling: A Practical Approach, Academic Press, London; Means (1990) Bioconjugate Chem. 1:2; Glazer et al Chemical Modification of Proteins. Laboratory Techniques in Biochemistry and Molecular Biology (T. S. Work and E. Work, Eds.) American Elsevier Publishing Co., New York; Lundblad, R. L. and Noyes, C. M. (1984) Chemical Reagents for Protein Modification, Vols. I and II, CRC Press, New York; Pfleiderer, G. (1985) “Chemical Modification of Proteins”, Modern Methods in Protein Chemistry, H. Tschesche, Ed., Walter DeGruyter, Berlin and New York; and Wong (1991) Chemistry of Protein Conjugation and Cross-linking, CRC Press, Boca Raton, Fla.); DeLeon-Rodriguez et al, Chem. Eur. J. 10 (2004) 1149-1155; Lewis et al, Bioconjugate Chem. 12 (2001) 320-324; Li et al, Bioconjugate Chem. 13 (2002) 110-115; Mier et al Bioconjugate Chem. 16 (2005) 240-237.

In particular embodiments, one of the first antibody or the second antibody is capable of binding to a solid phase or is bound to a solid phase.

In particular embodiments, the first antibody is capable of binding to a solid phase or is bound to a solid phase, and the second antibody or antigen-binding fragment thereof, is detectably labeled.

In further embodiments of the first aspect, the first antibody is bound to a solid phase or is capable of binding to a solid phase and has a light chain comprising or consisting of a sequence according to SEQ ID NO: 2, and the second antibody is detectably labeled and has a light chain comprising or consisting of a sequence according to SEQ ID NO: 4.

In further embodiments of the first aspect, the first antibody is bound to a solid phase or is capable of binding to a solid phase and has a light chain comprising or consisting of a sequence according to SEQ ID NO: 4, and the second antibody is detectably labeled and has a light chain comprising or consisting of a sequence according to SEQ ID NO: 2.

In further embodiments of the first aspect, the first antibody is bound to a solid phase or is capable of binding to a solid phase and has a heavy chain comprising or consisting of a sequence according to SEQ ID NO: 3, and the second antibody is detectably labeled and has a heavy chain comprising or consisting of a sequence according to SEQ ID NO: 5.

In further embodiments of the first aspect, the first antibody is bound to a solid phase or is capable of binding to a solid phase and has a heavy chain comprising or consisting of a sequence according to SEQ ID NO: 5, and the second antibody is detectably labeled and has a heavy chain comprising or consisting of a sequence according to SEQ ID NO: 3.

In further embodiments of the first aspect, the first antibody is bound to a solid phase or is capable of binding to a solid phase and has a light chain comprising or consisting of a sequence according to SEQ ID NO: 2 and a heavy chain comprising or consisting of a sequence according to SEQ ID NO: 3, and the second antibody is detectably labeled and has a light chain comprising or consisting of a sequence according to SEQ ID NO: 4 and a heavy chain comprising or consisting of a sequence according to SEQ ID NO: 5.

In further embodiments of the first aspect, the first antibody is bound to a solid phase or is capable of binding to a solid phase and has a light chain comprising or consisting of a sequence according to SEQ ID NO: 4 and a heavy chain comprising or consisting of a sequence according to SEQ ID NO: 5, and the second antibody is detectably labeled and has a light chain comprising or consisting of a sequence according to SEQ ID NO: 2 and a heavy chain comprising or consisting of a sequence according to SEQ ID NO: 3.

In further embodiments, the solid phase is in a form selected from the group consisting of beads, tubes, discs of microplates, and any other surface suitable, in particular suitable for conducting an immunoassay. In particular embodiments, the beads are microbeads. Microbeads are microparticles with a diameter in the nanometer and micrometer range. In embodiments, the microparticles may have a diameter of 50 nanometers to 50 micrometers. In particular, the microparticles have a diameter of between 100 nm and 10 μm, in particular of 200 nm to 5 μm, or of 750 nm to 5 μm. Microparticles comprise or consist of any suitable material known to the person skilled in the art, e.g. they comprise or consist of or essentially consist of inorganic or organic material. In particular, they comprise or consist of or essentially consist of metal or an alloy of metals, or an organic material, or comprise or consist of or essentially consist of carbohydrate elements. In particular embodiments, the material of the microparticles is selected from the group consisting of agarose, polystyrene, latex, polyvinyl alcohol, silica and ferromagnetic metals, alloys or composition materials. Microparticles may also comprise or consist of magnetic or ferromagnetic metals, alloys or compositions. The material may have specific properties such as e.g. being hydrophobic, or hydrophilic. In particular embodiments, the microparticles are dispersed in aqueous solutions and retain a small negative surface charge keeping the microparticles separated and avoiding non-specific clustering.

In particular embodiments, the magnetic or paramagnetic microparticles are separated by magnetic forces. Magnetic forces are applied to pull the paramagnetic or magnetic particles out of the solution/suspension and to retain them as desired while liquid of the solution/suspension can be removed and the particles can e.g. be washed.

In particular embodiments, the first or the second antibody is an IgG antibody. In particular embodiments, the first or the second antibody is an IgG2 antibody. In particular embodiments, the first or the second antibody is an IgG2b antibody, or an antigen-binding fragment thereof, in particular an IgG2b-F(ab′)₂ fragment.

In embodiments of the first aspect, the sample is derived from or is body fluid, in particular selected from the group consisting of whole blood, blood serum, blood plasma, urine, saliva and sputum. In particular embodiments, the sample is derived from or is a whole blood sample, blood serum, or blood plasma.

In embodiments, the sample is derived from a healthy individual or from a patient. In particular embodiments, the patient suffers from a proliferative disorder, in particular from cancer, in particular from metastatic cancers. In particular embodiments, the patient suffers from cancer, in particular from metastatic cancers, and is treated with a VEGF-A antagonist. In particular embodiments, the patient suffers from cancer, in particular from metastatic cancers, and is treated with Bevacizumab.

In particular embodiments, the cancer is selected from the group consisting of carcinoma, lymphoma, blastoma, sarcoma, and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including, e.g., gastrointestinal cancer), pancreatic cancer (including, e.g., metastic pancreatic cancer), glioblastoma, cervical cancer, ovarian cancer, liver cancer, bladder cancer, hepatoma, breast cancer (including locally advanced, recurrent or metastatic HER-2 negative breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. In particular, the patient suffers from cancer selected from the group consisting of colon cancer, lung cancers, renal cancers, ovarian cancers, and glioblastoma multiforme of the brain.

In particular embodiments, the patient is a mammal, reptile, bird or fish. In particular embodiments, the patient is mammal selected from the group consisting of mouse, rat, rabbit, or zebrafish guinea pig, rabbit, horse, donkey, cow, sheep, goat, pig, chicken, camel, cat, dog, turtle, tortoise, snake, lizard, goldfish and primates. In particular, the patient is a human being.

In further embodiments of the first aspect, the method for measuring the level of VEGF-A in the presence of a VEGF-A antagonist is an immunoassay, in particular a sandwich immuno-assay, wherein an antibody—antigen—antibody complex, also called a sandwich, is formed. The skilled artisan will appreciate that in a sandwich assay for the detection of VEGF-A, the first antibody may act as a capture antibody and the second antibody may act as a tracer antibody. Alternatively, the second antibody may act as a capture antibody and the first antibody may act as a tracer antibody.

In particular embodiments of measuring the level of VEGF-A in a sample, the first and second antibody are mixed with the sample to be analyzed.

In one embodiment, wherein a sandwich assay is performed without washing step, such mixing/incubation is performed in a single reaction vessel. The sequence of adding and mixing the three ingredients (e.g. microparticles coated with first antibody, or antigen-binding fragment thereof, sample, second detectably-labeled antibody, or antigen-binding fragment thereof, respectively) is not critical. This mixture is incubated for a period of time sufficient for the first antibody (in particular the first antibody coated onto the microparticles) and the detectably labeled second antibody, to bind to VEGF-A.

In another embodiment, wherein a sandwich assay is performed with a washing step, the adding and mixing of the first antibody (in particular the first antibody coated onto microparticles), sample and detectably-labeled second antibody, or antigen-binding fragment(s) thereof, is performed sequentially into a single reaction vessel. In a first step (the analyte-capturing step) the microparticles coated with the first antibody are incubated with the sample to be analyzed for a period of time sufficient for the analyte, i.e. VEGF-A, to be bound. Following a washing step, the detectably-labeled second antibody is added and incubated for a period of time sufficient for the second antibody to bind to the analyte, i.e. VEGF-A. In embodiments, the method of the first aspect is practiced in a competitive assay format.

In embodiments, the mixture in incubated for less than 60 min, i.e. less than 60, 55, 50, 45, 40, 35, 30, 25, 20, 15, 10, or 5 min. In particular embodiments, the mixture is incubated for 4 min to 1 hour (i.e. 4, 5, 6, 7, 8, 9, 10, 15, 20, 25, 30, 35, 40, 45, 50, 55, or 60 min). In particular embodiments, the mixture is incubated for 5 min to 45 min, i.e. for 5, 6, 7, 8, 9, 10, 15, 20, 25, 30, 35, 40, or 45 min). In particular embodiments, the mixture is incubated for 5 min to 30 min, i.e. for 5, 6, 7, 8, 9, 10, 15, 20, 25, or 30 min. In particular embodiments, the mixture is incubated for 9 or 18 min. In embodiments, the mixture is incubated for 1-12 hours (i.e. 1, 2, 3, 4, 5, 6, 7, 8, 9, 10, 11, or 12 hours). In particular embodiments, the mixture is incubated for 1-4 hours or for 8-12 hours.

In further embodiments, the mixture is incubated at a temperature of 3-40° C. (i.e. 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40° C.). In particular, the mixture is incubated for 3° C. to 8° C. (i.e. 3, 4, 5, 6, 7 or 8), in particular at 4-5° C., or at 20° C. to 25° C. (i.e. at 20, 21, 22, 23, 24, or 25° C.), in particular 20-22° C., or at 35-37° C.

It is well-known to the person skilled in the art that incubation temperature and incubation time depend upon each other. Accordingly, in particular embodiments, the mixture is incubated at 20-25° C. for 10 min to 1 hours, i.e. the mixture is incubated at 20, 21, 22, 23, 24, or 25° C. for 10, 15, 20, 25, 30, 35, 40, 45, 50, 55, or 60 min. In particular embodiments, the mixture is incubated for less than 10 min or less than 20 min at 22° C. In embodiments, the mixture is incubated for 1-12 hours at 3-8° C. In particular, the mixture is incubated for 1-4 hours or for 8-12 hours at 3-8° C., in particular at 4-5° C.

The first and/or the second antibody are incubated for a period of time sufficient for the first antibody coated onto the microparticles and the detectably labeled second antibody, to bind to VEGF-A in the sample.

In particular embodiments, the first and/or the second antibody is/are comprised in and/or are incubated in a physiological solution, in particular in a physiological buffer. In particular embodiments, the buffer is selected from the group of TAPS, Bicine, Tris, Tricine, TAPSO, HEPES, TES, MOPS, PIPES, Cacodylate, and MES. In particular embodiments, the buffer is an MES buffer. In particular embodiments, the IVIES buffer comprises the following components: 50 mM MES, 150 mM NaCl, 2 mM EDTA-Na₂ (dihydrate), 0.1% N-Methylisothiazolon-HCl, 0.1% Oxypyrion, 0.1% Polydocanol (Thesit), 1.0% Albumin RPLA 4 assay quality, 0.2% PAK<->R-IgG(DET), Millipore-water, pH adjusted to 6.30 with 2N NaOH.

In particular embodiments, the formed antibody—antigen—antibody complex, in particular the complex formed comprising the first antibody, VEGF-A—the second antibody, is detected via any method well-known in the art. In particular embodiments, the complex formed is detected via electrochemiluminescent, chemiluminescent, or fluorescence.

In a second aspect, the present invention relates to a kit for measuring the level of VEGF-A in the presence of a VEGF-A antagonist. Said kit comprises means for detecting VEGF-A in the presence of a VEGF-A antagonist.

In particular embodiments, said kit comprises a first and a second antibody. The first and the second antibody are capable of binding to VEGF-A in the presence of the VEGF-A antagonist. In particular, the binding of said first and of said second antibody does not interfere with each other. Further, one of said antibodies is bound to or capable of binding to a solid phase and the other of said antibodies is detectably labeled. Accordingly, in a second aspect, the present invention relates to a kit for measuring the level of VEGF-A in the presence of a VEGF-A antagonist, the kit comprising: a first and a second antibody wherein said first and said second antibody are capable of binding to VEGF-A in the presence of the VEGF-A antagonist, and wherein the binding of said first and of said second antibody does not interfere with each other, wherein one of said antibodies is bound to or capable of binding to a solid phase and wherein the other of said antibodies is detectably labeled.

In embodiments, the kit further comprises carrier means being compartmentalized to receive in close confinement one or more container means selected from the group consisting of vials and tubes. In particular embodiment, the container means further comprise one of several separate elements to be used, in particular those selected from the group consisting of buffers, diluents, filters, needles, syringes, and package inserts with instructions for use. A label may be present on the container to indicate that the composition is used for a specific application, and may also indicate directions for either in vivo or in vitro use.

In particular embodiments, the kit comprises at least one container, a label on said at least one container, and a composition contained within said at least one container, wherein the composition includes at least one antibody that binds to VEGF-A.

In particular, the kit comprises at least one container comprising the first and the second antibody, or the kit comprises at least two containers wherein one container comprises the first antibody and the second container comprises the second antibody.

In particular, the label on said container indicates that the composition can be used to evaluate the presence of VEGF-A in a sample. In particular, the kit includes instructions for using the antibody for evaluating the presence of VEGF-A in a particular sample type. The kit may further comprise a set of instructions and materials for preparing a sample and applying antibody to the sample.

In embodiments, the kit further comprises a VEGF-A antagonist.

In particular embodiments, the kit comprises one container comprising the first antibody, the second antibody, and the VEGF-A antagonist, as specified above within the context of the first aspect or as specified below in the context of the further aspects.

In particular embodiments, the kit comprises two containers, wherein the first container comprises the first antibody and the second antibody, and wherein the second container comprises the VEGF-A antagonist, as specified above within the context of the first aspect or as specified below in the context of the further aspects.

In particular embodiments, the kit comprises three containers, wherein the first container comprises the first antibody, the second container comprises the second antibody, and the third container comprises the VEGF-A antagonist, as specified above within the context of the first aspect or as specified below in the context of the third aspect.

In further embodiments, the kits also comprise components selected from the group consisting of one or more buffers (e.g., block buffer, wash buffer, substrate buffer, etc.), other reagents such as substrate (e.g., chromogen) that is chemically altered by an enzymatic label, epitope retrieval solution, control samples (positive and/or negative controls), control slide(s), etc. Kits can also include instructions for interpreting the results obtained using the kit.

In particular embodiments, the first antibody, the second antibody, and/or the VEGF-A antagonist comprised in the one or more container are present in lyophilized form or in solubilized form. In particular embodiments, the first antibody, the second antibody, and/or the VEGF-A antagonist comprised in the one or more container are comprised in a solution, in particular in a physiological solution. In particular embodiments, the first antibody, the second antibody, and/or the VEGF-A antagonist comprised in the one or more container are comprised in a physiological buffer, in particular in a buffer selected from the group of TAPS, Bicine, Tris, Tricine, TAPSO, HEPES, TES, MOPS, PIPES, Cacodylate, and MES. In particular embodiments, the first antibody, the second antibody, and/or the VEGF-A antagonist comprised in the one or more container are comprised in IVIES buffer. In particular embodiments, the MES buffer comprises the following components: 50 mM MES, 150 mM NaCl, 2 mM EDTA-Na₂ (dihydrate), 0.1% N-Methylisothiazolon-HCl, 0.1% Oxypyrion, 0.1% Polydocanol (Thesit), 1.0% Albumin RPLA 4 assay quality, 0.2% PAK<->R-IgG(DET), Millipore-water, pH adjusted to 6.30 with 2N NaOH

In embodiments of the second aspect, the kit comprises the antibodies as specified above with regard to the first aspect and/or as specified below with regard to the further aspects.

In embodiments, the VEGF-A antagonist prevents the interaction between VEGF-A and one or more of the VEGF receptor(s). In particular, the VEGF-A antagonist competes with VEGF-A at binding sites of the receptor or alters the binding site of VEGF-A for its receptor in a manner that it is not able to bind its receptor anymore, or is not able to trigger the functional action anymore which is normally cause by its binding. Accordingly, the VEGF-A antagonist may either bind to an epitope of VEGF-A and thereby hinder the binding of VEGF-A to its receptor, or the VEGF-A antagonist may bind to an epitope of the receptor and thereby prevent the binding of VEGF-A to the receptor. In particular embodiments, the VEGF-A antagonist binds to an epitope on VEGF-A and thereby prevents its binding to VEGF receptors. In particular embodiments, the VEGF-A receptor(s) is/are VEGFA-R1 and/or VEGFA-R2.

In further embodiments of the second aspect of the present invention, the VEGF-A antagonist is selected from the group consisting of a polypeptide, a peptibody, an immunoadhesin, a small molecule and an aptamer.

In particular embodiments, wherein the antagonist is a polypeptide, said polypeptide is an antibody. In a particular embodiment, the antibody is an anti-VEGF-A antibody. In particular, the anti-VEGF antibody is an antibody which binds to VEGF-A with sufficient affinity and specificity. In embodiments, the antibody has a sufficient binding affinity for VEGF-A. In particular, the antibody binds hVEGF-A with a K_{d} value of between 100 nM-1 pM, i.e. with a Kd value of 100 nM, 50 nM, 1 nM, 900 pM, 800 pM, 700 pm, 600 pM, 500 pM, 400 pM, 300 pM, 200 pM, 100 pM, 50 pM, or 1 pM. In particular embodiments, the antibody binds human VEGF-A (hVEGF-A) with a K_{d} value of between 50 nM-50 pM, 1 nM-100 pM, or 700 pM-300 pM.

In particular embodiments, the antagonistic VEGF-A antibody is monoclonal or polyclonal. In particular embodiments, the antagonistic antibody for VEGF-A is recombinantly produced. In further embodiments, the antagonistic VEGF-A antibody, is a chimeric antibody in particular, a humanized anti-VEGF-A antibody. In particular embodiments, the antagonistic VEGF-A antibody comprises a mutated human IgG1 framework regions. The antagonistic VEGF-A antibody further comprises an antigen-binding complementarity-determining regions (CDR) from the murine anti-hVEGF monoclonal antibody A.4.6.1 that blocks binding of human VEGF to its receptors. In particular embodiments, 93% of the amino acid sequence of the antagonistic VEGF-A antibody, including most of the framework region, are derived from human IgG1, and about 7% of the sequence is derived from the murine antibody A4.6.1 (AVASTIN®). In particular embodiments, the antagonistic VEGF-A antibody is glycosylated. In further embodiments, the antagonistic VEGF-A antibody has a molecular mass of about 149,000 daltons. In particular embodiments, the antagonistic VEGF-A antibody is Bevacizumab (BV), also known as “rhuMAb VEGF” or “AVASTIN®”, which is a recombinant humanized anti-VEGF monoclonal antibody generated according to Presta et al. (1997) Cancer Res. 57:4593-4599.

In particular embodiments, the antagonistic VEGF-A antibody is an antigen-binding antibody fragment. The antibody fragment is selected from the group consisting of Fab-fragment, Fab'-fragment, F(ab′)2-fragment, single domain antibodies (sdAb), nanobodies, single chain Fv (scFv), divalent single-chain variable fragments (di-scFvs), tandem scFvs, diabodies, bispecific diabodies, single chain diabodies (scDb), Bi-specific T-cell engagers (BiTEs), and DART molecules. In particular embodiments, the antagonistic antibody fragment is a Fab-fragment or a F(ab′)2-fragment, in particular a humanized Fab fragment or a humanized or a F(ab′)2-fragment.

In further embodiments, the VEGF-A antagonist is selected from the group consisting of VEGF-Trap, Mucagen, PTK787, SU11248, AG-013736, Bay 439006 (sorafenib), ZD-6474, CP632, CP-547632, AZD-2171, CDP-171, SU-14813, CHIR-258, AEE-788, SB786034, BAY579352, CDP-791, EG-3306, GW-786034, RWJ-417975/CT6758 and KRN-633.

In particular embodiments of the second aspect, the kit comprises a first antibody against VEGF-A and a second antibody against VEGF-A wherein said first antibody and said second antibody both bind to VEGF-A at identical or at different epitopes. In particular embodiments, said first antibody and said second antibody both bind to VEGF-A at different epitopes.

VEGF-A is present as monomer or as dimer, in particular as homodimer. In particular embodiments, the first and the second antibody do not interfere with each other. Accordingly, the binding of one of these antibodies does not prevent or diminish the binding of the respective other antibody. In embodiments of the second aspect, the first and second antibody bind to two different epitopes on the same monomer and/or to two different epitopes on each monomer of the dimer. Alternatively, the first and the second antibody bind to the same, or substantially the same epitope, on different monomers of the homodimer. In particular embodiments, said first antibody and said second antibody both bind to VEGF-A at different epitopes.

In particular embodiments, the first and the second antibody individually of each other, bind to the same or to a different epitope as the VEGF-A antagonist, in particular to a different epitope as the antagonistic antibody. In case the first or the second antibody binds to the same epitope as the antagonist, in particular the antagonistic antibody, it is envisaged that the first or second antibody binds the epitope with a lower Kd value than the antagonist. In particular embodiments, the first or second antibody binds the epitope with a Kd value of below 1.5 nM, in particular below 1 nM, below 0.75 nM, in particular below 0.5 nM.

In particular embodiments, the first antibody and the second antibody individually of each other, bind to an epitope which is covered by or bound by a VEGF receptor, in particular by the VEGF-A receptor VEGFA-R1 and/or VEGFA-R2. Thus, said first antibody and said second antibodies, individually of each other, bind to the identical epitope as a VEGF-A receptor, in particular as VEGFA-R1 or VEGFA-R2. Alternatively, said first antibody and said second antibodies, individually of each other, bind to an epitope which is not directly bound by the VEGF-A receptor, such as e.g. VEGFA-R1 or VEGFA-R2, but which is covered by the receptors such that the binding of the first and/or second antibody prevents the binding of the VEGF-A receptor(s). Accordingly, in particular embodiments, the first antibody and/or the second antibody compete for the binding of VEGFA-R1 and/or VEGFA-R2.

In embodiments of the second aspect, either the first antibody or the second antibody binds to the epitope as bound by an antibody comprising CDRs selected from the group consisting of amino acids 46-51 of SEQ ID NO: 2, amino acids 69-71 of SEQ ID NO: 2, amino acids 108-116 of SEQ ID NO: 2, amino acids 44-52 of SEQ ID NO: 3, amino acids 70-76 of SEQ ID NO: 3, amino acids 115-125 of SEQ ID NO: 3, amino acids 47-52 of SEQ ID NO: 4, amino acids 70-72 of SEQ ID NO: 4, amino acids 109-117 of SEQ ID NO: 4, amino acids 45-52 of SEQ ID NO: 5, amino acids 70-77 of SEQ ID NO: 5, and amino acids 116-126 of SEQ ID NO: 5.

In embodiments of the second aspect, the first antibody and/or the second antibody binds to the epitope as bound by an antibody comprising CDRs selected from the group consisting of amino acids 46-51 of SEQ ID NO: 2, amino acids 69-71 of SEQ ID NO: 2, amino acids 108-116 of SEQ ID NO: 2, amino acids 44-52 of SEQ ID NO: 3, amino acids 70-76 of SEQ ID NO: 3, and amino acids 115-125 of SEQ ID NO: 3. In particular embodiments, the first antibody and/or the second antibody binds to the epitope as bound by an antibody comprising the CDRs consisting of amino acids 46-51 of SEQ ID NO: 2, amino acids 69-71 of SEQ ID NO: 2, amino acids 108-116 of SEQ ID NO: 2, amino acids 44-52 of SEQ ID NO: 3, amino acids 70-76 of SEQ ID NO: 3, and amino acids 115-125 of SEQ ID NO: 3.

In embodiments of the second aspect, the first antibody and/or the second antibody binds to the epitope as bound by an antibody comprising CDRs selected from the group consisting of amino acids 47-52 of SEQ ID NO: 4, amino acids 70-72 of SEQ ID NO: 4, amino acids 109-117 of SEQ ID NO: 4, amino acids 45-52 of SEQ ID NO: 5, amino acids 70-77 of SEQ ID NO: 5, and amino acids 116-126 of SEQ ID NO: 5. In particular embodiments, the first antibody and/or the second antibody binds to the epitope as bound by an antibody comprising CDRs consisting of amino acids 47-52 of SEQ ID NO: 4, amino acids 70-72 of SEQ ID NO: 4, amino acids 109-117 of SEQ ID NO: 4, amino acids 45-52 of SEQ ID NO: 5, amino acids 70-77 of SEQ ID NO: 5, and amino acids 116-126 of SEQ ID NO: 5.

In embodiments of the second aspect, the first antibody and/or the second antibody binds to the epitope as bound by an antibody comprising FR selected from the group consisting of amino acids 20-45 of SEQ ID NO: 2, amino acids 52-68 of SEQ ID NO: 2, amino acids 72-107 of SEQ ID NO: 2, amino acid 117-126 of SEQ ID NO: 2, amino acids 19-43 of SEQ ID NO: 3, amino acids 53-69 of SEQ ID NO: 3, and amino acids 77-114 of SEQ ID NO: 3, amino acids 126-136 of SEQ ID NO: 3. In particular embodiments, the first antibody and/or the second antibody binds to the epitope as bound by an antibody comprising the FRs consisting of amino acids 20-45 of SEQ ID NO: 2, amino acids 51-68 of SEQ ID NO: 2, amino acids 72-107 of SEQ ID NO: 2, amino acid 117-126 of SEQ ID NO: 2, amino acids 19-43 of SEQ ID NO: 3, amino acids 51-69 of SEQ ID NO: 3, and amino acids 77-114 of SEQ ID NO: 3, amino acids 126-136 of SEQ ID NO: 3.

In embodiments of the second aspect, the first antibody and/or the second antibody binds to the epitope as bound by an antibody comprising FRs selected from the group consisting of amino acids 21-46 of SEQ ID NO: 4, amino acids 53-69 of SEQ ID NO: 4, amino acids 73-108 of SEQ ID NO: 4, amino acids 118-127 of SEQ ID NO: 4, amino acids 20-44 of SEQ ID NO: 5, amino acids 53-69 of SEQ ID NO: 5, and amino acids 78-115 of SEQ ID NO: 5, amino acids 127-137 of SEQ ID NO: 5. In particular embodiments, the first antibody and/or the second antibody binds to the epitope as bound by an antibody comprising FRs consisting of amino acids 21-46 of SEQ ID NO: 4, amino acids 53-69 of SEQ ID NO: 4, amino acids 73-108 of SEQ ID NO: 4, amino acids 118-127 of SEQ ID NO: 4, amino acids 20-44 of SEQ ID NO: 5, amino acids 53-69 of SEQ ID NO: 5, and amino acids 78-115 of SEQ ID NO: 5, amino acids 127-137 of SEQ ID NO: 5.

In particular embodiments, the first antibody and/or the second antibody binds to the epitope as bound by an antibody comprising CDRs consisting of amino acids 46-51 of SEQ ID NO: 2, amino acids 69-71 of SEQ ID NO: 2, amino acids 108-116 of SEQ ID NO: 2, amino acids 44-52 of SEQ ID NO: 3, amino acids 70-76 of SEQ ID NO: 3, and amino acids 115-125 of SEQ ID NO: 3; and comprising FRs consisting of amino acids 20-45 of SEQ ID NO: 2, amino acids 52-68 of SEQ ID NO: 2, amino acids 72-107 of SEQ ID NO: 2, amino acid 117-126 of SEQ ID NO: 2, amino acids 19-43 of SEQ ID NO: 3, amino acids 53-69 of SEQ ID NO: 3, and amino acids 77-114 of SEQ ID NO: 3, amino acids 126-136 of SEQ ID NO: 3.

In particular embodiments, the first antibody and/or the second antibody binds to the epitope as bound by an antibody comprising CDRs consisting of amino acids 47-52 of SEQ ID NO: 4, amino acids 70-72 of SEQ ID NO: 4, amino acids 109-117 of SEQ ID NO: 4, amino acids 45-52 of SEQ ID NO: 5, amino acids 70-77 of SEQ ID NO: 5, and amino acids 116-126 of SEQ ID NO: 5, and comprising FRs consisting of amino acids 21-46 of SEQ ID NO: 4, amino acids 53-69 of SEQ ID NO: 4, amino acids 73-108 of SEQ ID NO: 4, amino acids 118-127 of SEQ ID NO: 4, amino acids 20-44 of SEQ ID NO: 5, amino acids 53-69 of SEQ ID NO: 5, and amino acids 78-115 of SEQ ID NO: 5, amino acids 127-137 of SEQ ID NO: 5.

In embodiments of the second aspect, the first and/or the second antibody comprises an amino acid sequences selected from the group consisting of SEQ ID NO: 2, 3, 4 and 5.

In embodiments of the second aspect, the first and/or the second antibody comprises a light chain having an amino acid sequences selected from the group consisting of SEQ ID NO: 2 and 4.

In embodiments of the second aspect, the first and/or the second antibody comprises a heavy chain having an amino acid sequences selected from the group consisting of SEQ ID NO: 3 and 5.

In embodiments of the second aspect, the first or the second antibody comprises a light chain having an amino acid sequence of SEQ ID NO: 2 and a heavy chain having an amino acid sequences of SEQ ID NO: 3.

In embodiments of the second aspect, the first or the second antibody comprises a light chain having an amino acid sequence of SEQ ID NO: 4 and a heavy chain having an amino acid sequences of SEQ ID NO: 5.

In embodiments of the second aspect, one of the first antibody or the second antibody is detectably labeled. Said label may be a molecule detectable by spectroscopic, photochemical, biochemical, immunochemical, chemical, or other physical means. In particular embodiments, the first or the second antibody may be labeled with a fluorescent dye, electron-dense reagent, enzyme (e.g., as commonly used in an ELISA), biotin, digoxigenin, or hapten and other entities which are or can be made detectable. In particular embodiments, the first or second antibody is biotinylated or ruthenylated. Methods for labeling of an antibody are well-known to the person skilled in the art and abundantly described e.g. in Haugland (2003) Molecular Probes Handbook of Fluorescent Probes and Research Chemicals, Molecular Probes, Inc.; Brinkley (1992) Bioconjugate Chem. 3:2; Garman, (1997) Non-Radioactive Labeling: A Practical Approach, Academic Press, London; Means (1990) Bioconjugate Chem. 1:2; Glazer et al Chemical Modification of Proteins. Laboratory Techniques in Biochemistry and Molecular Biology (T. S. Work and E. Work, Eds.) American Elsevier Publishing Co., New York; Lundblad, R. L. and Noyes, C. M. (1984) Chemical Reagents for Protein Modification, Vols. I and II, CRC Press, New York; Pfleiderer, G. (1985) “Chemical Modification of Proteins”, Modern Methods in Protein Chemistry, H. Tschesche, Ed., Walter DeGruyter, Berlin and New York; and Wong (1991) Chemistry of Protein Conjugation and Cross-linking, CRC Press, Boca Raton, Fla.); DeLeon-Rodriguez et al, Chem. Eur. J. 10 (2004) 1149-1155; Lewis et al, Bioconjugate Chem. 12 (2001) 320-324; Li et al, Bioconjugate Chem. 13 (2002) 110-115; Mier et al Bioconjugate Chem. 16 (2005) 240-237.

In particular embodiments, one of the first antibody or the second antibody is capable of binding to a solid phase or is bound to a solid phase.

In particular embodiments, the first antibody is capable of binding to a solid phase or is bound to a solid phase, and the second antibody or antigen-binding fragment thereof, is detectably labeled.

In further embodiments of the second aspect, the first antibody is bound to a solid phase or is capable of binding to a solid phase and has a light chain comprising or consisting of a sequence according to SEQ ID NO: 2, and the second antibody is detectably labeled and has a light chain comprising or consisting of a sequence according to SEQ ID NO: 4.

In further embodiments of the second aspect, the first antibody is bound to a solid phase or is capable of binding to a solid phase and has a light chain comprising or consisting of a sequence according to SEQ ID NO: 4, and the second antibody is detectably labeled and has a light chain comprising or consisting of a sequence according to SEQ ID NO: 2.

In further embodiments of the second aspect, the first antibody is bound to a solid phase or is capable of binding to a solid phase and has a heavy chain comprising or consisting of a sequence according to SEQ ID NO: 3, and the second antibody is detectably labeled and has a heavy chain comprising or consisting of a sequence according to SEQ ID NO: 5.

In further embodiments of the second aspect, the first antibody is bound to a solid phase or is capable of binding to a solid phase and has a heavy chain comprising or consisting of a sequence according to SEQ ID NO: 5, and the second antibody is detectably labeled and has a heavy chain comprising or consisting of a sequence according to SEQ ID NO: 3.

In further embodiments of the second aspect, the first antibody is bound to a solid phase or is capable of binding to a solid phase and has a light chain comprising or consisting of a sequence according to SEQ ID NO: 2 and a heavy chain comprising or consisting of a sequence according to SEQ ID NO: 3, and the second antibody is detectably labeled and has a light chain comprising or consisting of a sequence according to SEQ ID NO: 4 and a heavy chain comprising or consisting of a sequence according to SEQ ID NO: 5.

In further embodiments of the second aspect, the first antibody is bound to a solid phase or is capable of binding to a solid phase and has a light chain comprising or consisting of a sequence according to SEQ ID NO: 4 and a heavy chain comprising or consisting of a sequence according to SEQ ID NO: 5, and the second antibody is detectably labeled and has a light chain comprising or consisting of a sequence according to SEQ ID NO: 2 and a heavy chain comprising or consisting of a sequence according to SEQ ID NO: 3.

In further embodiments, the solid phase is in a form selected from the group consisting of beads, tubes, discs of microplates, and any other surface suitable, in particular suitable for conducting an immunoassay. In particular embodiments, the beads are microbeads. Microbeads are microparticle with a diameter in the nanometer and micrometer range. In embodiments, the microparticles may have a diameter of 50 nanometers to 50 micrometers. In particular, the microparticles have a diameter of between 100 nm and 10 μm, in particular of 200 nm to 5 μm, or of 750 nm to 5 μm. Microparticles comprise or consist of any suitable material known to the person skilled in the art, e.g. they comprise or consist of or essentially consist of inorganic or organic material. In particular, they comprise or consist of or essentially consist of metal or an alloy of metals, or an organic material, or comprise or consist of or essentially consist of carbohydrate elements. In particular embodiments, the material of the microparticles is selected from the group consisting of agarose, polystyrene, latex, polyvinyl alcohol, silica and ferromagnetic metals, alloys or composition materials. Microparticles may also comprise or consist of magnetic or ferromagnetic metals, alloys or compositions. The material may have specific properties such as e.g. being hydrophobic, or hydrophilic. In particular embodiments, the microparticles are dispersed in aqueous solutions and retain a small negative surface charge keeping the microparticles separated and avoiding non-specific clustering.

In particular embodiments, the magnetic or paramagnetic microparticles are separated by magnetic forces. Magnetic forces are applied to pull the paramagnetic or magnetic particles out of the solution/suspension and to retain them as desired while liquid of the solution/suspension can be removed and the particles can e.g. be washed.

In particular embodiments, the first or the second antibody is an IgG antibody. In particular embodiments, the first or the second antibody or the antigen-binding fragment(s) thereof, is an IgG2 antibody. In particular embodiments, the first or the second antibody is an IgG2b antibody, or an antigen-binding fragment thereof, in particular an IgG2b-F(ab′)₂ fragment.

In particular embodiments, the first and/or the second antibody is/are comprised in a physiological solution, in particular in a physiological buffer. In particular embodiments, the buffer is selected from the group of TAPS, Bicine, Tris, Tricine, TAPSO, HEPES, TES, MOPS, PIPES, Cacodylate, and MES. In particular embodiments, the buffer is an IVIES buffer. In particular embodiments, the IVIES buffer comprises the following components: 50 mM IVIES, 150 mM NaCl, 2 mM EDTA-Na₂ (dihydrate), 0.1% N-Methylisothiazolon-HCl, 0.1% Oxypyrion, 0.1% Polydocanol (Thesit), 1.0% Albumin RPLA 4 assay quality, 0.2% PAK<->R-IgG(DET), Millipore-water, pH adjusted to 6.30 with 2N NaOH.

In particular embodiments, the kit of the second aspect as disclosed above is for use in a method of measuring the level of VEGF-A in the presence of a VEGF-A antagonist, as disclosed above with regard to the first aspect. Accordingly, in particular embodiments, the kit of the second aspect as disclosed above is for use in a method for measuring the level of VEGF-A in the presence of a VEGF-A antagonist, the method comprising: incubating a sample with said first and said second antibody, wherein said first and said second antibody are both capable of binding to VEGF-A in the presence of the VEGF-A antagonist and wherein the binding of said first and of said second antibody does not interfere with each other, wherein one of said antibodies is bound to or capable of binding to a solid phase and wherein the other of said antibodies is detectably labeled, thereby forming a detectably labeled complex comprising the first antibody, VEGF-A, and the second antibody, and detecting the complex formed, thereby measuring the level of VEGF-A in the presence of a VEGF-A antagonist.

In particular embodiments, the VEGF-A is human VEGF-A or a variant thereof In particular embodiments, the VEGF-A comprises an amino acid sequence according to SEQ ID NO: 1 or a variant thereof. In particular embodiments, the VEGF-A consists of an amino acid sequence according to SEQ ID NO: 1 or a variant thereof. In particular embodiments, the variant of VEGF-A has the same functionality as VEGF-A, i.e. the variant is a functional variant. In particular embodiments, the variant of VEGF-A exhibits at least 80% sequence identity with the amino acid sequence of human VEGF-A, in particular according to SEQ ID NO: 1. In particular embodiments, the variant of VEGF-A exhibits at least 85%, 90%, 95%, 98% or 99% sequence identity with the amino acid sequence of human VEGF-A, in particular according to SEQ ID NO: 1. In particular embodiments, the variant of VEGF-A exhibits at least 85% or at least 95% sequence identity with the amino acid sequence of human VEGF-A, in particular according to SEQ ID NO: 1. In particular embodiments, the variant of VEGF-A exhibits 85%, 95%, or 98% sequence identity with the amino acid sequence of human VEGF-A, in particular according to SEQ ID NO: 1. In embodiments of the present invention, VEGF-A is present as monomer or as dimer, in particular as homodimer.

In particular embodiments, the VEGF-A is a human VEGF-A isoform or a variant thereof In particular embodiments, the VEGF-A isoform is the human VEGF-A isoform VEGF₁₂₁, VEGF₁₄₅, VEGF₁₆₅, VEGF₁₈₉ and/or VEGF₂₀₆, or a variant thereof In particular embodiments, the variant of the VEGF-A isoform has the same functionality as the respective VEGF-A isoform, i.e. the isoform variant is a functional isoform variant. In particular embodiments, the variant of the VEGF-A isoform exhibits at least 80% sequence identity with the amino acid sequence of the respective human VEGF-A isoform. In particular embodiments, the variant of a VEGF-A isoform exhibits at least 80% sequence identity with the amino acid sequence of human VEGF-A isoform. In particular embodiments, the variant of an VEGF-A isoform exhibits at least 85%, 90%, 95%, 98% or 99% sequence identity with the amino acid sequence of the respective human VEGF-A isoform. In particular embodiments, the variant of a VEGF-A isoform exhibits at least 85% or at least 95% sequence identity with the amino acid sequence of the respective human VEGF-A isoform. In particular embodiments, the variant of a VEGF-A isoform exhibits 85%, 95%, or 98% sequence identity with the amino acid sequence of the respective human VEGF-A isoform. Thus, in particular embodiments, the variant of the VEGF₁₂₁ isoform of VEGF-A exhibits 85%, 95%, or 98% sequence identity with the amino acid sequence of the human VEGF₁₂₁ isoform; the variant of the VEGF₁₄₅ isoform of VEGF-A exhibits 85%, 95%, or 98% sequence identity with the amino acid sequence of the human VEGF₁₄₅ isoform; the variant of the VEGF₁₆₅ isoform of VEGF-A exhibits 85%, 95%, or 98% sequence identity with the amino acid sequence of the human VEGF₁₆₅ isoform, the variant of the VEGF₁₈₉ isoform of VEGF-A exhibits 85%, 95%, or 98% sequence identity with the amino acid sequence of the human VEGF₁₈₉ isoform, and the variant of the VEGF₂₀₆ isoform of VEGF-A exhibits 85%, 95%, or 98% sequence identity with the amino acid sequence of the human VEGF₂₀₆ isoform.

In particular embodiments, the VEGF-A is a human VEGF-A fragment or a variant thereof In particular embodiments, the VEGF-A fragment is the human VEGF-A 110-amino acid fragment or a variant thereof In particular embodiments, the variant of the VEGF-A fragment has the same functionality as the respective VEGF-A fragment, i.e. the fragment variant is a functional fragment variant. In particular embodiments, the variant of the VEGF-A fragment exhibits at least 80% sequence identity with the amino acid sequence of the respective human VEGF-A isoform. In particular embodiments, the variant of a VEGF-A fragment exhibits at least 80% sequence identity with the amino acid sequence of human VEGF-A fragment. In particular embodiments, the variant of an VEGF-A fragment exhibits at least 85%, 90%, 95%, 98% or 99% sequence identity with the amino acid sequence of the respective human VEGF-A fragment. In particular embodiments, the variant of a VEGF-A fragment exhibits at least 85% or at least 95% sequence identity with the amino acid sequence of the respective human VEGF-A fragment. In particular embodiments, the variant of a VEGF-A fragment exhibits 85%, 95%, or 98% sequence identity with the amino acid sequence of the respective human VEGF-A fragment. Thus, in particular embodiments, the variant of the 110-amino acid fragment of VEGF -A exhibits 85%, 95%, or 98% sequence identity with the amino acid sequence of the human 110-amino acid VEGF-A fragment.

In embodiments, the VEGF-A antagonist prevents the interaction between VEGF-A and one or more of the VEGF receptor(s). In particular, the VEGF-A antagonist competes with VEGF-A at binding sites of the receptor or alters the binding site of VEGF-A for its receptor in a manner that it is not able to bind its receptor anymore, or is not able to trigger the functional action anymore which is normally cause by its binding. Accordingly, the VEGF-A antagonist may either bind to an epitope of VEGF-A and thereby hinder the binding of VEGF-A to its receptor, or the VEGF-A antagonist may bind to an epitope of the receptor and thereby prevent the binding of VEGF-A to the receptor. In particular embodiments, the VEGF-A antagonist binds to an epitope on VEGF-A and thereby prevents its binding to VEGF receptors. In particular embodiments, the VEGF-A receptor(s) is/are VEGFA-R1 and/or VEGFA-R2.

In further embodiments of the second aspect of the present invention, the VEGF-A antagonist is selected from the group consisting of a polypeptide, a peptibody, an immunoadhesin, a small molecule and an aptamer.

In particular embodiments, wherein the antagonist is a polypeptide, said polypeptide is an antibody. In a particular embodiment, the antibody is an anti-VEGF-A antibody. In particular, the anti-VEGF antibody is an antibody which binds to VEGF-A with sufficient affinity and specificity. In embodiments, the antibody has a sufficient binding affinity for VEGF-A. In particular, the antibody, or the antigen-binding fragment thereof, binds hVEGF-A with a K_{d} value of between 100 nM-1 pM, i.e. with a Kd value of 100 nM, 50 nM, 1 nM, 900 pM, 800 pM, 700 pm, 600 pM, 500 pM, 400 pM, 300 pM, 200 pM, 100 pM, 50 pM, or 1 pM. In particular embodiments, the antibody, or the antigen-binding fragment thereof, binds human VEGF-A (hVEGF-A) with a K_{d} value of between 50 nM-50 pM, 1 nM-100 pM, or 700 pM-300 pM.

In particular embodiments, the antagonistic VEGF-A antibody is monoclonal or polyclonal. In particular embodiments, the antagonistic antibody for VEGF-A is recombinantly produced. In further embodiments, the antagonistic VEGF-A antibody is a chimeric antibody, in particular a humanized anti-VEGF-A antibody. In particular embodiments, the antagonistic VEGF-A antibody comprises a mutated human IgG1 framework regions. The antagonistic VEGF-A antibody further comprises an antigen-binding complementarity-determining regions (CDR) from the murine anti-hVEGF monoclonal antibody A.4.6.1 that blocks binding of human VEGF to its receptors. In particular embodiments, 93% of the amino acid sequence of the antagonistic VEGF-A antibody, including most of the framework region, are derived from human IgG1, and about 7% of the sequence is derived from the murine antibody A4.6.1 (AVASTIN®). In particular embodiments, the antagonistic VEGF-A antibody is glycosylated. In further embodiments, the antagonistic VEGF-A antibody has a molecular mass of about 149,000 daltons. In particular embodiments, the antagonistic VEGF-A antibody is Bevacizumab (BV), also known as “rhuMAb VEGF” or “AVASTIN®”, which is a recombinant humanized anti-VEGF monoclonal antibody generated according to Presta et al. (1997) Cancer Res. 57:4593-4599.

In particular embodiments, the antagonistic VEGF-A antibody is an antibody fragment. The antibody fragment is selected from the group consisting of Fab-fragment, Fab′-fragment, F(ab′)2-fragment, single domain antibodies (sdAb), nanobodies, single chain Fv (scFv), divalent single-chain variable fragments (di-scFvs), tandem scFvs, diabodies, bispecific diabodies, single chain diabodies (scDb), Bi-specific T-cell engagers (BiTEs), and DART molecules. In particular embodiments, the antagonistic antibody fragment is a Fab-fragment or a F(ab′)2-fragment, in particular a humanized Fab fragment or a humanized or a F(ab′)2-fragment.

In further embodiments, the VEGF-A antagonist is selected from the group consisting of VEGF-Trap, Mucagen, PTK787, SU11248, AG-013736, Bay 439006 (sorafenib), ZD-6474, CP632, CP-547632, AZD-2171, CDP-171, SU-14813, CHIR-258, AEE-788, SB786034, BAY579352, CDP-791, EG-3306, GW-786034, RWJ-417975/CT6758 and KRN-633.

In embodiments of the second aspect, the sample is derived from or is body fluid, in particular selected from the group consisting of whole blood, blood serum, blood plasma, urine, saliva and sputum. In particular embodiments, the sample is derived from or is a whole blood sample, blood serum, or blood plasma.

In embodiments, the sample is derived from a healthy individual or from a patient. In particular embodiments, the patient suffers from a proliferative disorder, in particular from cancer, in particular from metastatic cancers. In particular embodiments, the patient suffers from cancer, in particular from metastatic cancers, and is treated with a VEGF-A antagonist. In particular embodiments, the patient suffers from cancer, in particular from metastatic cancers, and is treated with Bevacizumab.

In particular embodiments, the cancer is selected from the group consisting of carcinoma, lymphoma, blastoma, sarcoma, and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including, e.g., gastrointestinal cancer), pancreatic cancer (including, e.g., metastic pancreatic cancer), glioblastoma, cervical cancer, ovarian cancer, liver cancer, bladder cancer, hepatoma, breast cancer (including locally advanced, recurrent or metastatic HER-2 negative breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. In particular, the patient suffers from cancer selected from the group consisting of colon cancer, lung cancers, renal cancers, ovarian cancers, and glioblastoma multiforme of the brain.

In particular embodiments, the patient is a mammal, reptile, bird or fish. In particular embodiments, the patient is mammal selected from the group consisting of mouse, rat, rabbit, or zebrafish guinea pig, rabbit, horse, donkey, cow, sheep, goat, pig, chicken, camel, cat, dog, turtle, tortoise, snake, lizard, goldfish and primates. In particular, the patient is a human being.

In further embodiments, the method for measuring the level of VEGF-A in the presence of a VEGF-A antagonist for which the kit of the second aspect is used, is an immunoassay, in particular a sandwich immuno-assay, wherein an antibody—antigen—antibody complex, also called a sandwich, is formed. The skilled artisan will appreciate that in a sandwich assay for the detection of VEGF-A, the first antibody may act as a capture antibody and the second antibody may act as a tracer antibody. Alternatively, the second antibody may act as a capture antibody and the first antibody may act as a tracer antibody.

In particular embodiments of measuring the level of VEGF-A in a sample, the first and second antibody are mixed with the sample to be analyzed.

In one embodiment, wherein a sandwich assay is performed without washing step, such mixing/incubation is performed in a single reaction vessel. The sequence of adding and mixing the three ingredients (e.g. microparticles coated with first antibody, or antigen-binding fragment thereof, sample, second detectably-labeled antibody, or antigen-binding fragment thereof, respectively) is not critical. This mixture is incubated for a period of time sufficient for the first antibody (in particular the first antibody coated onto the microparticles) and the detectably labeled second antibody, to bind to VEGF-A.

In another embodiment, wherein a sandwich assay is performed with a washing step, the adding and mixing of the first antibody (in particular the first antibody coated onto microparticles), sample and detectably-labeled second antibody, or antigen-binding fragment(s) thereof, is performed sequentially into a single reaction vessel. In a first step (the analyte-capturing step the microparticles coated with the first antibody are incubated with the sample to be analyzed for a period of time sufficient for the analyte, i.e. VEGF-A, to be bound. Following a washing step, the detectably-labeled second antibody is added and incubated for a period of time sufficient for the second antibody to bind to the analyte, i.e. VEGF-A. In embodiments, the method of the first aspect is practiced in a competitive assay format.

In embodiments, the mixture in incubated for less than 60 min, i.e. less than 60, 55, 50, 45, 40, 35, 30, 25, 20, 15, 10, or 5 min. In particular embodiments, the mixture is incubated for 4 min to 1 hour (i.e. 4, 5, 6, 7, 8, 9, 10, 15, 20, 25, 30, 35, 40, 45, 50, 55, or 60 min). In particular embodiments, the mixture is incubated for 5 min to 45 min, i.e. for 5, 6, 7, 8, 9, 10, 15, 20, 25, 30, 35, 40, or 45 min). In particular embodiments, the mixture is incubated for 5 min to 30 min, i.e. for 5, 6, 7, 8, 9, 10, 15, 20, 25, or 30 min. In particular embodiments, the mixture is incubated for 9 or 18 min. In embodiments, the mixture is incubated for 1-12 hours (i.e. 1, 2, 3, 4, 5, 6, 7, 8, 9, 10, 11, or 12 hours). In particular embodiments, the mixture is incubated for 1-4 hours or for 8-12 hours.

In further embodiments, the mixture is incubated at a temperature of 3-40° C. (i.e. 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40° C.). In particular the mixture is incubated for 3° C. to 8° C. (i.e. 3, 4, 5, 6, 7 or 8), in particular at 4-5° C., or at 20° C. to 25° C. (i.e. at 20, 21, 22, 23, 24, or 25° C.), in particular 20-22° C., or at 35-37° C.

It is well-known to the person skilled in the art that incubation temperature and incubation time depend upon each other. Accordingly, in particular embodiments, the mixture is incubated at 20-25° C. for 10 min to 1 hours, i.e. the mixture is incubated at 20, 21, 22, 23, 24, or 25° C. for 10, 15, 20, 25, 30, 35, 40, 45, 50, 55, or 60 min. In particular embodiments, the mixture is incubated for less than 10 min or less than 20 min at 22° C. In embodiments, the mixture is incubated for 1-12 hours at 3-8° C. In particular, the mixture is incubated for 1-4 hours or for 8-12 hours at 3-8° C., in particular at 4-5° C.

The first and/or the second antibody are incubated for a period of time sufficient for the first antibody coated onto the microparticles and the detectably labeled second antibody, to bind to VEGF-A in the sample.

In particular embodiments, the first and/or the second antibody is/are comprised in and/or are incubated in a physiological solution, in particular in a physiological buffer. In particular embodiments, the buffer is selected from the group of TAPS, Bicine, Tris, Tricine, TAPSO, HEPES, TES, MOPS, PIPES, Cacodylate, and MES. In particular embodiments, the buffer is an MES buffer. In particular embodiments, the IVIES buffer comprises the following components: 50 mM MES, 150 mM NaCl, 2 mM EDTA-Na₂ (dihydrate), 0.1% N-Methylisothiazolon-HCl, 0.1% Oxypyrion, 0.1% Polydocanol (Thesit), 1.0% Albumin RPLA 4 assay quality, 0.2% PAK<->R-IgG(DET), Millipore-water, pH adjusted to 6.30 with 2N NaOH.

In particular embodiments, the formed antibody—antigen—antibody complex, in particular the complex formed comprising the first antibody, VEGF-A—the second antibody, is detected via any method well-known in the art. In particular embodiments, the complex formed is detected via electrochemiluminescent, chemiluminescent, or fluorescence.

In a third aspect, the present invention relates to a composition of matter comprising a first and a second antibody. In particular, said first and said second antibodies are capable of binding to VEGF-A in the presence of a VEGF-A antagonist. In particular, the binding of said first and of said second antibody does not interfere with each other. In particular, one of said antibodies is bound to or capable of binding to a solid phase and wherein the other of said antibodies is detectably labeled. Accordingly, in a third aspect, the present invention relates to a composition of matter comprising a first antibody and a second antibody wherein said first and said second antibody are capable of binding to VEGF-A in the presence of an VEGF-A antagonist, wherein the binding of said first and of said second antibody does not interfere with each other, and wherein one of said antibodies is bound to or capable of binding to a solid phase and wherein the other of said antibodies is detectably labeled.

In particular embodiments, the first and the second antibody do not interfere with each other. Accordingly, the binding of one of these antibodies does not prevent or diminish the binding of the respective other antibody. In particular embodiments of the third aspect, the composition of matter comprises a first antibody against VEGF-A and a second antibody against VEGF-A wherein said first antibody and said second antibody both bind to VEGF-A at identical or at different epitopes. In embodiments of the present invention, the first and second antibody bind to two different epitopes on the same monomer and/or to two different epitopes on each monomer of a dimer. Alternatively, the first and the second antibody bind to the same, or substantially the same epitope, on different monomers of a homodimer. In particular embodiments, said first antibody and said second antibody both bind to VEGF-A at different epitopes.

In particular embodiments, the first and the second antibody individually of each other, bind to the same or to a different epitope as the VEGF-A antagonist, in particular to a different epitope as the antagonistic antibody. In case the first or the second antibody binds to the same epitope as the antagonist, in particular the antagonistic antibody, it is envisaged that the first or second antibody binds the epitope with a lower Kd value than the antagonist. In particular embodiments, the first or second antibody binds the epitope with a Kd value of below 1.5 nM, in particular below 1 nM, below 0.75 nM, in particular below 0.5 nM.

In particular embodiments, the first antibody and the second antibody individually of each other, bind to an epitope which is covered by or bound by a VEGF receptor, in particular by the VEGF-A receptor VEGFA-R1 and/or VEGFA-R2. Thus, said first antibody and said second antibodies, individually of each other, bind to the identical epitope as a VEGF-A receptor, in particular as VEGFA-R1 or VEGFA-R2. Alternatively, said first antibody and said second antibodies, individually of each other, bind to an epitope which is not directly bound by the VEGF-A receptor, such as e.g. VEGFA-R1 or VEGFA-R2, but which is covered by the receptors such that the binding of the first and/or second antibody prevents the binding of the VEGF-A receptor(s). Accordingly, in particular embodiments, the first antibody and/or the second antibody compete for the binding of VEGFA-R1 and/or VEGFA-R2.

VEGF-A is present as monomer or as dimer, in particular as homodimer. Accordingly, in embodiments of the third aspect, the first and second antibody bind to two different epitopes on the same monomer and/or to two different epitopes on each monomer of the dimer. Alternatively, the first and the second antibody bind to the same, or substantially the same epitope, on different monomers of the homodimer.

In embodiments of the third aspect, either the first antibody or the second antibody binds to the epitope as bound by an antibody comprising CDRs selected from the group consisting of amino acids 46-51 of SEQ ID NO: 2, amino acids 69-71 of SEQ ID NO: 2, amino acids 108-116 of SEQ ID NO: 2, amino acids 44-52 of SEQ ID NO: 3, amino acids 70-76 of SEQ ID NO: 3, amino acids 115-125 of SEQ ID NO: 3, amino acids 47-52 of SEQ ID NO: 4, amino acids 70-72 of SEQ ID NO: 4, amino acids 109-117 of SEQ ID NO: 4, amino acids 45-52 of SEQ ID NO: 5, amino acids 70-77 of SEQ ID NO: 5, and amino acids 116-126 of SEQ ID NO: 5.

In embodiments of the third aspect, the first antibody and/or the second antibody binds to the epitope as bound by an antibody comprising CDRs selected from the group consisting of amino acids 46-51 of SEQ ID NO: 2, amino acids 69-71 of SEQ ID NO: 2, amino acids 108-116 of SEQ ID NO: 2, amino acids 44-52 of SEQ ID NO: 3, amino acids 70-76 of SEQ ID NO: 3, and amino acids 115-125 of SEQ ID NO: 3. In particular embodiments, the first antibody and/or the second antibody binds to the epitope as bound by an antibody comprising the CDRs consisting of amino acids 46-51 of SEQ ID NO: 2, amino acids 69-71 of SEQ ID NO: 2, amino acids 108-116 of SEQ ID NO: 2, amino acids 44-52 of SEQ ID NO: 3, amino acids 70-76 of SEQ ID NO: 3, and amino acids 115-125 of SEQ ID NO: 3.

In embodiments of the third aspect, the first antibody and/or the second antibody binds to the epitope as bound by an antibody comprising CDRs selected from the group consisting of amino acids 47-52 of SEQ ID NO: 4, amino acids 70-72 of SEQ ID NO: 4, amino acids 109-117 of SEQ ID NO: 4, amino acids 45-52 of SEQ ID NO: 5, amino acids 70-77 of SEQ ID NO: 5, and amino acids 116-126 of SEQ ID NO: 5. In particular embodiments, the first antibody and/or the second antibody binds to the epitope as bound by an antibody comprising CDRs consisting of amino acids 47-52 of SEQ ID NO: 4, amino acids 70-72 of SEQ ID NO: 4, amino acids 109-117 of SEQ ID NO: 4, amino acids 45-52 of SEQ ID NO: 5, amino acids 70-77 of SEQ ID NO: 5, and amino acids 116-126 of SEQ ID NO: 5.

In embodiments of the third aspect, the first antibody and/or the second antibody binds to the epitope as bound by an antibody comprising FR selected from the group consisting of amino acids 20-45 of SEQ ID NO: 2, amino acids 52-68 of SEQ ID NO: 2, amino acids 72-107 of SEQ ID NO: 2, amino acid 117-126 of SEQ ID NO: 2, amino acids 19-43 of SEQ ID NO: 3, amino acids 53-69 of SEQ ID NO: 3, and amino acids 77-114 of SEQ ID NO: 3, amino acids 126-136 of SEQ ID NO: 3. In particular embodiments, the first antibody and/or the second antibody binds to the epitope as bound by an antibody comprising the FRs consisting of amino acids 20-45 of SEQ ID NO: 2, amino acids 51-68 of SEQ ID NO: 2, amino acids 72-107 of SEQ ID NO: 2, amino acid 117-126 of SEQ ID NO: 2, amino acids 19-43 of SEQ ID NO: 3, amino acids 51-69 of SEQ ID NO: 3, and amino acids 77-114 of SEQ ID NO: 3, amino acids 126-136 of SEQ ID NO: 3.

In embodiments of the third aspect, the first antibody and/or the second antibody binds to the epitope as bound by an antibody comprising FRs selected from the group consisting of amino acids 21-46 of SEQ ID NO: 4, amino acids 53-69 of SEQ ID NO: 4, amino acids 73-108 of SEQ ID NO: 4, amino acids 118-127 of SEQ ID NO: 4, amino acids 20-44 of SEQ ID NO: 5, amino acids 53-69 of SEQ ID NO: 5, and amino acids 78-115 of SEQ ID NO: 5, amino acids 127-137 of SEQ ID NO: 5. In particular embodiments, the first antibody and/or the second antibody binds to the epitope as bound by an antibody comprising FRs consisting of amino acids 21-46 of SEQ ID NO: 4, amino acids 53-69 of SEQ ID NO: 4, amino acids 73-108 of SEQ ID NO: 4, amino acids 118-127 of SEQ ID NO: 4, amino acids 20-44 of SEQ ID NO: 5, amino acids 53-69 of SEQ ID NO: 5, and amino acids 78-115 of SEQ ID NO: 5, amino acids 127-137 of SEQ ID NO: 5.

In particular embodiments, the first antibody and/or the second antibody binds to the epitope as bound by an antibody comprising CDRs consisting of amino acids 46-51 of SEQ ID NO: 2, amino acids 69-71 of SEQ ID NO: 2, amino acids 108-116 of SEQ ID NO: 2, amino acids 44-52 of SEQ ID NO: 3, amino acids 70-76 of SEQ ID NO: 3, and amino acids 115-125 of SEQ ID NO: 3; and comprising FRs consisting of amino acids 20-45 of SEQ ID NO: 2, amino acids 52-68 of SEQ ID NO: 2, amino acids 72-107 of SEQ ID NO: 2, amino acid 117-126 of SEQ ID NO: 2, amino acids 19-43 of SEQ ID NO: 3, amino acids 53-69 of SEQ ID NO: 3, and amino acids 77-114 of SEQ ID NO: 3, amino acids 126-136 of SEQ ID NO: 3.

In particular embodiments, the first antibody and/or the second antibody binds to the epitope as bound by an antibody comprising CDRs consisting of amino acids 47-52 of SEQ ID NO: 4, amino acids 70-72 of SEQ ID NO: 4, amino acids 109-117 of SEQ ID NO: 4, amino acids 45-52 of SEQ ID NO: 5, amino acids 70-77 of SEQ ID NO: 5, and amino acids 116-126 of SEQ ID NO: 5, and comprising FRs consisting of amino acids 21-46 of SEQ ID NO: 4, amino acids 53-69 of SEQ ID NO: 4, amino acids 73-108 of SEQ ID NO: 4, amino acids 118-127 of SEQ ID NO: 4, amino acids 20-44 of SEQ ID NO: 5, amino acids 53-69 of SEQ ID NO: 5, and amino acids 78-115 of SEQ ID NO: 5, amino acids 127-137 of SEQ ID NO: 5.

In embodiments of the third aspect, the first and/or the second antibody comprises an amino acid sequences selected from the group consisting of SEQ ID NO: 2, 3, 4 and 5.

In embodiments of the third aspect, the first and/or the second antibody comprises a light chain having an amino acid sequences selected from the group consisting of SEQ ID NO: 2 and 4.

In embodiments of the third aspect, the first and/or the second antibody comprises a heavy chain having an amino acid sequences selected from the group consisting of SEQ ID NO: 3 and 5.

In embodiments of the third aspect, the first or the second antibody comprises a light chain having an amino acid sequence of SEQ ID NO: 2 and a heavy chain having an amino acid sequences of SEQ ID NO: 3.

In embodiments of the third aspect, the first or the second antibody comprises a light chain having an amino acid sequence of SEQ ID NO: 4 and a heavy chain having an amino acid sequences of SEQ ID NO: 5.

In embodiments of the third aspect, one of the first antibody or the second antibody is detectably labeled. Said label may be a molecule detectable by spectroscopic, photochemical, biochemical, immunochemical, chemical, or other physical means. In particular embodiments, the first or the second antibody may be labeled with a fluorescent dye, electron-dense reagent, enzyme (e.g., as commonly used in an ELISA), biotin, digoxigenin, or hapten and other entities which are or can be made detectable. In particular embodiments, the first or second antibody is biotinylated or ruthenylated. Methods for labeling of an antibody are well-known to the person skilled in the art and abundantly described e.g. in Haugland (2003) Molecular Probes Handbook of Fluorescent Probes and Research Chemicals, Molecular Probes, Inc.; Brinkley (1992) Bioconjugate Chem. 3:2; Garman, (1997) Non-Radioactive Labeling: A Practical Approach, Academic Press, London; Means (1990) Bioconjugate Chem. 1:2; Glazer et al Chemical Modification of Proteins. Laboratory Techniques in Biochemistry and Molecular Biology (T. S. Work and E. Work, Eds.) American Elsevier Publishing Co., New York; Lundblad, R. L. and Noyes, C. M. (1984) Chemical Reagents for Protein Modification, Vols. I and II, CRC Press, New York; Pfleiderer, G. (1985) “Chemical Modification of Proteins”, Modern Methods in Protein Chemistry, H. Tschesche, Ed., Walter DeGruyter, Berlin and New York; and Wong (1991) Chemistry of Protein Conjugation and Cross-linking, CRC Press, Boca Raton, Fla.); DeLeon-Rodriguez et al, Chem. Eur. J. 10 (2004) 1149-1155; Lewis et al, Bioconjugate Chem. 12 (2001) 320-324; Li et al, Bioconjugate Chem. 13 (2002) 110-115; Mier et al Bioconjugate Chem. 16 (2005) 240-237.

In particular embodiments, one of the first antibody or the second antibody is capable of binding to a solid phase or is bound to a solid phase.

In particular embodiments, the first antibody is capable of binding to a solid phase or is bound to a solid phase, and the second antibody is detectably labeled.

In further embodiments of the third aspect, the first antibody is bound to a solid phase or is capable of binding to a solid phase and has a light chain comprising or consisting of a sequence according to SEQ ID NO: 2, and the second antibody is detectably labeled and has a light chain comprising or consisting of a sequence according to SEQ ID NO: 4.

In further embodiments of the third aspect, the first antibody is bound to a solid phase or is capable of binding to a solid phase and has a light chain comprising or consisting of a sequence according to SEQ ID NO: 4, and the second antibody is detectably labeled and has a light chain comprising or consisting of a sequence according to SEQ ID NO: 2.

In further embodiments of the third aspect, the first antibody is bound to a solid phase or is capable of binding to a solid phase and has a heavy chain comprising or consisting of a sequence according to SEQ ID NO: 3, and the second antibody is detectably labeled and has a heavy chain comprising or consisting of a sequence according to SEQ ID NO: 5.

In further embodiments of the third aspect, the first antibody is bound to a solid phase or is capable of binding to a solid phase and has a heavy chain comprising or consisting of a sequence according to SEQ ID NO: 5, and the second antibody is detectably labeled and has a heavy chain comprising or consisting of a sequence according to SEQ ID NO: 3.

In further embodiments of the third aspect, the first antibody is bound to a solid phase or is capable of binding to a solid phase and has a light chain comprising or consisting of a sequence according to SEQ ID NO: 2 and a heavy chain comprising or consisting of a sequence according to SEQ ID NO: 3, and the second antibody is detectably labeled and has a light chain comprising or consisting of a sequence according to SEQ ID NO: 4 and a heavy chain comprising or consisting of a sequence according to SEQ ID NO: 5.

In further embodiments of the third aspect, the first antibody is bound to a solid phase or is capable of binding to a solid phase and has a light chain comprising or consisting of a sequence according to SEQ ID NO: 4 and a heavy chain comprising or consisting of a sequence according to SEQ ID NO: 5, and the second antibody is detectably labeled and has a light chain comprising or consisting of a sequence according to SEQ ID NO: 2 and a heavy chain comprising or consisting of a sequence according to SEQ ID NO: 3.

In further embodiments, the solid phase is in a form selected from the group consisting of beads, tubes, discs of microplates, and any other surface suitable, in particular suitable for conducting an immunoassay. In particular embodiments, the beads are microbeads. Microbeads are microparticle with a diameter in the nanometer and micrometer range. In embodiments, the microparticles may have a diameter of 50 nanometers to 50 micrometers. In particular, the microparticles have a diameter of between 100 nm and 10 μm, in particular of 200 nm to 5 μm, or of 750 nm to 5 μm. Microparticles comprise or consist of any suitable material known to the person skilled in the art, e.g. they comprise or consist of or essentially consist of inorganic or organic material. In particular, they comprise or consist of or essentially consist of metal or an alloy of metals, or an organic material, or comprise or consist of or essentially consist of carbohydrate elements. In particular embodiments, the material of the microparticles is selected from the group consisting of agarose, polystyrene, latex, polyvinyl alcohol, silica and ferromagnetic metals, alloys or composition materials. Microparticles may also comprise or consist of magnetic or ferromagnetic metals, alloys or compositions. The material may have specific properties such as e.g. being hydrophobic, or hydrophilic. In particular embodiments, the microparticles are dispersed in aqueous solutions and retain a small negative surface charge keeping the microparticles separated and avoiding non-specific clustering.

In particular embodiments, the magnetic or paramagnetic microparticles are separated by magnetic forces. Magnetic forces are applied to pull the paramagnetic or magnetic particles out of the solution/suspension and to retain them as desired while liquid of the solution/suspension can be removed and the particles can e.g. be washed.

In particular embodiments, the first or the second antibody is an IgG antibody. In particular embodiments, the first or the second antibody is an IgG2 antibody. In particular embodiments, the first or the second antibody is an IgG2b antibody, or an antigen-binding fragment thereof, in particular an IgG2b-F(ab′)₂ fragment.

In particular embodiments, the composition of matter further comprises a VEGF-A antagonist.

In embodiments, the VEGF-A antagonist prevents the interaction between VEGF-A and one or more of the VEGF receptor(s). In particular, the VEGF-A antagonist competes with VEGF-A at binding sites of the receptor or alters the binding site of VEGF-A for its receptor in a manner that it is not able to bind its receptor anymore, or is not able to trigger the functional action anymore which is normally cause by its binding. Accordingly, the VEGF-A antagonist may either bind to an epitope of VEGF-A and thereby hinder the binding of VEGF-A to its receptor, or the VEGF-A antagonist may bind to an epitope of the receptor and thereby prevent the binding of VEGF-A to the receptor. In particular embodiments, the VEGF-A antagonist binds to an epitope on VEGF-A and thereby prevents its binding to VEGF receptors. In particular embodiments, the VEGF-A receptor(s) is/are VEGFA-R1 and/or VEGFA-R2.

In further embodiments of the third aspect of the present invention, the VEGF-A antagonist is selected from the group consisting of a polypeptide, a peptibody, an immunoadhesin, a small molecule and an aptamer.

In particular embodiments, wherein the antagonist is a polypeptide, said polypeptide is an antibody. In a particular embodiment, the antibody is an anti-VEGF-A antibody. In particular, the anti-VEGF antibody, is an antibody, or an antigen-binding fragment thereof, which binds to VEGF-A with sufficient affinity and specificity. In embodiments, the antibody, or the antigen-binding fragment thereof, has a sufficient binding affinity for VEGF-A. In particular, the antibody, or the antigen-binding fragment thereof, binds hVEGF-A with a K_{d} value of between 100 nM-1 pM, i.e. with a Kd value of 100 nM, 50 nM, 1 nM, 900 pM, 800 pM, 700 pm, 600 pM, 500 pM, 400 pM, 300 pM, 200 pM, 100 pM, 50 pM, or 1 pM. In particular embodiments, the antibody, or the antigen-binding fragment thereof, binds human VEGF-A (hVEGF-A) with a K_{d} value of between 50 nM-50 pM, 1 nM-100 pM, or 700 pM-300 pM.

In particular embodiments, the antagonistic VEGF-A antibody is monoclonal or polyclonal. In particular embodiments, the antagonistic antibody, or the antigen-binding fragment thereof, for VEGF-A is recombinantly produced. In further embodiments, the antagonistic VEGF-A antibody is a chimeric antibody in particular, a humanized anti-VEGF-A antibody. In particular embodiments, the antagonistic VEGF-A antibody comprises a mutated human IgG1 framework regions. The antagonistic VEGF-A antibody further comprises an antigen-binding complementarity-determining regions (CDR) from the murine anti-hVEGF monoclonal antibody A.4.6.1 that blocks binding of human VEGF to its receptors. In particular embodiments, 93% of the amino acid sequence of the antagonistic VEGF-A antibody, including most of the framework region, are derived from human IgG1, and about 7% of the sequence is derived from the murine antibody A4.6.1 (AVASTIN®). In particular embodiments, the antagonistic VEGF-A antibody is glycosylated. In further embodiments, the antagonistic VEGF-A antibody has a molecular mass of about 149,000 daltons. In particular embodiments, the antagonistic VEGF-A antibody is Bevacizumab (BV), also known as “rhuMAb VEGF” or “AVASTIN®”, which is a recombinant humanized anti-VEGF monoclonal antibody generated according to Presta et al. (1997) Cancer Res. 57:4593-4599.

In particular embodiments, the antagonistic VEGF-A antibody is an antibody fragment. The antibody fragment is selected from the group consisting of Fab-fragment, Fab′-fragment, F(ab′)2-fragment, single domain antibodies (sdAb), nanobodies, single chain Fv (scFv), divalent single-chain variable fragments (di-scFvs), tandem scFvs, diabodies, bispecific diabodies, single chain diabodies (scDb), Bi-specific T-cell engagers (BiTEs), and DART molecules. In particular embodiments, the antagonistic antibody fragment is a Fab-fragment or a F(ab′)₂-fragment, in particular a humanized Fab fragment or a humanized or a F(ab′)₂-fragment.

In further embodiments, the VEGF-A antagonist is selected from the group consisting of VEGF-Trap, Mucagen, PTK787, SU11248, AG-013736, Bay 439006 (sorafenib), ZD-6474, CP632, CP-547632, AZD-2171, CDP-171, SU-14813, CHIR-258, AEE-788, SB786034, BAY579352, CDP-791, EG-3306, GW-786034, RWJ-417975/CT6758 and KRN-633.

In particular embodiments, the first and/or the second antibody and/or the VEGF-A antagonist is/are comprised in a physiological solution, in particular in a physiological buffer. In particular embodiments, the buffer is selected from the group of TAPS, Bicine, Tris, Tricine, TAPSO, HEPES, TES, MOPS, PIPES, Cacodylate, and IVIES. In particular embodiments, the buffer is an MES buffer. In particular embodiments, the MES buffer comprises the following components: 50 mM IVIES, 150 mM NaCl, 2 mM EDTA-Na₂ (dihydrate), 0.1% N-Methylisothiazolon-HCl, 0.1% Oxypyrion, 0.1% Polydocanol (Thesit), 1.0% Albumin RPLA 4 assay quality, 0.2% PAK<->R-IgG(DET), Millipore-water, pH adjusted to 6.30 with 2N NaOH.

In particular embodiments, the composition of matter as disclosed above is for use in a method of measuring the level of VEGF-A in the presence of a VEGF-A antagonist, as disclosed above with regard to the first aspect. Accordingly, in particular embodiments, the composition of matter as disclosed above is for use in a method for measuring the level of VEGF-A in the presence of a VEGF-A antagonist, the method comprising: incubating a sample with said first and said second antibody, wherein said first and said second antibody are both capable of binding to VEGF-A in the presence of the VEGF-A antagonist and wherein the binding of said first and of said second antibody does not interfere with each other, wherein one of said antibodies is bound to or capable of binding to a solid phase and wherein the other of said antibodies is detectably labeled, thereby forming a detectably labeled complex comprising the first antibody, VEGF-A, and the second antibody, and detecting the complex formed, thereby measuring the level of VEGF-A in the presence of a VEGF-A antagonist.

In particular embodiments, the VEGF-A is human VEGF-A or a variant thereof In particular embodiments, the VEGF-A comprises an amino acid sequence according to SEQ ID NO: 1 or a variant thereof. In particular embodiments, the VEGF-A consists of an amino acid sequence according to SEQ ID NO: 1 or a variant thereof. In particular embodiments, the variant of VEGF-A has the same functionality as VEGF-A, i.e. the variant is a functional variant. In particular embodiments, the variant of VEGF-A exhibits at least 80% sequence identity with the amino acid sequence of human VEGF-A, in particular according to SEQ ID NO: 1. In particular embodiments, the variant of VEGF-A exhibits at least 85%, 90%, 95%, 98% or 99% sequence identity with the amino acid sequence of human VEGF-A, in particular according to SEQ ID NO: 1. In particular embodiments, the variant of VEGF-A exhibits at least 85% or at least 95% sequence identity with the amino acid sequence of human VEGF-A, in particular according to SEQ ID NO: 1. In particular embodiments, the variant of VEGF-A exhibits 85%, 95%, or 98% sequence identity with the amino acid sequence of human VEGF-A, in particular according to SEQ ID NO: 1. In embodiments of the present invention, VEGF-A is present as monomer or as dimer, in particular as homodimer.

In particular embodiments, the VEGF-A is a human VEGF-A isoform or a variant thereof In particular embodiments, the VEGF-A isoform is the human VEGF-A isoform VEGF₁₂₁, VEGF₁₄₅, VEGF₁₆₅, VEGF₁₈₉ and/or VEGF₂₀₆, or a variant thereof. In particular embodiments, the variant of the VEGF-A isoform has the same functionality as the respective VEGF-A isoform, i.e. the isoform variant is a functional isoform variant. In particular embodiments, the variant of the VEGF-A isoform exhibits at least 80% sequence identity with the amino acid sequence of the respective human VEGF-A isoform. In particular embodiments, the variant of a VEGF-A isoform exhibits at least 80% sequence identity with the amino acid sequence of human VEGF-A isoform. In particular embodiments, the variant of an VEGF-A isoform exhibits at least 85%, 90%, 95%, 98% or 99% sequence identity with the amino acid sequence of the respective human VEGF-A isoform. In particular embodiments, the variant of a VEGF-A isoform exhibits at least 85% or at least 95% sequence identity with the amino acid sequence of the respective human VEGF-A isoform. In particular embodiments, the variant of a VEGF-A isoform exhibits 85%, 95%, or 98% sequence identity with the amino acid sequence of the respective human VEGF-A isoform. Thus, in particular embodiments, the variant of the VEGF₁₂₁ isoform of VEGF -A exhibits 85%, 95%, or 98% sequence identity with the amino acid sequence of the human VEGF₁₂₁ isoform; the variant of the VEGF₁₄₅ isoform of VEGF-A exhibits 85%, 95%, or 98% sequence identity with the amino acid sequence of the human VEGF₁₄₅ isoform; the variant of the VEGF₁₆₅ isoform of VEGF-A exhibits 85%, 95%, or 98% sequence identity with the amino acid sequence of the human VEGF₁₆₅ isoform, the variant of the VEGF₁₈₉ isoform of VEGF-A exhibits 85%, 95%, or 98% sequence identity with the amino acid sequence of the human VEGF₁₈₉ isoform, and the variant of the VEGF₂₀₆ isoform of VEGF-A exhibits 85%, 95%, or 98% sequence identity with the amino acid sequence of the human VEGF₂₀₆ isoform.

In particular embodiments, the VEGF-A is a human VEGF-A fragment or a variant thereof In particular embodiments, the VEGF-A fragment is the human VEGF-A 110-amino acid fragment or a variant thereof In particular embodiments, the variant of the VEGF-A fragment has the same functionality as the respective VEGF-A fragment, i.e. the fragment variant is a functional fragment variant. In particular embodiments, the variant of the VEGF-A fragment exhibits at least 80% sequence identity with the amino acid sequence of the respective human VEGF-A isoform. In particular embodiments, the variant of a VEGF-A fragment exhibits at least 80% sequence identity with the amino acid sequence of human VEGF-A fragment. In particular embodiments, the variant of an VEGF-A fragment exhibits at least 85%, 90%, 95%, 98% or 99% sequence identity with the amino acid sequence of the respective human VEGF-A fragment. In particular embodiments, the variant of a VEGF-A fragment exhibits at least 85% or at least 95% sequence identity with the amino acid sequence of the respective human VEGF-A fragment. In particular embodiments, the variant of a VEGF-A fragment exhibits 85%, 95%, or 98% sequence identity with the amino acid sequence of the respective human VEGF-A fragment. Thus, in particular embodiments, the variant of the 110-amino acid fragment of VEGF -A exhibits 85%, 95%, or 98% sequence identity with the amino acid sequence of the human 110-amino acid VEGF-A fragment.

In embodiments, the VEGF-A antagonist prevents the interaction between VEGF-A and one or more of the VEGF receptor(s). In particular, the VEGF-A antagonist competes with VEGF-A at binding sites of the receptor or alters the binding site of VEGF-A for its receptor in a manner that it is not able to bind its receptor anymore, or is not able to trigger the functional action anymore which is normally cause by its binding. Accordingly, the VEGF-A antagonist may either bind to an epitope of VEGF-A and thereby hinder the binding of VEGF-A to its receptor, or the VEGF-A antagonist may bind to an epitope of the receptor and thereby prevent the binding of VEGF-A to the receptor. In particular embodiments, the VEGF-A antagonist binds to an epitope on VEGF-A and thereby prevents its binding to VEGF receptors. In particular embodiments, the VEGF-A receptor(s) is/are VEGFA-R1 and/or VEGFA-R2.

In further embodiments of the third aspect of the present invention, the VEGF-A antagonist is selected from the group consisting of a polypeptide, a peptibody, an immunoadhesin, a small molecule and an aptamer.

In particular embodiments, wherein the antagonist is a polypeptide, said polypeptide is an antibody. In a particular embodiment, the antibody is an anti-VEGF-A antibody. In particular, the anti-VEGF antibody is an antibody which binds to VEGF-A with sufficient affinity and specificity. In embodiments, the antibody has a sufficient binding affinity for VEGF-A. In particular, the antibody, or the antigen-binding fragment thereof, binds hVEGF-A with a K_{d} value of between 100 nM-1 pM, i.e. with a Kd value of 100 nM, 50 nM, 1 nM, 900 pM, 800 pM, 700 pm, 600 pM, 500 pM, 400 pM, 300 pM, 200 pM, 100 pM, 50 pM, or 1 pM. In particular embodiments, the antibody, or the antigen-binding fragment thereof, binds human VEGF-A (hVEGF-A) with a K_{d} value of between 50 nM-50 pM, 1 nM-100 pM, or 700 pM-300 pM.

In particular embodiments, the antagonistic VEGF-A antibody is monoclonal or polyclonal. In particular embodiments, the antagonistic antibody for VEGF-A is recombinantly produced. In further embodiments, the antagonistic VEGF-A antibody is a chimeric antibody, in particular a humanized anti-VEGF-A antibody. In particular embodiments, the antagonistic VEGF-A antibody comprises a mutated human IgG1 framework regions. The antagonistic VEGF-A antibody further comprises an antigen-binding complementarity-determining regions (CDR) from the murine anti-hVEGF monoclonal antibody A.4.6.1 that blocks binding of human VEGF to its receptors. In particular embodiments, 93% of the amino acid sequence of the antagonistic VEGF-A antibody, including most of the framework region, are derived from human IgG1, and about 7% of the sequence is derived from the murine antibody A4.6.1 (AVASTIN®). In particular embodiments, the antagonistic VEGF-A antibody is glycosylated. In further embodiments, the antagonistic VEGF-A antibody has a molecular mass of about 149,000 daltons. In particular embodiments, the antagonistic VEGF-A antibody is Bevacizumab (BV), also known as “rhuMAb VEGF” or “AVASTIN®”, which is a recombinant humanized anti-VEGF monoclonal antibody generated according to Presta et al. (1997) Cancer Res. 57:4593-4599.

In particular embodiments, the antagonistic VEGF-A antibody is an antibody fragment. The antibody fragment is selected from the group consisting of Fab-fragment, Fab′-fragment, F(ab′)2-fragment, single domain antibodies (sdAb), nanobodies, single chain Fv (scFv), divalent single-chain variable fragments (di-scFvs), tandem scFvs, diabodies, bispecific diabodies, single chain diabodies (scDb), Bi-specific T-cell engagers (BiTEs), and DART molecules. In particular embodiments, the antagonistic antibody fragment is a Fab-fragment or a F(ab′)₂-fragment, in particular a humanized Fab fragment or a humanized or a F(ab′)₂-fragment.

In further embodiments, the VEGF-A antagonist is selected from the group consisting of VEGF-Trap, Mucagen, PTK787, SU11248, AG-013736, Bay 439006 (sorafenib), ZD-6474, CP632, CP-547632, AZD-2171, CDP-171, SU-14813, CHIR-258, AEE-788, SB786034, BAY579352, CDP-791, EG-3306, GW-786034, RWJ-417975/CT6758 and KRN-633.

In embodiments of the third aspect, the sample is derived from or is body fluid, in particular selected from the group consisting of whole blood, blood serum, blood plasma, urine, saliva and sputum. In particular embodiments, the sample is derived from or is a whole blood sample, blood serum, or blood plasma.

In embodiments, the sample is derived from a healthy individual or from a patient. In particular embodiments, the patient suffers from a proliferative disorder, in particular from cancer, in particular from metastatic cancers. In particular embodiments, the patient suffers from cancer, in particular from metastatic cancers, and is treated with a VEGF-A antagonist. In particular embodiments, the patient suffers from cancer, in particular from metastatic cancers, and is treated with Bevacizumab.

In particular embodiments, the cancer is selected from the group consisting of carcinoma, lymphoma, blastoma, sarcoma, and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including, e.g., gastrointestinal cancer), pancreatic cancer (including, e.g., metastic pancreatic cancer), glioblastoma, cervical cancer, ovarian cancer, liver cancer, bladder cancer, hepatoma, breast cancer (including locally advanced, recurrent or metastatic HER-2 negative breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. In particular, the patient suffers from cancer selected from the group consisting of colon cancer, lung cancers, renal cancers, ovarian cancers, and glioblastoma multiforme of the brain.

In particular embodiments, the patient is a mammal, reptile, bird or fish. In particular embodiments, the patient is mammal selected from the group consisting of mouse, rat, rabbit, or zebrafish guinea pig, rabbit, horse, donkey, cow, sheep, goat, pig, chicken, camel, cat, dog, turtle, tortoise, snake, lizard, goldfish and primates. In particular, the patient is a human being.

In further embodiments, the method for measuring the level of VEGF-A in the presence of a VEGF-A antagonist for which the composition of matter as disclosed above is used, is an immunoassay, in particular a sandwich immuno-assay, wherein an antibody—antigen—antibody complex, also called a sandwich, is formed. The skilled artisan will appreciate that in a sandwich assay for the detection of VEGF-A, the first antibody may act as a capture antibody and the second antibody may act as a tracer antibody. Alternatively, the second antibody may act as a capture antibody and the first antibody may act as a tracer antibody.

In particular embodiments of measuring the level of VEGF-A in a sample, the first and second antibody are mixed with the sample to be analyzed.

In one embodiment, wherein a sandwich assay is performed without washing step, such mixing/incubation is performed in a single reaction vessel. The sequence of adding and mixing the three ingredients (e.g. microparticles coated with first antibody, or antigen-binding fragment thereof, sample, second detectably-labeled antibody, or antigen-binding fragment thereof, respectively) is not critical. This mixture is incubated for a period of time sufficient for the first antibody (in particular the first antibody coated onto the microparticles) and the detectably labeled second antibody, to bind to VEGF-A.

In another embodiment, wherein a sandwich assay is performed with a washing step, the adding and mixing of the first antibody (in particular the first antibody coated onto microparticles), sample and detectably-labeled second antibody, or antigen-binding fragment(s) thereof, is performed sequentially into a single reaction vessel. In a first step (the analyte-capturing step) the microparticles coated with the first antibody are incubated with the sample to be analyzed for a period of time sufficient for the analyte, i.e. VEGF-A, to be bound. Following a washing step, the detectably-labeled second antibody is added and incubated for a period of time sufficient for the second antibody to bind to the analyte, i.e. VEGF-A. In embodiments, the method of the first aspect is practiced in a competitive assay format.

In embodiments, the mixture in incubated for less than 60 min, i.e. less than 60, 55, 50, 45, 40, 35, 30, 25, 20, 15, 10, or 5 min. In particular embodiments, the mixture is incubated for 4 min to 1 hour (i.e. 4, 5, 6, 7, 8, 9, 10, 15, 20, 25, 30, 35, 40, 45, 50, 55, or 60 min). In particular embodiments, the mixture is incubated for 5 min to 45 min, i.e. for 5, 6, 7, 8, 9, 10, 15, 20, 25, 30, 35, 40, or 45 min). In particular embodiments, the mixture is incubated for 5 min to 30 min, i.e. for 5, 6, 7, 8, 9, 10, 15, 20, 25, or 30 min. In particular embodiments, the mixture is incubated for 9 or 18 min. In embodiments, the mixture is incubated for 1-12 hours (i.e. 1, 2, 3, 4, 5, 6, 7, 8, 9, 10, 11, or 12 hours). In particular embodiments, the mixture is incubated for 1-4 hours or for 8-12 hours.

In further embodiments, the mixture is incubated at a temperature of 3-40° C. (i.e. 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40° C.). In particular the mixture is incubated for 3° C. to 8° C. (i.e. 3, 4, 5, 6, 7 or 8), in particular at 4-5° C., or at 20° C. to 25° C. (i.e. at 20, 21, 22, 23, 24, or 25° C.), in particular 20-22° C., or at 35-37° C.

It is well-known to the person skilled in the art that incubation temperature and incubation time depend upon each other. Accordingly, in particular embodiments, the mixture is incubated at 20-25° C. for 10 min to 1 hours, i.e. the mixture is incubated at 20, 21, 22, 23, 24, or 25° C. for 10, 15, 20, 25, 30, 35, 40, 45, 50, 55, or 60 min. In particular embodiments, the mixture is incubated for less than 10 min or less than 20 min at 22° C. In embodiments, the mixture is incubated for 1-12 hours at 3-8° C. In particular, the mixture is incubated for 1-4 hours or for 8-12 hours at 3-8° C., in particular at 4-5° C.

The first and/or the second antibody are incubated for a period of time sufficient for the first antibody coated onto the microparticles and the detectably labeled second antibody, to bind to VEGF-A in the sample.

In particular embodiments, the first and/or the second antibody is/are comprised in and/or are incubated in a physiological solution, in particular in a physiological buffer. In particular embodiments, the buffer is selected from the group of TAPS, Bicine, Tris, Tricine, TAPSO, HEPES, TES, MOPS, PIPES, Cacodylate, and MES. In particular embodiments, the buffer is an MES buffer. In particular embodiments, the IVIES buffer comprises the following components: 50 mM MES, 150 mM NaCl, 2 mM EDTA-Na₂ (dihydrate), 0.1% N-Methylisothiazolon-HCl, 0.1% Oxypyrion, 0.1% Polydocanol (Thesit), 1.0% Albumin RPLA 4 assay quality, 0.2% PAK<->R-IgG(DET), Millipore-water, pH adjusted to 6.30 with 2N NaOH.

In particular embodiments, the formed antibody—antigen—antibody complex, in particular the complex formed comprising the first antibody, VEGF-A—the second antibody, is detected via any method well-known in the art. In particular embodiments, the complex formed is detected via electrochemiluminescent, chemiluminescent, or fluorescence.

In a fourth aspect, the present invention relates to a method of detecting a protein complex comprising human VEGF-A as a first protein and a non-human or chimeric protein as a second protein, comprising the steps of

(a) incubating a sample comprising said complex with an detectably-labeled antibody, which is able to bind to or is binding to VEGF-A and/or to the second non-human or chimeric protein of said complex, and

(b) detecting the detectably-labeled antibody bound to the complex.

In particular embodiments, the VEGF-A is human VEGF-A or a variant thereof. In particular embodiments, the VEGF-A comprises an amino acid sequence according to SEQ ID NO: 1 or a variant thereof. In particular embodiments, the VEGF-A consists of an amino acid sequence according to SEQ ID NO: 1 or a variant thereof. In particular embodiments, the variant of VEGF-A has the same functionality as VEGF-A, i.e. the variant is a functional variant. In particular embodiments, the variant of VEGF-A exhibits at least 80% sequence identity with the amino acid sequence of human VEGF-A, in particular according to SEQ ID NO: 1. In particular embodiments, the variant of VEGF-A exhibits at least 85%, 90%, 95%, 98% or 99% sequence identity with the amino acid sequence of human VEGF-A, in particular according to SEQ ID NO: 1. In particular embodiments, the variant of VEGF-A exhibits at least 85% or at least 95% sequence identity with the amino acid sequence of human VEGF-A, in particular according to SEQ ID NO: 1. In particular embodiments, the variant of VEGF-A exhibits 85%, 95%, or 98% sequence identity with the amino acid sequence of human VEGF-A, in particular according to SEQ ID NO: 1.

In particular embodiments of the fourth aspect, the non-human or chimeric protein is an antibody which binds to VEGF-A.

Accordingly, in particular embodiments, the complex to be detected comprises human VEGF-A or a variant thereof and a non-human or chimeric antibody, which binds to VEGF-A.

In particular embodiments, the detectably-labeled antibody or the antigen-binding fragment thereof binds to VEGF-A.

In embodiments, wherein the detectably-labeled antibody binds to VEGF-A, the non-human or chimeric antibody and the detectably-labeled antibody bind to VEGF-A at identical or at different epitopes. In particular embodiments, the non-human or chimeric antibody and the detectably-labeled antibody bind to VEGF-A at different epitopes.

In particular embodiments, the non-human or chimeric antibody and the detectably-labeled antibody do not interfere with each other. Accordingly, the binding of one of these antibodies does not prevent or diminish the binding of the respective other antibody. In embodiments of the present invention, the non-human or chimeric antibody and the detectably-labeled antibody bind to two different epitopes on the same monomer and/or to two different epitopes on each monomer of a dimer. Alternatively, the non-human or chimeric antibody and the detectably-labeled antibody bind to the same, or substantially the same epitope, on different monomers of a homodimer. In particular embodiments, the non-human or chimeric antibody and the detectably-labeled antibody bind to VEGF-A at different epitopes.

In particular embodiments, the non-human or chimeric antibody and the detectably-labeled antibody individually of each other, bind to the same or to a different epitope as the VEGF-A antagonist, in particular to a different epitope as the antagonistic antibody. In case the non-human or chimeric antibody and the detectably-labeled antibody bind to the same epitope as the antagonist, in particular the antagonistic antibody, it is envisaged that the non-human or chimeric antibody and the detectably-labeled antibody bind the epitope with a lower Kd value than the antagonist. In particular embodiments, the non-human or chimeric antibody and the detectably-labeled antibody binds the epitope with a Kd value of below 1.5 nM, in particular below 1 nM, below 0.75 nM, in particular below 0.5 nM.

In particular embodiments, the non-human or chimeric antibody and the detectably-labeled antibody, individually of each other, bind to an epitope which is covered by or bound by a VEGF receptor, in particular by the VEGF-A receptor VEGFA-R1 and/or VEGFA-R2. Thus, said non-human or chimeric antibody and the detectably-labeled antibody bind to the identical epitope as a VEGF-A receptor, in particular as VEGFA-R1 or VEGFA-R2. Alternatively, the non-human or chimeric antibody and the detectably-labeled antibody bind to an epitope which is not directly bound by the VEGF-A receptor, such as e.g. VEGFA-R1 or VEGFA-R2, but which is covered by the receptors such that the binding of the first and/or second antibody prevents the binding of the VEGF-A receptor(s). Accordingly, in particular embodiments, the non-human or chimeric antibody and the detectably-labeled antibody compete for the binding of VEGFA-R1 and/or VEGFA-R2.

VEGF-A is present as monomer or as dimer, in particular as homodimer. Accordingly, in embodiments of the fourth aspect, the non-human or chimeric antibody and the detectably-labeled antibody bind to two different epitopes on the same monomer and/or to two different epitopes on each monomer of the dimer. Alternatively, the first and the second antibody bind to the same, or substantially the same epitope, on different monomers of the homodimer.

In embodiments of the fourth aspect, either the non-human or chimeric antibody and the detectably-labeled antibody binds to the epitope as bound by an antibody comprising CDRs selected from the group consisting of amino acids 46-51 of SEQ ID NO: 2, amino acids 69-71 of SEQ ID NO: 2, amino acids 108-116 of SEQ ID NO: 2, amino acids 44-52 of SEQ ID NO: 3, amino acids 70-76 of SEQ ID NO: 3, amino acids 115-125 of SEQ ID NO: 3, amino acids 47-52 of SEQ ID NO: 4, amino acids 70-72 of SEQ ID NO: 4, amino acids 109-117 of SEQ ID NO: 4, amino acids 45-52 of SEQ ID NO: 5, amino acids 70-77 of SEQ ID NO: 5, and amino acids 116-126 of SEQ ID NO: 5.

In embodiments of the fourth aspect, the non-human or chimeric antibody and the detectably-labeled antibody binds to the epitope as bound by an antibody comprising CDRs selected from the group consisting of amino acids 46-51 of SEQ ID NO: 2, amino acids 69-71 of SEQ ID NO: 2, amino acids 108-116 of SEQ ID NO: 2, amino acids 44-52 of SEQ ID NO: 3, amino acids 70-76 of SEQ ID NO: 3, and amino acids 115-125 of SEQ ID NO: 3. In particular embodiments, the first antibody and/or the second antibody binds to the epitope as bound by an antibody comprising the CDRs consisting of amino acids 46-51 of SEQ ID NO: 2, amino acids 69-71 of SEQ ID NO: 2, amino acids 108-116 of SEQ ID NO: 2, amino acids 44-52 of SEQ ID NO: 3, amino acids 70-76 of SEQ ID NO: 3, and amino acids 115-125 of SEQ ID NO: 3.

In embodiments of the fourth aspect, the non-human or chimeric antibody and the detectably-labeled antibody binds to the epitope as bound by an antibody comprising CDRs selected from the group consisting of amino acids 47-52 of SEQ ID NO: 4, amino acids 70-72 of SEQ ID NO: 4, amino acids 109-117 of SEQ ID NO: 4, amino acids 45-52 of SEQ ID NO: 5, amino acids 70-77 of SEQ ID NO: 5, and amino acids 116-126 of SEQ ID NO: 5. In particular embodiments, the first antibody and/or the second antibody binds to the epitope as bound by an antibody comprising CDRs consisting of amino acids 47-52 of SEQ ID NO: 4, amino acids 70-72 of SEQ ID NO: 4, amino acids 109-117 of SEQ ID NO: 4, amino acids 45-52 of SEQ ID NO: 5, amino acids 70-77 of SEQ ID NO: 5, and amino acids 116-126 of SEQ ID NO: 5.

In embodiments of the fourth aspect, the non-human or chimeric antibody and the detectably-labeled antibody binds to the epitope as bound by an antibody comprising FR selected from the group consisting of amino acids 20-45 of SEQ ID NO: 2, amino acids 52-68 of SEQ ID NO: 2, amino acids 72-107 of SEQ ID NO: 2, amino acid 117-126 of SEQ ID NO: 2, amino acids 19-43 of SEQ ID NO: 3, amino acids 53-69 of SEQ ID NO: 3, and amino acids 77-114 of SEQ ID NO: 3, amino acids 126-136 of SEQ ID NO: 3. In particular embodiments, the first antibody and/or the second antibody binds to the epitope as bound by an antibody comprising the FRs consisting of amino acids 20-45 of SEQ ID NO: 2, amino acids 51-68 of SEQ ID NO: 2, amino acids 72-107 of SEQ ID NO: 2, amino acid 117-126 of SEQ ID NO: 2, amino acids 19-43 of SEQ ID NO: 3, amino acids 51-69 of SEQ ID NO: 3, and amino acids 77-114 of SEQ ID NO: 3, amino acids 126-136 of SEQ ID NO: 3.

In embodiments of the fourth aspect, the non-human or chimeric antibody and the detectably-labeled antibody binds to the epitope as bound by an antibody comprising FRs selected from the group consisting of amino acids 21-46 of SEQ ID NO: 4, amino acids 53-69 of SEQ ID NO: 4, amino acids 73-108 of SEQ ID NO: 4, amino acids 118-127 of SEQ ID NO: 4, amino acids 20-44 of SEQ ID NO: 5, amino acids 53-69 of SEQ ID NO: 5, and amino acids 78-115 of SEQ ID NO: 5, amino acids 127-137 of SEQ ID NO: 5. In particular embodiments, the first antibody and/or the second antibody binds to the epitope as bound by an antibody comprising FRs consisting of amino acids 21-46 of SEQ ID NO: 4, amino acids 53-69 of SEQ ID NO: 4, amino acids 73-108 of SEQ ID NO: 4, amino acids 118-127 of SEQ ID NO: 4, amino acids 20-44 of SEQ ID NO: 5, amino acids 53-69 of SEQ ID NO: 5, and amino acids 78-115 of SEQ ID NO: 5, amino acids 127-137 of SEQ ID NO: 5.

In particular embodiments, the non-human or chimeric antibody and the detectably-labeled antibody binds to the epitope as bound by an antibody comprising CDRs consisting of amino acids 46-51 of SEQ ID NO: 2, amino acids 69-71 of SEQ ID NO: 2, amino acids 108-116 of SEQ ID NO: 2, amino acids 44-52 of SEQ ID NO: 3, amino acids 70-76 of SEQ ID NO: 3, and amino acids 115-125 of SEQ ID NO: 3; and comprising FRs consisting of amino acids 20-45 of SEQ ID NO: 2, amino acids 52-68 of SEQ ID NO: 2, amino acids 72-107 of SEQ ID NO: 2, amino acid 117-126 of SEQ ID NO: 2, amino acids 19-43 of SEQ ID NO: 3, amino acids 53-69 of SEQ ID NO: 3, and amino acids 77-114 of SEQ ID NO: 3, amino acids 126-136 of SEQ ID NO: 3.

In particular embodiments, the non-human or chimeric antibody and the detectably-labeled antibody binds to the epitope as bound by an antibody comprising CDRs consisting of amino acids 47-52 of SEQ ID NO: 4, amino acids 70-72 of SEQ ID NO: 4, amino acids 109-117 of SEQ ID NO: 4, amino acids 45-52 of SEQ ID NO: 5, amino acids 70-77 of SEQ ID NO: 5, and amino acids 116-126 of SEQ ID NO: 5, and comprising FRs consisting of amino acids 21-46 of SEQ ID NO: 4, amino acids 53-69 of SEQ ID NO: 4, amino acids 73-108 of SEQ ID NO: 4, amino acids 118-127 of SEQ ID NO: 4, amino acids 20-44 of SEQ ID NO: 5, amino acids 53-69 of SEQ ID NO: 5, and amino acids 78-115 of SEQ ID NO: 5, amino acids 127-137 of SEQ ID NO: 5.

In embodiments of the fourth aspect, the non-human or chimeric antibody and the detectably-labeled antibody comprises an amino acid sequences selected from the group consisting of SEQ ID NO: 2, 3, 4 and 5.

In embodiments of the fourth aspect, the non-human or chimeric antibody and the detectably-labeled antibody comprises a light chain having an amino acid sequence of SEQ ID NO: 2 and a heavy chain having an amino acid sequences of SEQ ID NO: 3.

In embodiments of the fourth aspect, the non-human or chimeric antibody and the detectably-labeled antibody comprises a light chain having an amino acid sequence of SEQ ID NO: 4 and a heavy chain having an amino acid sequences of SEQ ID NO: 5.

In embodiments of the fourth aspect, the non-human or chimeric antibody and/or the detectably-labeled antibody is labeled with a molecule detectable by spectroscopic, photochemical, biochemical, immunochemical, chemical, or other physical means. In particular embodiments the non-human or chimeric antibody and/or the detectably-labeled antibody is labeled with a fluorescent dye, electron-dense reagent, enzyme (e.g., as commonly used in an ELISA), biotin, digoxigenin, or hapten and other entities which are or can be made detectable. In particular embodiments, the non-human or chimeric antibody and/or the detectably-labeled antibody is biotinylated or ruthenylated. Methods for labeling of an antibody are well-known to the person skilled in the art and abundantly described e.g. in Haugland (2003) Molecular Probes Handbook of Fluorescent Probes and Research Chemicals, Molecular Probes, Inc.; Brinkley (1992) Bioconjugate Chem. 3:2; Garman, (1997) Non-Radioactive Labeling: A Practical Approach, Academic Press, London; Means (1990) Bioconjugate Chem. 1:2; Glazer et al Chemical Modification of Proteins. Laboratory Techniques in Biochemistry and Molecular Biology (T. S. Work and E. Work, Eds.) American Elsevier Publishing Co., New York; Lundblad, R. L. and Noyes, C. M. (1984) Chemical Reagents for Protein Modification, Vols. I and II, CRC Press, New York; Pfleiderer, G. (1985) “Chemical Modification of Proteins”, Modern Methods in Protein Chemistry, H. Tschesche, Ed., Walter DeGruyter, Berlin and New York; and Wong (1991) Chemistry of Protein Conjugation and Cross-linking, CRC Press, Boca Raton, Fla.); DeLeon-Rodriguez et al, Chem. Eur. J. 10 (2004) 1149-1155; Lewis et al, Bioconjugate Chem. 12 (2001) 320-324; Li et al, Bioconjugate Chem. 13 (2002) 110-115; Mier et al Bioconjugate Chem. 16 (2005) 240-237.

In particular embodiments, the non-human or chimeric antibody and the detectably-labeled antibody is capable of binding to a solid phase or is bound to a solid phase.

In particular embodiments, the non-human or chimeric antibody is capable of binding to a solid phase or is bound to a solid phase.

In further embodiments of the fourth aspect, the non-human or chimeric antibody is bound to a solid phase or is capable of binding to a solid phase and has a light chain comprising or consisting of a sequence according to SEQ ID NO: 2, and the detectably antibody has a light chain comprising or consisting of a sequence according to SEQ ID NO: 4.

In further embodiments of the fourth aspect, the non-human or chimeric antibody is bound to a solid phase or is capable of binding to a solid phase and has a light chain comprising or consisting of a sequence according to SEQ ID NO: 4, and the detectably labeled antibody has a light chain comprising or consisting of a sequence according to SEQ ID NO: 2.

In further embodiments of the fourth aspect, the non-human or chimeric antibody is bound to a solid phase or is capable of binding to a solid phase and has a heavy chain comprising or consisting of a sequence according to SEQ ID NO: 3, and the detectably labeled antibody has a heavy chain comprising or consisting of a sequence according to SEQ ID NO: 5.

In further embodiments of the fourth aspect, the non-human or chimeric antibody is bound to a solid phase or is capable of binding to a solid phase and has a heavy chain comprising or consisting of a sequence according to SEQ ID NO: 5, and the detectably labeled antibody has a heavy chain comprising or consisting of a sequence according to SEQ ID NO: 3.

In further embodiments of the fourth aspect, the non-human or chimeric antibody is bound to a solid phase or is capable of binding to a solid phase and has a light chain comprising or consisting of a sequence according to SEQ ID NO: 2 and a heavy chain comprising or consisting of a sequence according to SEQ ID NO: 3, and the detectably labeled antibody has a light chain comprising or consisting of a sequence according to SEQ ID NO: 4 and a heavy chain comprising or consisting of a sequence according to SEQ ID NO: 5.

In further embodiments of the fourth aspect, the non-human or chimeric antibody is bound to a solid phase or is capable of binding to a solid phase and has a light chain comprising or consisting of a sequence according to SEQ ID NO: 4 and a heavy chain comprising or consisting of a sequence according to SEQ ID NO: 5, and the detectably labeled antibody has a light chain comprising or consisting of a sequence according to SEQ ID NO: 2 and a heavy chain comprising or consisting of a sequence according to SEQ ID NO: 3.

In further embodiments, the solid phase is in a form selected from the group consisting of beads, tubes, discs of microplates, and any other surface suitable, in particular suitable for conducting an immunoassay. In particular embodiments, the beads are microbeads. Microbeads are microparticles with a diameter in the nanometer and micrometer range. In embodiments, the microparticles may have a diameter of 50 nanometers to 50 micrometers. In particular, the microparticles have a diameter of between 100 nm and 10 μm, in particular of 200 nm to 5 μm, or of 750 nm to 5 μm. Microparticles comprise or consist of any suitable material known to the person skilled in the art, e.g. they comprise or consist of or essentially consist of inorganic or organic material. In particular, they comprise or consist of or essentially consist of metal or an alloy of metals, or an organic material, or comprise or consist of or essentially consist of carbohydrate elements. In particular embodiments, the material of the microparticles is selected from the group consisting of agarose, polystyrene, latex, polyvinyl alcohol, silica and ferromagnetic metals, alloys or composition materials. Microparticles may also comprise or consist of magnetic or ferromagnetic metals, alloys or compositions. The material may have specific properties such as e.g. being hydrophobic, or hydrophilic. In particular embodiments, the microparticles are dispersed in aqueous solutions and retain a small negative surface charge keeping the microparticles separated and avoiding non-specific clustering.

In particular embodiments, the magnetic or paramagnetic microparticles are separated by magnetic forces. Magnetic forces are applied to pull the paramagnetic or magnetic particles out of the solution/suspension and to retain them as desired while liquid of the solution/suspension can be removed and the particles can e.g. be washed.

In particular embodiments, the non-human or chimeric antibody and/or the detectably-labeled antibody is an IgG antibody. In particular embodiments, the non-human or chimeric antibody and/or the detectably-labeled antibody is an IgG2 antibody. In particular embodiments, the non-human or chimeric antibody and/or the detectably-labeled antibody is an IgG2b antibody, or an antigen-binding fragment thereof, in particular an IgG2b-F(ab′)₂ fragment.

In particular embodiments of the fourth aspect, the sample comprising said complex incubated in step (a) is also incubated with a VEGF-A antagonist. In particular embodiments, the incubation of the sample comprising said complex with a VEGF-A antagonist is performed prior to, simultaneously with or subsequent to the incubation with the detectably labeled antibody or antigen-binding fragment thereof.

In embodiments, the VEGF-A antagonist prevents the interaction between VEGF-A and one or more of the VEGF receptor(s). In particular, the VEGF-A antagonist competes with VEGF-A at binding sites of the receptor or alters the binding site of VEGF-A for its receptor in a manner that it is not able to bind its receptor anymore, or is not able to trigger the functional action anymore which is normally cause by its binding. Accordingly, the VEGF-A antagonist may either bind to an epitope of VEGF-A and thereby hinder the binding of VEGF-A to its receptor, or the VEGF-A antagonist may bind to an epitope of the receptor and thereby prevent the binding of VEGF-A to the receptor. In particular embodiments, the VEGF-A antagonist binds to an epitope on VEGF-A and thereby prevents its binding to VEGF receptors. In particular embodiments, the VEGF-A receptor(s) is/are VEGFA-R1 and/or VEGFA-R2.

In further embodiments of the fourth aspect, the VEGF-A antagonist is selected from the group consisting of a polypeptide, a peptibody, an immunoadhesin, a small molecule and an aptamer.

In particular embodiments, wherein the antagonist is a polypeptide, said polypeptide is an antibody. In a particular embodiment, the antibody is an anti-VEGF-A antibody,. In particular, the anti-VEGF antibody is an antibody, or an antigen-binding fragment thereof, which binds to VEGF-A with sufficient affinity and specificity. In embodiments, the antibody, or the antigen-binding fragment thereof, has a sufficient binding affinity for VEGF-A. In particular, the antibody, or the antigen-binding fragment thereof, binds hVEGF-A with a K_{d} value of between 100 nM-1 pM, i.e. with a Kd value of 100 nM, 50 nM, 1 nM, 900 pM, 800 pM, 700 pm, 600 pM, 500 pM, 400 pM, 300 pM, 200 pM, 100 pM, 50 pM, or 1 pM. In particular embodiments, the antibody, or the antigen-binding fragment thereof, binds human VEGF-A (hVEGF-A) with a K_{d} value of between 50 nM-50 pM, 1 nM-100 pM, or 700 pM-300 pM.

In particular embodiments, the antagonistic VEGF-A antibody is monoclonal or polyclonal. In particular embodiments, the antagonistic antibody for VEGF-A is recombinantly produced. In further embodiments, the antagonistic VEGF-A antibody is a chimeric antibody in particular, a humanized anti-VEGF-A antibody. In particular embodiments, the antagonistic VEGF-A antibody comprises a mutated human IgG1 framework regions. The antagonistic VEGF-A antibody further comprises an antigen-binding complementarity-determining regions (CDR) from the murine anti-hVEGF monoclonal antibody A.4.6.1 that blocks binding of human VEGF to its receptors. In particular embodiments, 93% of the amino acid sequence of the antagonistic VEGF-A antibody, including most of the framework region, are derived from human IgG1, and about 7% of the sequence is derived from the murine antibody A4.6.1 (AVASTIN®). In particular embodiments, the antagonistic VEGF-A antibody is glycosylated. In further embodiments, the antagonistic VEGF-A antibody has a molecular mass of about 149,000 daltons. In particular embodiments, the antagonistic VEGF-A antibody is Bevacizumab (BV), also known as “rhuMAb VEGF” or “AVASTIN®”, which is a recombinant humanized anti-VEGF monoclonal antibody generated according to Presta et al. (1997) Cancer Res. 57:4593-4599.

In particular embodiments, the antagonistic VEGF-A antibody is an antibody fragment. The antibody fragment is selected from the group consisting of Fab-fragment, Fab′-fragment, F(ab′)2-fragment, single domain antibodies (sdAb), nanobodies, single chain Fv (scFv), divalent single-chain variable fragments (di-scFvs), tandem scFvs, diabodies, bispecific diabodies, single chain diabodies (scDb), Bi-specific T-cell engagers (BiTEs), and DART molecules. In particular embodiments, the antagonistic antibody fragment is a Fab-fragment or a F(ab′)₂-fragment, in particular a humanized Fab fragment or a humanized or a F(ab′)₂-fragment.

In further embodiments, the VEGF-A antagonist is selected from the group consisting of VEGF-Trap, Mucagen, PTK787, SU11248, AG-013736, Bay 439006 (sorafenib), ZD-6474, CP632, CP-547632, AZD-2171, CDP-171, SU-14813, CHIR-258, AEE-788, SB786034, BAY579352, CDP-791, EG-3306, GW-786034, RWJ-417975/CT6758 and KRN-633.

In particular embodiments, the complex, the detectably labeled antibody and/or the VEGF-A antagonist is/are comprised in a physiological solution, in particular in a physiological buffer. In particular embodiments, the buffer is selected from the group of TAPS, Bicine, Tris, Tricine, TAPSO, HEPES, TES, MOPS, PIPES, Cacodylate, and IVIES. In particular embodiments, the buffer is an MES buffer. In particular embodiments, the MES buffer comprises the following components: 50 mM IVIES, 150 mM NaCl, 2 mM EDTA-Na₂ (dihydrate), 0.1% N-Methylisothiazolon-HCl, 0.1% Oxypyrion, 0.1% Polydocanol (Thesit), 1.0% Albumin RPLA 4 assay quality, 0.2% PAK<->R-IgG(DET), Millipore-water, pH adjusted to 6.30 with 2N NaOH.

In particular embodiments of step (a), the detectably labeled antibody is mixed with the sample comprising said complex. This mixture is incubated for a period of time sufficient for the detectably labeled antibody, to bind to the complex.

In embodiments, the mixture in incubated for less than 60 min, i.e. less than 60, 55, 50, 45, 40, 35, 30, 25, 20, 15, 10, or 5 min. In particular embodiments, the mixture is incubated for 4 min to 1 hour (i.e. 4, 5, 6, 7, 8, 9, 10, 15, 20, 25, 30, 35, 40, 45, 50, 55, or 60 min). In particular embodiments, the mixture is incubated for 5 min to 45 min, i.e. for 5, 6, 7, 8, 9, 10, 15, 20, 25, 30, 35, 40, or 45 min). In particular embodiments, the mixture is incubated for 5 min to 30 min, i.e. for 5, 6, 7, 8, 9, 10, 15, 20, 25, or 30 min. In particular embodiments, the mixture is incubated for less than 20 min or for less than 10 min. In particular embodiments, the mixture is incubated for 18 or 9 min.

In embodiments, the mixture is incubated for 1-12 hours (i.e. 1, 2, 3, 4, 5, 6, 7, 8, 9, 10, 11, or 12 hours). In particular embodiments, the mixture is incubated for 1-4 hours or for 8-12 hours.

In further embodiments, the mixture is incubated at a temperature of 3-40° C. (i.e. 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40° C.). In particular the mixture is incubated for 3° C. to 8° C. (i.e. 3, 4, 5, 6, 7 or 8), in particular at 4-5° C., or at 20° C. to 25° C. (i.e. at 20, 21, 22, 23, 24, or 25° C.), in particular 20-22° C., or at 35-37° C.

It is well-known to the person skilled in the art that incubation temperature and incubation time depend upon each other. Accordingly, in particular embodiments, the mixture is incubated at 20-25° C. for 10 min to 1 hours, i.e. the mixture is incubated at 20, 21, 22, 23, 24, or 25° C. for 10, 15, 20, 25, 30, 35, 40, 45, 50, 55, or 60 min. In particular embodiments, the mixture is incubated for less than 10 min or less than 20 min at 22° C. In further embodiments, the mixture is incubated at 3-8° C. for 8-12 hours, i.e. the mixture is incubated at 3, 4, 5, 6, 7, or 8 ° C. for 8, 9, 10, 11, or 12 hours. In particular embodiments, the mixture is incubated for 12 hours at 4° C. In particular, the mixture is incubated for 1-4 hours or for 8-12 hours at 3-8° C., in particular at 4-5° C.

In particular embodiments, the complex and/or the detectably labeled antibody is/are comprised in and/or are incubated in a physiological solution, in particular in a physiological buffer. In particular embodiments, the buffer is selected from the group of TAPS, Bicine, Tris, Tricine, TAPSO, HEPES, TES, MOPS, PIPES, Cacodylate, and IVIES. In particular embodiments, the buffer is an MES buffer. In particular embodiments, the MES buffer comprises the following components: 50 mM IVIES, 150 mM NaCl, 2 mM EDTA-Na₂ (dihydrate), 0.1% N-Methylisothiazolon-HCl, 0.1% Oxypyrion, 0.1% Polydocanol (Thesit), 1.0% Albumin RPLA 4 assay quality, 0.2% PAK<->R-IgG(DET), Millipore-water, pH adjusted to 6.30 with 2N NaOH.

In particular embodiments, the formed antibody—antigen—antibody complex, in particular the complex formed comprising the complex of human VEGF-A and the non-human or chimeric antibody, and the detectably labeled antibody or the antigen-binding fragment thereof, is detected in step (b) via any method well-known in the art. In particular embodiments, the complex formed is detected via electrochemiluminescent, chemiluminescent, or fluorescence.

In particular, the present invention relates to the following items:

1. A method for measuring the level of VEGF-A, in the presence of a VEGF-A antagonist, the method comprising:
incubating a sample with a first and a second antibody,
   wherein said first and said second antibody are capable of binding to VEGF-A or a variant thereof, in the presence of the VEGF-A antagonist, and wherein the binding of said first and of said second antibody does not interfere with each other,
   wherein one of said antibodies is detectably labeled, thereby forming a detectably labeled complex comprising the first antibody, VEGF-A or a variant thereof, and the second antibody, and
detecting the complex formed, thereby measuring the level of VEGF-A in the presence of a VEGF-A antagonist.
   2. The method of item 1, wherein the VEGF-A antagonist is a VEGF-A-binding polypeptide, in particular a VEGF-A-binding antibody.
   3. The method of items 1 or 2, wherein the VEGF-A antagonist is the antibody bevacizumab.
   4. The method of any one of items 1 to 3, wherein either the first and/or the second antibody binds to an epitope which are covered by or bound by a VEGF receptor.
   5. The method of any one of items 1 to 4, wherein either the first or the second antibody comprises CDRs consisting of amino acids 46-51 of SEQ ID NO: 2, amino acids 69-71 of SEQ ID NO: 2, amino acids 108-116 of SEQ ID NO: 2, amino acids 44-52 of SEQ ID NO: 3, amino acids 70-76 of SEQ ID NO: 3, amino acids 115-125 of SEQ ID NO: 3, or
comprises CDRs consisting of amino acids 47-52 of SEQ ID NO: 4, amino acids 70-72 of SEQ ID NO: 4, amino acids 109-117 of SEQ ID NO: 4, amino acids 45-52 of SEQ ID NO: 5, amino acids 70-77 of SEQ ID NO: 5, and amino acids 116-126 of SEQ ID NO: 5.
   6. The method of any one of items 1 to 5, wherein either the first antibody and/or the second antibody binds to the epitope as bound by an antibody comprising CDRs consisting of amino acids 46-51 of SEQ ID NO: 2, amino acids 69-71 of SEQ ID NO: 2, amino acids 108-116 of SEQ ID NO: 2, amino acids 44-52 of SEQ ID NO: 3, amino acids 70-76 of SEQ ID NO: 3, amino acids 115-125 of SEQ ID NO: 3; and comprising FRs consisting of amino acids 20-45 of SEQ ID NO: 2, amino acids 52-68 of SEQ ID NO: 2, amino acids 72-107 of SEQ ID NO: 2, amino acid 117-126 of SEQ ID NO: 2, amino acids 19-43 of SEQ ID NO: 3, amino acids 53-69 of SEQ ID NO: 3, and amino acids 77-114 of SEQ ID NO: 3, amino acids 126-136 of SEQ ID NO: 3, or comprising CDRs consisting of amino acids 47-52 of SEQ ID NO: 4, amino acids 70-72 of SEQ ID NO: 4, amino acids 109-117 of SEQ ID NO: 4, amino acids 45-52 of SEQ ID NO: 5, amino acids 70-77 of SEQ ID NO: 5, and amino acids 116-126 of SEQ ID NO: 5, and comprising FRs consisting of amino acids 21-46 of SEQ ID NO: 4, amino acids 53-69 of SEQ ID NO: 4, amino acids 73-108 of SEQ ID NO: 4, amino acids 118-127 of SEQ ID NO: 4, amino acids 20-44 of SEQ ID NO: 5, amino acids 53-69 of SEQ ID NO: 5, and amino acids 78-115 of SEQ ID NO: 5, amino acids 127-137 of SEQ ID NO: 5.
   7. The method of any one of items 1 to 6, wherein either the first or the second antibody comprises a light chain having an amino acid sequence of SEQ ID NO: 2 and a heavy chain having an amino acid sequences of SEQ ID NO: 3.
   8. The method of any one of items 1 to 6, wherein either the first or the second antibody comprises a light chain having an amino acid sequence of SEQ ID NO: 4 and a heavy chain having an amino acid sequences of SEQ ID NO: 5.
   9. The method according to any one of items 1 to 8, wherein the first or the second antibody is bound to or capable of binding to a solid phase.
   10. The method according to any one of items 1 to 9, wherein the first antibody is bound to or capable of binding to a solid phase and comprises a light chain having an amino acid sequence of SEQ ID NO: 2 and a heavy chain having an amino acid sequences of SEQ ID NO: 3, and wherein the second antibody is detectably labeled and comprises a light chain having an amino acid sequence of SEQ ID NO: 4 and a heavy chain having an amino acid sequences of SEQ ID NO: 5.
   11. The method according to any one of items 1 to 10, wherein one of the first antibody or the second antibody is biotinylated.
   12. The method according to any one of items 1 to 10, wherein one of the first antibody or the second antibody is ruthenylated.
   13. The method according to any of items 1 to 12, wherein the sample is derived from a patient treated with a VEGF-A antagonist.
   14. The method according to any of items 1 to 13, wherein the sample is body fluid, in particular whole blood, serum or plasma.
   15. The method of any o items 1 to 14, wherein the method for measuring the level of VEGF-A or a variant thereof, in the presence of a VEGF-A antagonist is a sandwich immuno assay.
   16. The method of any o items 1 to 15, wherein the method for measuring the level of VEGF-A, in the presence of a VEGF-A antagonist comprises the detection of the formed complex via electrochemiluminscence.
   17. A kit for measuring the level of VEGF-A, in the presence of a VEGF-A antagonist, the kit comprising: a first and a second antibody,
wherein said first and said second antibody both are capable of binding to VEGF-A, in the presence of the VEGF-A antagonist and wherein the binding of said first and of said second antibody does not interfere with each other,
wherein one of said antibodies is detectably labeled.
   18. The kit according to item 17, wherein said kit comprises the antibodies as specified in any one of items 4 to 12.
   19. A composition of matter comprising a first and a second antibody,
wherein said first and said second antibody are both capable of binding to VEGF-A or a variant thereof, in the presence of a VEGF-A antagonist,
wherein the binding of said first and of said second antibody does not interfere with each other, and
wherein one of said antibodies is detectably labeled.
   20. The composition of matter of item 19, wherein either the first and/or the second antibody binds to an epitope which are covered by or bound by a VEGF receptor.
   21. The composition of matter of any one of items 19 or 20, wherein either the first or the second antibody comprises CDRs consisting of amino acids 46-51 of SEQ ID NO: 2, amino acids 69-71 of SEQ ID NO: 2, amino acids 108-116 of SEQ ID NO: 2, amino acids 44-52 of SEQ ID NO: 3, amino acids 70-76 of SEQ ID NO: 3, amino acids 115-125 of SEQ ID NO: 3, or comprises CDRs consisting of amino acids 47-52 of SEQ ID NO: 4, amino acids 70-72 of SEQ ID NO: 4, amino acids 109-117 of SEQ ID NO: 4, amino acids 45-52 of SEQ ID NO: 5, amino acids 70-77 of SEQ ID NO: 5, and amino acids 116-126 of SEQ ID NO: 5.
   22. The composition of matter of any one of items 19 to 21, wherein either the first antibody and/or the second antibody binds to the epitope as bound by an antibody comprising CDRs consisting of amino acids 46-51 of SEQ ID NO: 2, amino acids 69-71 of SEQ ID NO: 2, amino acids 108-116 of SEQ ID NO: 2, amino acids 44-52 of SEQ ID NO: 3, amino acids 70-76 of SEQ ID NO: 3, amino acids 115-125 of SEQ ID NO: 3; and comprising FRs consisting of amino acids 20-45 of SEQ ID NO: 2, amino acids 52-68 of SEQ ID NO: 2, amino acids 72-107 of SEQ ID NO: 2, amino acid 117-126 of SEQ ID NO: 2, amino acids 19-43 of SEQ ID NO: 3, amino acids 53-69 of SEQ ID NO: 3, and amino acids 77-114 of SEQ ID NO: 3, amino acids 126-136 of SEQ ID NO: 3, or
comprising CDRs consisting of amino acids 47-52 of SEQ ID NO: 4, amino acids 70-72 of SEQ ID NO: 4, amino acids 109-117 of SEQ ID NO: 4, amino acids 45-52 of SEQ ID NO: 5, amino acids 70-77 of SEQ ID NO: 5, and amino acids 116-126 of SEQ ID NO: 5, and comprising FRs consisting of amino acids 21-46 of SEQ ID NO: 4, amino acids 53-69 of SEQ ID NO: 4, amino acids 73-108 of SEQ ID NO: 4, amino acids 118-127 of SEQ ID NO: 4, amino acids 20-44 of SEQ ID NO: 5, amino acids 53-69 of SEQ ID NO: 5, and amino acids 78-115 of SEQ ID NO: 5, amino acids 127-137 of SEQ ID NO: 5.
   23. The composition of matter of any one of items 19 to 22, wherein either the first or the second antibody comprises a light chain having an amino acid sequence of SEQ ID NO: 2 and a heavy chain having an amino acid sequences of SEQ ID NO: 3.
   24. The composition of matter of any one of items 19 to 23, wherein either the first or the second antibody comprises a light chain having an amino acid sequence of SEQ ID NO: 4 and a heavy chain having an amino acid sequences of SEQ ID NO: 5.
   25. The composition of matter according to any one of items 19 to 24, wherein the first or the second antibody is bound to or capable of binding to a solid phase.
   26. The composition of matter of any one of items 19 to 25, wherein the first antibody or a fragment thereof, is bound to or capable of binding to a solid phase and comprises a light chain having an amino acid sequence of SEQ ID NO: 2 and a heavy chain having an amino acid sequences of SEQ ID NO: 3, and wherein the second antibody is detectably labeled and comprises a light chain having an amino acid sequence of SEQ ID NO: 4 and a heavy chain having an amino acid sequences of SEQ ID NO: 5.
   27. The composition of matter according to any one of items 19 to 26, wherein one of the first antibody or the second antibody is biotinylated.
   28. The composition of matter according to any one of items 19 to 27, wherein one of the first antibody or the second antibody is ruthenylated.
   29. The composition of matter according to any one of items 19 to 28, wherein said composition further comprises a VEGF-A antagonist.
   30. The composition of matter according to item 29, wherein the VEGF-A antagonist is the antibody bevacizumab.
   31. A method of detecting a complex comprising human VEGF-A and a non-human or chimeric protein comprising the steps of
(a) incubating a sample comprising said complex with a detectably labeled antibody which is able to bind to or binds to human VEGF-A or the variant thereof and/or the non-human or chimeric protein, and
(b) detecting said antigen-binding protein.
   32. The method of item 31, wherein the complex comprises human VEGF-A and a non-human or chimeric antibody or antigen-binding fragment thereof, bound to VEGF-A.
   33. The method of item 31 or32, wherein the non-human or chimeric protein and the detectably labeled antibody are both capable of binding to VEGF-A, in the presence of a VEGF-A antagonist.
   34. The method of any of items 31 to 33, wherein either the non-human or chimeric protein or the detectably labeled antibody binds to the epitope as bound by an antibody comprising CDRs consisting of amino acids 46-51 of SEQ ID NO: 2, amino acids 69-71 of SEQ ID NO: 2, amino acids 108-116 of SEQ ID NO: 2, amino acids 44-52 of SEQ ID NO: 3, amino acids 70-76 of SEQ ID NO: 3, amino acids 115-125 of SEQ ID NO: 3, or
comprising CDRs consisting of amino acids 47-52 of SEQ ID NO: 4, amino acids 70-72 of SEQ ID NO: 4, amino acids 109-117 of SEQ ID NO: 4, amino acids 45-52 of SEQ ID NO: 5, amino acids 70-77 of SEQ ID NO: 5, and amino acids 116-126 of SEQ ID NO: 5.
   35. The method of any of items 31 to 34, wherein either the non-human or chimeric protein or the detectably labeled antibody comprise CDRs consisting of amino acids 46-51 of SEQ ID NO: 2, amino acids 69-71 of SEQ ID NO: 2, amino acids 108-116 of SEQ ID NO: 2, amino acids 44-52 of SEQ ID NO: 3, amino acids 70-76 of SEQ ID NO: 3, amino acids 115-125 of SEQ ID NO: 3; and comprising FRs consisting of amino acids 20-45 of SEQ ID NO: 2, amino acids 52-68 of SEQ ID NO: 2, amino acids 72-107 of SEQ ID NO: 2, amino acid 117-126 of SEQ ID NO: 2, amino acids 19-43 of SEQ ID NO: 3, amino acids 53-69 of SEQ ID NO: 3, and amino acids 77-114 of SEQ ID NO: 3, amino acids 126-136 of SEQ ID NO: 3, or
comprise CDRs consisting of amino acids 47-52 of SEQ ID NO: 4, amino acids 70-72 of SEQ ID NO: 4, amino acids 109-117 of SEQ ID NO: 4, amino acids 45-52 of SEQ ID NO: 5, amino acids 70-77 of SEQ ID NO: 5, and amino acids 116-126 of SEQ ID NO: 5, and comprising FRs consisting of amino acids 21-46 of SEQ ID NO: 4, amino acids 53-69 of SEQ ID NO: 4, amino acids 73-108 of SEQ ID NO: 4, amino acids 118-127 of SEQ ID NO: 4, amino acids 20-44 of SEQ ID NO: 5, amino acids 53-69 of SEQ ID NO: 5, and amino acids 78-115 of SEQ ID NO: 5, amino acids 127-137 of SEQ ID NO: 5.
   36. The method of any of items 31 to 35, wherein either the non-human or chimeric protein or the detectably labeled antibody comprises a light chain having an amino acid sequence of SEQ ID NO: 2 and a heavy chain having an amino acid sequences of SEQ ID NO: 3.
   37. The method of any of items 31 to 36, wherein either the non-human or chimeric protein or the detectably labeled antibody comprises a light chain having an amino acid sequence of SEQ ID NO: 4 and a heavy chain having an amino acid sequences of SEQ ID NO: 5.
   38. The method of any of items 31 to 37, wherein either the non-human or chimeric protein or the detectably labeled antibody is bound to or capable of binding to a solid phase.
   39. The method of any of items 31 to 38, wherein the non-human or chimeric protein is bound to or capable of binding to a solid phase and comprises a light chain having an amino acid sequence of SEQ ID NO: 2 and a heavy chain having an amino acid sequences of SEQ ID NO: 3, and wherein the second antibody is detectably labeled and comprises a light chain having an amino acid sequence of SEQ ID NO: 4 and a heavy chain having an amino acid sequences of SEQ ID NO: 5.
   40. The method of any of items 31 to 39, wherein the non-human or chimeric protein or the detectably labeled antibody is biotinylated or ruthenylated.
   41. The method of any of items 31 to 40, wherein prior to, simultaneously with or subsequent to step (a) the sample comprising said complex is further incubated with a VEGF-A antagonist.
   42. The method item 41, wherein the VEGF-A antagonist is the antibody bevacizumab.

### EXAMPLES

The following examples are provided to illustrate, but not to limit the presently claimed invention.

### Example 1: Biacore Epitope Binning

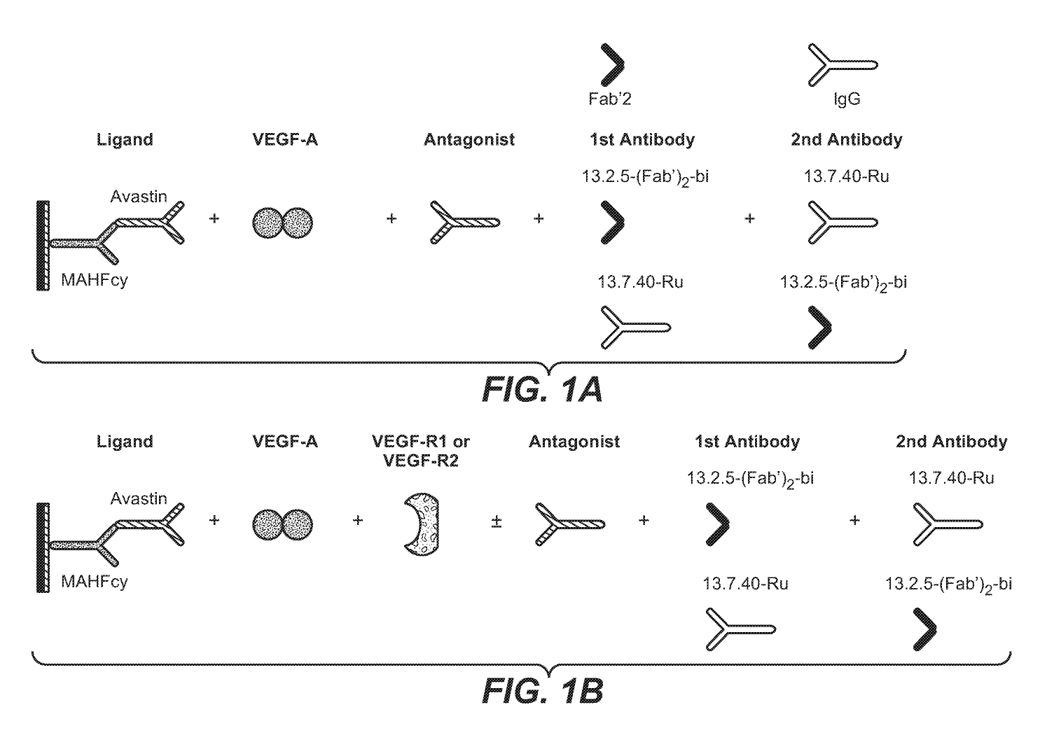

A Biacore T200 instrument (GE Healthcare) was used for a ternary epitope binning experiment in order to assess the epitope accessibility of 3 monoclonal antibodies or antibody fragment conjugates on dimeric VEGF-A 121 (see FIGS. 1A-1B). Antibodies with affinity for VEGF-A described here are rH-4.6.1-IgG (AVASTIN®), 13.2.5-F(ab′)2-Bi, and 13.7.40-Ru. A SC1 sensor was mounted into the Biacore system and was normalized in HBSN buffer (10 mM HEPES pH 7.4, 150 mM NaCl) according to the manufacturer's instructions. The system buffer was changed to PBS buffer pH 7.4 with 5% DMSO and 0.05% Tween20. The sample buffer was the system buffer supplemented with 1 mg/ml CMD (Carboxymethyldextran, Sigma #86524). The system operated at 25° C. An antibody capture system was immobilized on the mounted SC1 sensor. 1600 RU of a monoclonal murine anti-human FC gamma-pan capture antibody (MAHFcg-pan, Roche) was immobilized on all sensor flow cells by conventional EDC/NHS chemistry as described by the supplier. The capture system was regenerated by a 15 sec injection at 20 μl/min with 10 mM NaOH followed by two injections for 1 min each at 20 μl/min with 10 mM glycin buffer pH 2.5.

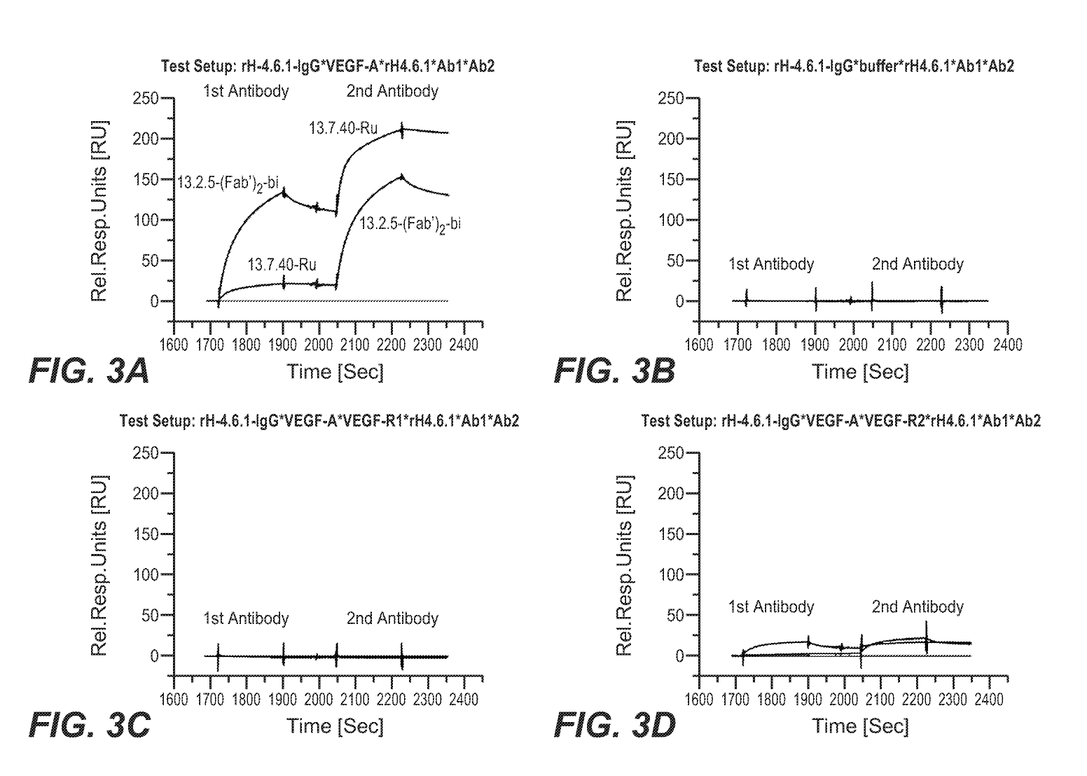

On flow cell 1 and flow cell 2, 75 nM of the primary antibody rH-4.6.1-IgG were injected at 30 μl/min for 1 min at 5 μl/min. The capture system on both flow cells was saturated by a 3 min injection at 30 μl/min using a mixture of 2 μM human normal IgG (Roche) with 1 μM M-33 (Roche) in sample buffer. At this stage of preparation the assay setup on flow cell 1 served as an antigen-free reference. 150 nM VEGF-A (VEGF-A 121, Peprotech) were injected into flow cell 2 at 30 μl/min for 3 min to be displayed on the sensor by rH-4.6.1-IgG. In order to saturate potentially accessible second antibody binding sites on the displayed dimeric VEGFs 121, 75 nM rH-4.6.1-IgG were again injected into flow cells 1 and 2 at 20 μl/min for 3 min association time. 75 nM of the biotinylated antibody fragment 13.2.5-F(ab′)2-Bi were injected into flow cells 1 and 2 at 20 μl/min for 3 min followed by a consecutive injection of 75 nM 13.7.40-Ru for 3 min at 30 μl/min. The black line in image (FIG. 3A) shows the consecutive injections into flow cell 2 of first, the 13.2.5-F(ab′)2-Bi conjugate and second, the 13.7.40-Ru conjugate after the sensor surface has been prepared as described above. The dotted black line shows the signal levels of the two consecutive injections of 13.2.5-F(ab′)2-Bi and 13.7.40-Ru without the presence of VEGF-A 121 on flow cell 1 as a reference. On flow cell 2, 13.7.40-Ru shows an increasing binding signal, which is higher than the expected signal saturation level of the preceding 13.2.5-F(ab′)2-Bi injection. This indicates VEGF-A 121 epitope accessibility for both conjugates. On flow cell 1, where the VEGF-A antigen has been omitted, no interaction was detectable. Control experiments wherein either no VEGF-A (FIG. 3B) was present, or wherein also VEGFA-R1 (FIG. 3C) or VEGFA-R2 (FIG. 3D) was present evidenced the binding of the tested antibodies was VEGF-A specific and that the antibodies bind to epitopes of VEGF-A to which the VEGF-A receptors bind, or which are covered by the binding of the VEGF-A receptors.

### Example 2: Elecsys Competition Assay

Measurements were carried out in a sandwich assay format. Signal detection in the cobas® e601 analyzer is based on electrochemiluminescense. In this sandwich assay the biotin-conjugate (i.e. the capture antibody) is immobilized on the surface of a streptavidin-coated magnetic bead. The detection-antibody bears a complexed ruthenium cation as the signaling moiety. In the presence of analyte, the chromogenic ruthenium complex is bridged to the solid phase and emits light at 620 nm after excitation at the platinum electrode comprised in the measuring cell of the cobas® e601 analyzer. The signal output is in arbitrary light units.

The experimental VEGF-A antigen assay was conducted as follows. An antigen positive sample was spiked with an at least 50-fold molar excess of a VEGF-A antagonist and incubated for 30 min to allow equilibrium binding to VEGF-A. 50 μl sample preincubated with a VEGF-A antagonist were measured on a cobas® e601 analyzer with 50 μl 1 μg/ml capture antibody-biotin conjugate and 50 μl of 1 μg/ml detection antibody ruthenium label conjugate in physiological buffer at pH 7.0 and comprising 100 mM potassium phosphate and 225 mM KCl. After 9 minutes incubation time 50 μl streptavidin-coated paramagnetic microparticles were added and incubated for further 9 minutes. Afterwards, the VEGF-A antigen was detected (via the electrochemiluminescent signal generated in these experiments). VEGF-A was detected using Elecsys competition assay in the presence of AVASTIN®.

1. A method for measuring the level of VEGF-A, in the presence of a VEGF-A antagonist, the method comprising:
incubating a sample with a first and a second antibody,
wherein said first and said second antibody, are capable of binding to VEGF-A in the presence of the VEGF-A antagonist, and wherein the binding of said first and of said second antibody does not interfere with each other,
wherein one of said antibodies is detectably labeled, thereby forming a detectably labeled complex comprising the first antibody, VEGF-A, and the second antibody, and
detecting the complex formed, thereby measuring the level of VEGF-A in the presence of a VEGF-A antagonist.

2. The method of claim 1, wherein the VEGF-A antagonist is a VEGF-A-binding polypeptide.

3. The method of claim 1, wherein the VEGF-A antagonist is bevacizumab.

4. The method of claim 1, wherein the first and/or the second antibody binds to an epitope which are covered by or bound by a VEGF receptor.

5. The method of claim 1, wherein the first or the second antibody comprises CDRs consisting of amino acids 46-51 of SEQ ID NO: 2, amino acids 69-71 of SEQ ID NO: 2, amino acids 108-116 of SEQ ID NO: 2, amino acids 44-52 of SEQ ID NO: 3, amino acids 70-76 of SEQ ID NO: 3, amino acids 115-125 of SEQ ID NO: 3, or
comprises CDRs consisting of amino acids 47-52 of SEQ ID NO: 4, amino acids 70-72 of SEQ ID NO: 4, amino acids 109-117 of SEQ ID NO: 4, amino acids 45-52 of SEQ ID NO: 5, amino acids 70-77 of SEQ ID NO: 5, and amino acids 116-126 of SEQ ID NO: 5.

6. The method of claim 1, wherein the first or the second antibody comprises a light chain having an amino acid sequences of SEQ ID NO: 2 and a heavy chain having an amino acid sequences of SEQ ID NO:3.

7. The method of claim 6, wherein the first or the second antibody comprises a light chain having an amino acid sequences of SEQ ID NO: 4 and a heavy chain having an amino acid sequences of SEQ ID NO: 5.

8. The method of claim 1, wherein the first or the second antibody is bound to or capable of binding to a solid phase.

9. The method of claim 1, wherein the first antibody or the second antibody is biotinylated and wherein the other antibody is ruthenylated.

10. The method of claim 1, wherein the sample is derived from a patient treated with a VEGF-A antagonist.

11. The method of claim 10, wherein the sample is body fluid.

12. A kit for measuring the level of VEGF-A or a variant thereof, in the presence of a VEGF-A antagonist, the kit comprising: a first and a second antibody,
wherein both said first and said second antibody are capable of binding to VEGF-A in the presence of the VEGF-A antagonist and wherein the binding of said first and of said second antibody does not interfere with each other, and
wherein one of said antibodies is detectably labeled.

13. The kit according to claim 12, wherein said kit comprises a first and a second antibody capable of binding to VEGF-A in the presence of the VEGF-A antagonist, and wherein the binding of said first and of said second antibody does not interfere with each other, and wherein one of the antibodies is detectably labeled, thereby forming a detectably labeled complex comprising the first antibody, VEGF-A, and the second antibody and wherein; antibodies as specified in any one of claims 4 to 9
a) the first and/or the second antibody binds to an epitope which is covered by or bound by a VEGF receptor;
b) the first or the second antibody comprises CDRs consisting of amino acids 46-51 of SEQ ID NO: 2, amino acids 69-71 of SEQ ID NO: 2, amino acids 108-116 of SEQ ID NO: 2, amino acids 44-52 of SEQ ID NO: 3, amino acids 70-76 of SEQ ID NO: 3, amino acids 115-125 of SEQ ID NO: 3, or
comprises CDRs consisting of amino acids 47-52 of SEQ ID NO: 4, amino acids 70-72 of SEQ ID NO: 4, amino acids 109-117 of SEQ ID NO: 4, amino acids 45-52 of SEQ ID NO: 5, amino acids 70-77 of SEQ ID NO: 5, and amino acids 116-126 of SEQ ID NO: 5;
c) the first or the second antibody comprises a light chain having an amino acid sequences of SEQ ID NO: 2 and a heavy chain having an amino acid sequences of SEQ ID NO:3:
d) the first or the second antibody comprises a light chain having an amino acid sequences of SEQ ID NO: 4 and a heavy chain having an amino acid sequences of SEQ ID NO: 5;
e) the first antibody and the second antibody of a), b), c), or d), further wherein the first antibody or the second antibody is biotinylated and wherein the other antibody is ruthenylated; or
f) the first antibody and the second antibody of a), b), c), or d), further wherein the first or the second antibody is bound to or capable of binding to a solid phase.

14. A composition of matter comprising a first and a second antibody,
wherein said first and said second antibody are both capable of binding to VEGF-A in the presence of a VEGF-A antagonist,
wherein the binding of said first and of said second antibody does not interfere with each other, and
wherein one of said antibodies is detectably labeled.

15. A method of detecting a complex comprising human VEGF-A and a non-human or chimeric protein comprising the steps of
(a) incubating a sample comprising said complex with a detectably labeled antibody or antigen-binding fragment thereof, which is able to bind to or binds to human VEGF-A and/or the non-human or chimeric protein, and
(b) detecting said antigen-binding protein.
